SEQUENCE LISTING (177 sequences)

SEQ ID NO: 1 - DNA, 1507 bp, Influenza A virus
  strain A/Viet Nam/1203/2004, serotype H5N1 nucleocapsid protein (NP) gene
atggcgtctcaaggcaccaaacgatcttatgaacagatggaaactggtggggaacgccagaatgctactgagatcagggcatctgttggaagaatggttagtggcattgggaggttctacatacagatgtgcacagaactcaaactcagtgactatgaagggaggctgatccagaacagcataacaatagagagaatggtactctctgcatttgatgaaagaaggaacagatacctggaagaacaccccagtgcgggaaaggacccgaagaagactggaggtccaatttatcggaggagagacgggaaatgggtgagagagctaattctgtacgacaaagaggagatcaggaggatttggcgtcaagcgaacaatggagaggacgcaactgctggtcttacccacctgatgatatggcattccaatctaaatgatgccacatatcagagaacgagagctctcgtgcgtactggaatggacccaaggatgtgctctctgatgcaagggtcaactctcccgaggagatctggagctgccggtgcagcagtaaagggggtagggacaatggtgatggagctgattcggatgataaaacgagggatcaacgaccggaatttctggagaggcgaaaatggaagaagaacaaggattgcatatgagagaatgtgcaacatcctcaaagggaaattccaaacagcagcacaaagagcaatgatggatcaagtgcgagagagcagaaatcctgggaatgctgaaattgaagatctcatttttctggcacggtctgcactcatcctgagaggatcagtggcccataagtcctgcttgcctgcttgtgtgtacggacttgcagtggccagtggatatgactttgagagagaagggtactctctggttggaatagatcctttccgcctgcttcaaaacagccaggtctttagtctcattagaccaaatgagaatccagcacataagagtcaattagtgtggatggcatgccactctgcagcatttgaggaccttagagtctcaagtttcatcagagggacaagagtggtcccaagaggacagctatccaccagaggggttcaaattgcttcaaatgagaacatggaggcaatggactccaacactcttgaactgagaagcagatattgggctataagaaccagaagcggaggaaacaccaaccagcagagggcatctgcaggacagatcagcgttcagcccactttctcggtccagagaaaccttcccttcgaaagagcgaccattatggcagcatttacaggaaatactgagggcagaacgtctgacatgaggactgaaatcataagaatgatggaaagtgccagaccagaagatgtgtcattccaggggcggggagtcttcgagctctcggacgaaaaggcaacgaacccgatcgtgccttcctttgacatgaataatgaaggatcttatttcttcggagacaatgcagaggagtatgacaattaaagaaaaatac

SEQ ID NO: 2 - DNA, 1565 bp, Influenza A virus
  strain A/Puerto Rico/8/34/Mount Sinai, serotype H1N1 nucleocapsid protein (NP) gene
agcaaaagcagggtagataatcactcactgagtgacatcaaaatcatggcgtcccaaggcaccaaacggtcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaatacctggaagaacatcccagtgcggggaaagatcctaagaaaactggaggacctatatacaggagagtaaacggaaagtggatgagagaactcatcctttatgacaaagaagaaataaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcaggatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagagagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagagggaagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 3 - DNA, 1565 bp, Influenza A virus
  strain A/Goose/Guangdong/1/96, serotype H5N1 nucleocapsid protein (NP) gene
agcaaaagcagggtagataatcactcactgagtgacatcaacatcatggcgtctcagggcaccaaacgatcttatgaacagatggaaactggtggagaacgccagaatgctactgagatcagagcatctgttggaagaatggttggtggaattgggaggttttatatacagatgtgcactgaactcaaactcagcgactatgaaggaaggctgattcagaacagcataacaatagagagaatggttctctctgcatttgatgaaaggaggaacaaatacctggaagaacatcccagtgcggggaaggacccaaagaaaactggaggtccaatctaccgaagaagagacggaaaatgggtgagagagctgattctgtatgacaaagaggagatcaggagaatttggcgtcaagcgaacaatggagaagatgcaactgctggtctcactcacatgatgatctggcattccaatctaaatgatgccacataccagagaacaagagctctcgtgcgtactgggatggaccctagaatgtgctctctgatgcaaggatcaactctcccgaggagatctggagctgctggtgcggcagtaaagggagtcggaacgatggtgatggaactaattcggatgataaagcgagggattaacgatcggaatttctggagaggtgaaaatgggcgaagaacaagaattgcatatgagagaatgtgcaacatcctcaaagggaaattccaaacagcagcacaaagagcaatgatggatcaggtacgggaaagcagaaatcctgggaatgctgagattgaagatctcatatttctggcacggtctgcactcatcctgagaggatcagtggcccacaagtcctgcttgcctgcttgtgtgtacgggcttgccgtggccagtggatatgactttgagagagaagggtactctctggtcgggattgatcctttccgtctgctgcaaaacagccaggtctttagtctaattagaccaaatgagaatccagcacataaaagtcaattggtgtggatggcatgccattctgcagcatttgaagatctgagagtctcaagcttcatcagagggacaagagtggccccaaggggacaactatctactagaggagttcaaattgcttcaaatgagaacatggaaacaatggactccagcactcttgaactgagaagcagatattgggctataaggaccaggagtggaggaaacaccaaccagcagagagcatctgcaggacaaatcagtgtgcagcctactttctcggtacagagaaatcttcccttcgaaagagcgaccattatggcggcattcacagggaatacagagggcagaacatctgacatgaggactgaaatcataaggatgatggaaagctccagaccagaagatgtgtctttccaggggcggggagtcttcgagctctcggacgaaaaggcaacgaacccgatcgtgccttcctttgacatgagtaatgaaggatcttatttcttcggagacaatgcagaggaatatgacaattgaagaaaaatacccttgtttctact

SEQ ID NO: 4 - DNA, 2151 bp, Influenza A virus
  strain A/Viet Nam/1203/2004, serotype H5N1 RNA polymerase (PA) gene
atggaagactttgtgcgacaatgcttcaatccaatgattgtcgagcttgcggaaaaggcaatgaaagaatatggggaagatccgaaaatcgaaacgaacaagtttgctgcaatatgcacacacttggaggtctgtttcatgtattcggattttcactttattgatgaacggagtgaatcaataattgtagaatctggagatccgaatgcattattgaaacaccgatttgaaataattgaaggaagagaccgaacgatggcctggactgtggtgaatagtatctgcaacaccacaggagttgagaaacctaaatttctcccagatttgtatgactacaaagagaaccgattcatcgaaattggagtgacacggagggaagttcatacatactatctggagaaagccaacaagataaaatccgaggagacacatattcacatattctcattcacaggggaggaaatggccaccaaagcggactacacccttgatgaagagagcagggcaagaattaaaaccaggctgttcaccataaggcaggaaatggccagtaggggtctatgggattcctttcgtcaatccgagagaggcgaagagacaattgaagaaaaatttgaaatcactggaaccatgcgcagacttgcagaccaaagtctcccaccgaacttctccagccttgaaaactttagagcctatgtggatggattcgaaccgaacggctgcattgagggcaagctttctcaaatgtcaaaagaagtgaatgctagaattgagccatttttgaagacaacgccacgccctctcagactacctgatgggcctccttgctctcagcggtcgaagttcttgctgatggatgcccttaaattaagcatcgaagacccgagtcatgagggggaggggataccactatacgatgcaatcaaatgcatgaagacatttttcggctggaaagagcccaacatcgtgaaaccacatgaaaaaggtataaaccccaattacctcctggcttggaagcaagtgctggcagaactccaagatattgaaaatgaggagaaaatcccaaaaacaaagaacatgaaaaaaacaagccagttgaagtgggcactcggtgagaacatggcaccagagaaagtagactttgaggactgcaaagatgttagcgatctaagacagtatgacagtgatgaaccagagtctagatcactagcaagctggattcagagtgaattcaacaaggcatgtgaattgacagattcgatttggattgaactcgatgaaataggagaagacgtagctccaattgagcacattgcaagtatgagaaggaactattttacagcggaagtatcccattgcagggccactgaatacataatgaagggagtgtacataaacacagccctgttgaatgcatcctgtgcagccatggatgactttcaactgattccaatgataagcaaatgcagaaccaaagaaggaagacggaaaactaatctgtatggattcattataaaagggagatcccacttgaggaatgataccgatgtggtaaattttgtgagtatggaattctctcttactgatccgaggctggagccacacaagtgggaaaagtactgtgtcctcgagataggagacatgctcctccggactgcagtaggccaagtttcgaggcccatgttcctgtatgtaagaaccaatggaacctccaagatcaaaatgaaatggggcatggaaatgaggcgatgccttcttcaatcccttcaacaaattgaaagcatgattgaagccgagtcttctgtcaaagagaaggacatgaccaaagaattctttgaaaacaaatcagaaacatggccgattggagagtcccccaagggagtggaggaaggctccatcggaaaggtgtgcagaaccttgctggcgaagtctgtgttcaacagtttatatgcatctccacaactcgaggggttttcagctgaatcaagaaaattgcttctcattgctcaggcacttagggacaacctggaacctgggaccttcgatcttggagggctatatgaagcaattgaggagtgcctgattaacgatccctgggttttgcttaatgcgtcttggttcaactccttcctcgcacatgcactgaaatag

SEQ ID NO: 5 - DNA, 2233 bp, Influenza A virus
  strain A/Puerto Rico/8/34/Mount Sinai, serotype H1N1 RNA polymerase (PA) gene
agcgaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgctagcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 6 - RNA, 19 nt, Artificial Sequence
  Description of Artificial SequenceInfluenza A virus nucleocapsid protein (NP) target siRNA sense strand
ggacgcaacugcuggucuu

SEQ ID NO: 7 - RNA, 19 nt, Artificial Sequence
  Description of Artificial SequenceInfluenza A virusnucleocapsid protein (NP) target siRNA antisense strand
aagaccagcaguugcgucc

SEQ ID NO: 8 - RNA, 19 nt, Artificial Sequence
  Description of Artificial SequenceInfluenza A virusnucleocapsid protein (NP) target siRNA sense strand
gcauuccaaucuaaaugau

SEQ ID NO: 9 - RNA, 19 nt, Artificial Sequence
  Description of Artificial SequenceInfluenza A virusnucleocapsid protein (NP) target siRNA antisense strand
aucauuuagauuggaaugc

SEQ ID NO: 10 - RNA, 19 nt, Artificial Sequence
  Description of Artificial SequenceInfluenza A virusnucleocapsid protein (NP) target siRNA sense strand
cgaccggaauuucuggaga

SEQ ID NO: 11 - RNA, 19 nt, Artificial Sequence
  Description of Artificial SequenceInfluenza A virusnucleocapsid protein (NP) target siRNA antisense strand
ucuccagaaauuccggucg

SEQ ID NO: 12 - RNA, 19 nt, Artificial Sequence
  Description of Artificial SequenceInfluenza A virusnucleocapsid protein (NP) target siRNA sense strand
gaacaaggauugcauauga

SEQ ID NO: 13 - RNA, 19 nt, Artificial Sequence
  Description of Artificial SequenceInfluenza A virusnucleocapsid protein (NP) target siRNA antisense strand
ucauaugcaauccuuguuc

SEQ ID NO: 14 - RNA, 19 nt, Artificial Sequence
  Description of Artificial SequenceInfluenza A virusnucleocapsid protein (NP) target siRNA sense strand
acagccaggucuuuagucu

SEQ ID NO: 15 - RNA, 19 nt, Artificial Sequence
  Description of Artificial SequenceInfluenza A virusnucleocapsid protein (NP) target siRNA antisense strand
agacuaaagaccuggcugu

SEQ ID NO: 16 - RNA, 19 nt, Artificial Sequence
  Description of Artificial SequenceInfluenza A virusnucleocapsid protein (NP) target siRNA sense strand
ugaggaccuuagagucuca

SEQ ID NO: 17 - RNA, 19 nt, Artificial Sequence
  Description of Artificial SequenceInfluenza A virusnucleocapsid protein (NP) target siRNA antisense strand
ugagacucuaagguccuca

SEQ ID NO: 18 - RNA, 19 nt, Artificial Sequence
  Description of Artificial SequenceInfluenza A virusnucleocapsid protein (NP) target siRNA sense strand
agagaaaccuucccuucga

SEQ ID NO: 19 - RNA, 19 nt, Artificial Sequence
  Description of Artificial SequenceInfluenza A virusnucleocapsid protein (NP) target siRNA antisense strand
ucgaagggaagguuucucu

SEQ ID NO: 20 - RNA, 19 nt, Artificial Sequence
  Description of Artificial SequenceInfluenza A virusnucleocapsid protein (NP) target siRNA sense strand
cgaccauuauggcagcauu

SEQ ID NO: 21 - RNA, 19 nt, Artificial Sequence
  Description of Artificial SequenceInfluenza A virusnucleocapsid protein (NP) target siRNA antisense strand
aaugcugccauaauggucg

SEQ ID NO: 22 - RNA, 19 nt, Artificial Sequence
  Description of Artificial SequenceInfluenza A virusnucleocapsid protein (NP) target siRNA sense strand
ggacugaaaucauaagaau

SEQ ID NO: 23 - RNA, 19 nt, Artificial Sequence
  Description of Artificial SequenceInfluenza A virusnucleocapsid protein (NP) target siRNA antisense strand
auucuuaugauuucagucc

SEQ ID NO: 24 - RNA, 19 nt, Artificial Sequence
  Description of Artificial SequenceInfluenza A virusnucleocapsid protein (NP) target siRNA sense strand
ugacaugaauaaugaagga

SEQ ID NO: 25 - RNA, 19 nt, Artificial Sequence
  Description of Artificial SequenceInfluenza A virusnucleocapsid protein (NP) target siRNA antisense strand
uccuucauuauucauguca

SEQ ID NO: 26 - RNA, 19 nt, Artificial Sequence
  Description of Artificial SequenceInfluenza A virus RNApolymerase (PA) target siRNA sense strand
cgaacaaguuugcugcaau

SEQ ID NO: 27 - RNA, 19 nt, Artificial Sequence
  Description of Artificial SequenceInfluenza A virus RNApolymerase (PA) target siRNA antisense strand
auugcagcaaacuuguucg

SEQ ID NO: 28 - RNA, 19 nt, Artificial Sequence
  Description of Artificial SequenceInfluenza A virus RNApolymerase (PA) target siRNA sense strand
ugaacggagugaaucaaua

SEQ ID NO: 29 - RNA, 19 nt, Artificial Sequence
  Description of Artificial SequenceInfluenza A virus RNApolymerase (PA) target siRNA antisense strand
uauugauucacuccguuca

SEQ ID NO: 30 - RNA, 19 nt, Artificial Sequence
  Description of Artificial SequenceInfluenza A virus RNApolymerase (PA) target siRNA sense strand
cgaaugcauuauugaaaca

SEQ ID NO: 31 - RNA, 19 nt, Artificial Sequence
  Description of Artificial SequenceInfluenza A virus RNApolymerase (PA) target siRNA antisense strand
uguuucaauaaugcauucg

SEQ ID NO: 32 - RNA, 19 nt, Artificial Sequence
  Description of Artificial SequenceInfluenza A virus RNApolymerase (PA) target siRNA sense strand
accuaaauuucucccagau

SEQ ID NO: 33 - RNA, 19 nt, Artificial Sequence
  Description of Artificial SequenceInfluenza A virus RNApolymerase (PA) target siRNA antisense strand
aucugggagaaauuuaggu

SEQ ID NO: 34 - RNA, 19 nt, Artificial Sequence
  Description of Artificial SequenceInfluenza A virus RNApolymerase (PA) target siRNA sense strand
cuaaauuucucccagauuu

SEQ ID NO: 35 - RNA, 19 nt, Artificial Sequence
  Description of Artificial SequenceInfluenza A virus RNApolymerase (PA) target siRNA antisense strand
aaaucugggagaaauuuag

SEQ ID NO: 36 - RNA, 19 nt, Artificial Sequence
  Description of Artificial SequenceInfluenza A virus RNApolymerase (PA) target siRNA sense strand
gagaaccgauucaucgaaa

SEQ ID NO: 37 - RNA, 19 nt, Artificial Sequence
  Description of Artificial SequenceInfluenza A virus RNApolymerase (PA) target siRNA antisense strand
uuucgaugaaucgguucuc

SEQ ID NO: 38 - RNA, 19 nt, Artificial Sequence
  Description of Artificial SequenceInfluenza A virus RNApolymerase (PA) target siRNA sense strand
agaaccgauucaucgaaau

SEQ ID NO: 39 - RNA, 19 nt, Artificial Sequence
  Description of Artificial SequenceInfluenza A virus RNApolymerase (PA) target siRNA antisense strand
auuucgaugaaucgguucu

SEQ ID NO: 40 - RNA, 19 nt, Artificial Sequence
  Description of Artificial SequenceInfluenza A virus RNApolymerase (PA) target siRNA sense strand
ggagggaaguucauacaua

SEQ ID NO: 41 - RNA, 19 nt, Artificial Sequence
  Description of Artificial SequenceInfluenza A virus RNApolymerase (PA) target siRNA antisense strand
uauguaugaacuucccucc

SEQ ID NO: 42 - RNA, 19 nt, Artificial Sequence
  Description of Artificial SequenceInfluenza A virus RNApolymerase (PA) target siRNA sense strand
agaagugaaugcuagaauu

SEQ ID NO: 43 - RNA, 19 nt, Artificial Sequence
  Description of Artificial SequenceInfluenza A virus RNApolymerase (PA) target siRNA antisense strand
aauucuagcauucacuucu

SEQ ID NO: 44 - RNA, 19 nt, Artificial Sequence
  Description of Artificial SequenceInfluenza A virus RNApolymerase (PA) target siRNA sense strand
gcaaucaaaugcaugaaga

SEQ ID NO: 45 - RNA, 19 nt, Artificial Sequence
  Description of Artificial SequenceInfluenza A virus RNApolymerase (PA) target siRNA antisense strand
ucuucaugcauuugauugc

SEQ ID NO: 46 - RNA, 19 nt, Artificial Sequence
  Description of Artificial SequenceInfluenza A virus RNApolymerase (PA) target siRNA sense strand
ucaaaugcaugaagacauu

SEQ ID NO: 47 - RNA, 19 nt, Artificial Sequence
  Description of Artificial SequenceInfluenza A virus RNApolymerase (PA) target siRNA antisense strand
aaugucuucaugcauuuga

SEQ ID NO: 48 - RNA, 19 nt, Artificial Sequence
  Description of Artificial SequenceInfluenza A virus RNApolymerase (PA) target siRNA sense strand
ggaugacuuucaacugauu

SEQ ID NO: 49 - RNA, 19 nt, Artificial Sequence
  Description of Artificial SequenceInfluenza A virus RNApolymerase (PA) target siRNA antisense strand
aaucaguugaaagucaucc

SEQ ID NO: 50 - RNA, 19 nt, Artificial Sequence
  Description of Artificial SequenceInfluenza A virus RNApolymerase (PA) target siRNA sense strand
ucaacugauuccaaugaua

SEQ ID NO: 51 - RNA, 19 nt, Artificial Sequence
  Description of Artificial SequenceInfluenza A virus RNApolymerase (PA) target siRNA antisense strand
uaucauuggaaucaguuga

SEQ ID NO: 52 - RNA, 19 nt, Artificial Sequence
  Description of Artificial SequenceInfluenza A virus RNApolymerase (PA) target siRNA sense strand
ggaauucucucuuacugau

SEQ ID NO: 53 - RNA, 19 nt, Artificial Sequence
  Description of Artificial SequenceInfluenza A virus RNApolymerase (PA) target siRNA antisense strand
aucaguaagagagaauucc

SEQ ID NO: 54 - DNA, 21 bp, Artificial Sequence
  Description of Combined DNA/RNA Molecule Influenza A virusnucleocapsid protein (NP) target siRNA NP 180 sense strand
  Description of Artificial SequenceInfluenza A virus nucleocapsid protein (NP) target siRNA NP 180 sense strand
cgaacucaaacucagugautt

SEQ ID NO: 55 - DNA, 21 bp, Artificial Sequence
  Description of Combined DNA/RNA Molecule Influenza A virusnucleocapsid protein (NP) target siRNA NP 180 antisensestrand
  Description of Artificial SequenceInfluenza A virusnucleocapsid protein (NP) target siRNA NP 180 antisense strand
aucacugaguuugaguucgtt

SEQ ID NO: 56 - DNA, 21 bp, Artificial Sequence
  Description of Combined DNA/RNA Molecule Influenza A virusnucleocapsid protein (NP) target siRNA NP 952 sense strand
  Description of Artificial SequenceInfluenza A virusnucleocapsid protein (NP) target siRNA NP 952 sense strand
ccuuucagacugcuucaaatt

SEQ ID NO: 57 - DNA, 21 bp, Artificial Sequence
  Description of Combined DNA/RNA Molecule Influenza A virusnucleocapsid protein (NP) target siRNA NP 952 antisensestrand
  Description of Artificial SequenceInfluenza A virus nucleocapsid protein (NP) target siRNA NP 952 antisense strand
uuugaagcagucugaaaggtt

SEQ ID NO: 58 - DNA, 21 bp, Artificial Sequence
  Description of Combined DNA/RNA Molecule Influenza A virusnucleocapsid protein (NP) target siRNA NP 411 sense strand
  Description of Artificial Sequence Influenza A virusnucleocapsid protein (NP) target siRNA NP 411 sense strand
agcuaauaauggugacgautt

SEQ ID NO: 59 - DNA, 21 bp, Artificial Sequence
  Description of Combined DNA/RNA Molecule Influenza A virusnucleocapsid protein (NP) target siRNA NP 411 antisensestrand
  Description of Artificial SequenceInfluenza A virus nucleocapsid protein (NP) target siRNA NP 411 antisense strand
aucgucaccauuauuagcutt

SEQ ID NO: 60 - DNA, 21 bp, Artificial Sequence
  Description of Combined DNA/RNA Molecule Influenza A virus nucleocapsid protein (NP) target siRNA NP 604 sense strand
  Description of Artificial SequenceInfluenza A virusnucleocapsid protein (NP) target siRNA NP 604 sense strand
ggaacaauggugauggaautt

SEQ ID NO: 61 - DNA, 21 bp, Artificial Sequence
  Description of Combined DNA/RNA Molecule Influenza A virusnucleocapsid protein (NP) target siRNA NP 604 antisensestrand
  Description of Artificial SequenceInfluenza A virusnucleocapsid protein (NP) target siRNA NP 604 antisense strand
auuccaucaccauuguucctt

SEQ ID NO: 62 - DNA, 21 bp, Artificial Sequence
  Description of Combined DNA/RNA Molecule Influenza A virusnucleocapsid protein (NP) target siRNA NP 929 sense strand
  Description of Artificial SequenceInfluenza A virusnucleocapsid protein (NP) target siRNA NP 929 sense strand
gauacucucuagucggaautt

SEQ ID NO: 63 - DNA, 21 bp, Artificial Sequence
  Description of Combined DNA/RNA Molecule Influenza A virusnucleocapsid protein (NP) target siRNA NP 929 antisensestrand
  Description of Artificial SequenceInfluenza A virusnucleocapsid protein (NP) target siRNA NP 929 antisense strand
auuccgacuagagaguauctt

SEQ ID NO: 64 - DNA, 21 bp, Artificial Sequence
  Description of Combined DNA/RNA Molecule Influenza A virusnucleocapsid protein (NP) target siRNA NP 1116 sense strand
  Description of Artificial SequenceInfluenza A virusnucleocapsid protein (NP) target siRNA NP 1116 sense strand
gcuuuccacuagaggaguutt

SEQ ID NO: 65 - DNA, 21 bp, Artificial Sequence
  Description of Combined DNA/RNA Molecule Influenza A virusnucleocapsid protein (NP) target siRNA NP 1116 antisensestrand
  Description of Artificial SequenceInfluenza A virusnucleocapsid protein (NP) target siRNA NP 1116 antisense strand
aacuccucuaguggaaagctt

SEQ ID NO: 66 - DNA, 21 bp, Artificial Sequence
  Description of Combined DNA/RNA Molecule Influenza A virusnucleocapsid protein (NP) target siRNA NP 1496 sense strand
  Description of Artificial SequenceInfluenza A virusnucleocapsid protein (NP) target siRNA NP 1496 sense strand
ggaucuuauuucuucggagtt

SEQ ID NO: 67 - DNA, 21 bp, Artificial Sequence
  Description of Combined DNA/RNA Molecule Influenza A virusnucleocapsid protein (NP) target siRNA NP 1496 antisensestrand
  Description of Artificial SequenceInfluenza A virus nucleocapsid protein (NP) target siRNA NP 1496 antisense strand
cuccgaagaaauaagaucctt

SEQ ID NO: 68 - DNA, 21 bp, Artificial Sequence
  Description of Combined DNA/RNA Molecule Influenza A virusRNA polymerase (PA) target siRNA PA 626 sense strand
  Description of Artificial SequenceInfluenza A virus RNApolymerase (PA) target siRNA PA 626 sense strand
cacagagaacaauagguaatt

SEQ ID NO: 69 - DNA, 21 bp, Artificial Sequence
  Description of Combined DNA/RNA Molecule Influenza A virusRNA polymerase (PA) target siRNA PA 626 antisense strand
  Description of Artificial SequenceInfluenza A virus RNApolymerase (PA) target siRNA PA 626 antisense strand
uuaccuauuguucucugugtt

SEQ ID NO: 70 - DNA, 21 bp, Artificial Sequence
  Description of Combined DNA/RNA Molecule Influenza A virusRNA polymerase (PA) target siRNA PA 848 sense strand
  Description of Artificial SequenceInfluenza A virus RNA polymerase (PA) target siRNA PA 848 sense strand
gcaaugagaagaaagcaaatt

SEQ ID NO: 71 - DNA, 21 bp, Artificial Sequence
  Description of Combined DNA/RNA Molecule Influenza A virusRNA polymerase (PA) target siRNA PA848 antisense strand
  Description of Artificial SequenceInfluenza A virus RNA polymerase (PA) target siRNA PA848 antisense strand
uuugcuuucuucucauugctt

SEQ ID NO: 72 - DNA, 21 bp, Artificial Sequence
  Description of Combined DNA/RNA Molecule Influenza A virusRNA polymerase (PA) target siRNA PA 1467 sense strand
  Description of Artificial SequenceInfluenza A virus RNA polymerase (PA) target siRNA PA 1467 sense strand
gucuuacauaaacagaacatt

SEQ ID NO: 73 - DNA, 21 bp, Artificial Sequence
  Description of Combined DNA/RNA Molecule Influenza A virusRNA polymerase (PA) target siRNA PA 1467 antisense strand
  Description of Artificial SequenceInfluenza A virus RNApolymerase (PA) target siRNA PA 1467 antisense strand
uguucuguuuauguaagactt

SEQ ID NO: 74 - DNA, 21 bp, Artificial Sequence
  Description of Combined DNA/RNA Molecule Influenza A virusRNA polymerase (PA) target siRNA PA 1898 sense strand
  Description of Artificial SequenceInfluenza A virus RNA polymerase (PA) target siRNA PA 1898 sense strand
gcaacccacugaacccauutt

SEQ ID NO: 75 - DNA, 21 bp, Artificial Sequence
  Description of Combined DNA/RNA Molecule Influenza A virusRNA polymerase (PA) target siRNA PA 1898 antisense strand
  Description of Artificial SequenceInfluenza A virus RNA polymerase (PA) target siRNA PA 1898 antisense strand
aauggguucaguggguugctt

SEQ ID NO: 76 - DNA, 21 bp, Artificial Sequence
  Description of Combined DNA/RNA Molecule Influenza A virusRNA polymerase (PA) target siRNA PA 2256 sense strand
  Description of Artificial SequenceInfluenza A virus RNA polymerase (PA) target siRNA PA 2256 sense strand
gaagaucuguuccaccauutt

SEQ ID NO: 77 - DNA, 21 bp, Artificial Sequence
  Description of Combined DNA/RNA Molecule Influenza A virusRNA polymerase (PA) target siRNA PA 2256 antisense strand
  Description of Artificial SequenceInfluenza A virus RNApolymerase (PA) target siRNA PA 2256 antisense strand
aaugguggaacagaucuuctt

SEQ ID NO: 78 - DNA, 21 bp, Artificial Sequence
  Description of Combined DNA/RNA Molecule Influenza A virusRNA polymerase (PA) target siRNA PA 2087 sense strand
  Description of Artificial SequenceInfluenza A virus RNApolymerase (PA) target siRNA PA 2087 sense strand
gcaauugaggagugccugatt

SEQ ID NO: 79 - DNA, 21 bp, Artificial Sequence
  Description of Combined DNA/RNA Molecule Influenza A virus RNA polymerase (PA) target siRNA PA 2087 antisense strand
  Description of Artificial SequenceInfluenza A virus RNApolymerase (PA) target siRNA PA 2087 antisense strand
ucaggcacuccucaauugctt

SEQ ID NO: 80 - RNA, 21 nt, Artificial Sequence
  Description of Artificial Sequencecontrol target ApoB siRNA sense strand
gucaucacacugaauaccaau

SEQ ID NO: 81 - RNA, 23 nt, Artificial Sequence
  Description of Artificial Sequencecontrol target ApoB siRNA antisense strand
auugguauucagugugaugacac

SEQ ID NO: 82 - RNA, 21 nt, Artificial Sequence
  Description of Artificial Sequencecontrol target luciferase (Luc) siRNA sense strand
gauuauguccgguuauguauu

SEQ ID NO: 83 - RNA, 21 nt, Artificial Sequence
  Description of Artificial Sequencecontrol target luciferase (Luc) siRNA antisense strand
uacauaaccggacauaaucuu

SEQ ID NO: 84 - RNA, 21 nt, Artificial Sequence
  Description of Artificial Sequencecontrol target scrambled luciferase (Luc scr) siRNA sense strand
auguauuggccuguauuaguu

SEQ ID NO: 85 - RNA, 21 nt, Artificial Sequence
  Description of Artificial Sequencecontrol target scrambled luciferase (Luc scr) siRNA antisense strand
cuaauacaggccaauacauuu

SEQ ID NO: 86 - RNA, 21 nt, Artificial Sequence
  Description of Artificial SequenceInfluenza A virusnucleocapsid protein (NP) target siRNA NP 97 sense strand
acgccagaaugccacugaauu

SEQ ID NO: 87 - RNA, 21 nt, Artificial Sequence
  Description of Artificial SequenceInfluenza A virusnucleocapsid protein (NP) target siRNA NP 97 antisense strand
uucaguggcauucuggcguuu

SEQ ID NO: 88 - DNA, 21 bp, Artificial Sequence
  Description of Combined DNA/RNA Molecule Influenza A virusnucleocapsid protein (NP) target siRNA 2′OMe- modified NP 97 U2/0 sense strand
  Description of Artificial SequenceInfluenza A virus nucleocapsid protein (NP) target siRNA 2′OMe-modified NP 97 U2/0 sense strand
  modified_base (1)...(21): n = 2′-O-methyl uridine
acgccagaangccacngaatt

SEQ ID NO: 89 - DNA, 21 bp, Artificial Sequence
  Description of Combined DNA/RNA Molecule Influenza A virusnucleocapsid protein (NP) target siRNA NP 97 U2/0antisense strand
  Description of Artificial SequenceInfluenza A virusnucleocapsid protein (NP) target siRNA NP 97 U2/0 antisense strand
uucaguggcauucuggcgutt

SEQ ID NO: 90 - RNA, 21 nt, Artificial Sequence
  Description of Artificial SequenceInfluenza A virusnucleocapsid protein (NP) target siRNA NP 165 sense strand
uccaaaugugcacagaacuuu

SEQ ID NO: 91 - RNA, 21 nt, Artificial Sequence
  Description of Artificial SequenceInfluenza A virus nucleocapsid protein (NP) target siRNA NP 165 antisense strand
aguucugugcacauuuggauu

SEQ ID NO: 92 - DNA, 21 bp, Artificial Sequence
  Description of Combined DNA/RNA Molecule Influenza A virusnucleocapsid protein (NP) target siRNA 2′OMe- modified NP 165 U4/0 sense strand
  Description of Artificial SequenceInfluenza A virusnucleocapsid protein (NP) target siRNA 2′OMe-modified NP 165 U4/0 sense strand
  modified_base (1)...(21): n = 2′-O-methyl uridine
nccaaangngcacagaacntt

SEQ ID NO: 93 - DNA, 21 bp, Artificial Sequence
  Description of Combined DNA/RNA Molecule Influenza A virusnucleocapsid protein (NP) target siRNA NP 165 U4/0 antisense strand
  Description of Artificial SequenceInfluenza A virusnucleocapsid protein (NP) target siRNA NP 165 U4/0 antisense strand
aguucugugcacauuuggatt

SEQ ID NO: 94 - RNA, 21 nt, Artificial Sequence
  Description of Artificial SequenceInfluenza A virus nucleocapsid protein (NP) target siRNA NP 171 sense strand
ugugcacagaacuuaaacuuu

SEQ ID NO: 95 - RNA, 21 nt, Artificial Sequence
  Description of Artificial SequenceInfluenza A virus nucleocapsid protein (NP) target siRNA NP 171 antisense strand
aguuuaaguucugugcacauu

SEQ ID NO: 96 - DNA, 21 bp, Artificial Sequence
  Description of Combined DNA/RNA Molecule Influenza A virusnucleocapsid protein (NP) target siRNA 2′OMe- modified NP 171 U5/0 sense strand
  Description of Artificial SequenceInfluenza A virusnucleocapsid protein (NP) target siRNA 2′OMe-modified NP 171 U5/0 sense strand
  modified_base (1)...(21): n = 2′-O-methyl uridine
ngngcacagaacnnaaacntt

SEQ ID NO: 97 - DNA, 21 bp, Artificial Sequence
  Description of Combined DNA/RNA Molecule Influenza A virusnucleocapsid protein (NP) target siRNA NP 171 U5/0 antisense strand
  Description of Artificial SequenceInfluenza A virusnucleocapsid protein (NP) target siRNA NP 171 U5/0 antisense strand
aguuuaaguucugugcacatt

SEQ ID NO: 98 - RNA, 21 nt, Artificial Sequence
  Description of Artificial SequenceInfluenza A virus nucleocapsid protein (NP) target siRNA NP 222 sense strand
gcuuaacaauagagagaauuu

SEQ ID NO: 99 - RNA, 21 nt, Artificial Sequence
  Description of Artificial SequenceInfluenza A virusnucleocapsid protein (NP) target siRNA NP 222 antisense strand
auucucucuauuguuaagcuu

SEQ ID NO: 100 - DNA, 21 bp, Artificial Sequence
  Description of Combined DNA/RNA Molecule Influenza A virusnucleocapsid protein (NP) target siRNA 2′OMe- modified NP 222 U4/0 sense strand
  Description of Artificial SequenceInfluenza A virusnucleocapsid protein (NP) target siRNA 2′OMe-modified NP 222 U4/0 sense strand
  modified_base (1)...(21): n = 2′-O-methyl uridine
gcnnaacaanagagagaantt

SEQ ID NO: 101 - DNA, 21 bp, Artificial Sequence
  Description of Combined DNA/RNA Molecule Influenza A virusnucleocapsid protein (NP) target siRNA NP 222 U4/0 antisense strand
  Description of Artificial SequenceInfluenza A virus nucleocapsid protein (NP) target siRNA NP 222 U4/0 antisense strand
auucucucuauuguuaagctt

SEQ ID NO: 102 - RNA, 21 nt, Artificial Sequence
  Description of Artificial SequenceInfluenza A virus nucleocapsid protein (NP) target siRNA NP 383 sense strand
gaagaaauaaggcgaaucuuu

SEQ ID NO: 103 - RNA, 21 nt, Artificial Sequence
  Description of Artificial SequenceInfluenza A virusnucleocapsid protein (NP) target siRNA NP 383 antisense strand
agauucgccuuauuucuucuu

SEQ ID NO: 104 - DNA, 21 bp, Artificial Sequence
  Description of Combined DNA/RNA Molecule Influenza A virusnucleocapsid protein (NP) target siRNA 2′OMe- modified NP 383 U3/0 sense strand
  Description of Artificial Sequence Influenza A virusnucleocapsid protein (NP) target siRNA 2′OMe- modifiedNP 383 U3/0 sense strand
  modified_base (1)...(21): n = 2′-O-methyl uridine
gaagaaanaaggcgaancntt

SEQ ID NO: 105 - DNA, 21 bp, Artificial Sequence
  Description of Combined DNA/RNA Molecule Influenza A virusnucleocapsid protein (NP) target siRNA NP 383 U3/0 antisense strand
  Description of Artificial SequenceInfluenza A virusnucleocapsid protein (NP) target siRNA NP 383 U3/0 antisense strand
agauucgccuuauuucuuctt

SEQ ID NO: 106 - DNA, 21 bp, Artificial Sequence
  Description of Combined DNA/RNA Molecule Influenza A virusnucleocapsid protein (NP) target siRNA 2′OMe- modified NP 411 U5/0 sense strand
  Description of Artificial SequenceInfluenza A virusnucleocapsid protein (NP) target siRNA 2′OMe-modified NP 411 U5/0 sense strand
  modified_base (1)...(21): n = 2′-O-methyl uridine
agcnaanaanggngacgantt

SEQ ID NO: 107 - DNA, 21 bp, Artificial Sequence
  Description of Combined DNA/RNA Molecule Influenza A virusnucleocapsid protein (NP) target siRNA NP 411 U5/0 antisense strand
  Description of Artificial SequenceInfluenza A virusnucleocapsid protein (NP) target siRNA NP 411 U5/0 antisense strand
aucgucaccauuauuagcutt

SEQ ID NO: 108 - RNA, 21 nt, Artificial Sequence
  Description of Artificial SequenceInfluenza A virusnucleocapsid protein (NP) target siRNA NP 724 sense strand
agggaaauuucaaacugcuuu

SEQ ID NO: 109 - RNA, 21 nt, Artificial Sequence
  Description of Artificial SequenceInfluenza A virusnucleocapsid protein (NP) target siRNA NP 724 antisense strand
agcaguuugaaauuucccuuu

SEQ ID NO: 110 - DNA, 21 bp, Artificial Sequence
  Description of Combined DNA/RNA Molecule Influenza A virusnucleocapsid protein (NP) target siRNA 2′OMe- modified NP 724 U5/0 sense strand
  Description of Artificial SequenceInfluenza A virus nucleocapsid protein (NP) target siRNA 2′OMe-modified NP 724 U5/0 sense strand
  modified_base (1)...(21): n = 2′-O-methyl uridine
agggaaannncaaacngcntt

SEQ ID NO: 111 - DNA, 21 bp, Artificial Sequence
  Description of Combined DNA/RNA Molecule Influenza A virusnucleocapsid protein (NP) target siRNA NP 724 U5/0 antisense strand
  Description of Artificial SequenceInfluenza A virusnucleocapsid protein (NP) target siRNA NP 724 U5/0 antisense strand
agcaguuugaaauuucccutt

SEQ ID NO: 112 - DNA, 21 bp, Artificial Sequence
  Description of Combined DNA/RNA Molecule Influenza A virusnucleocapsid protein (NP) target siRNA 2′OMe- modified NP 929 U6/0 sense strand
  Description of Artificial SequenceInfluenza A virus nucleocapsid protein (NP) target siRNA 2′OMe-modified NP 929 U6/0 sense strand
  modified_base (1)...(21): n = 2′-O-methyl uridine
ganacncncnagncggaantt

SEQ ID NO: 113 - DNA, 21 bp, Artificial Sequence
  Description of Combined DNA/RNA Molecule Influenza A virusnucleocapsid protein (NP) target siRNA NP 929 U6/0 antisense strand
  Description of Artificial SequenceInfluenza A virus nucleocapsid protein (NP) target siRNA NP 929 U6/0 antisense strand
auuccgacuagagaguauctt

SEQ ID NO: 114 - RNA, 21 nt, Artificial Sequence
  Description of Artificial SequenceInfluenza A virusnucleocapsid protein (NP) target siRNA NP 1000 sense strand
ugagaauccagcacacaaguu

SEQ ID NO: 115 - RNA, 21 nt, Artificial Sequence
  Description of Artificial SequenceInfluenza A virus nucleocapsid protein (NP) target siRNA NP 1000 antisense strand
cuugugugcuggauucucauu

SEQ ID NO: 116 - DNA, 21 bp, Artificial Sequence
  Description of Combined DNA/RNA Molecule Influenza A virusnucleocapsid protein (NP) target siRNA 2′OMe- modified NP 1000 U2/0 sense strand
  Description of Artificial SequenceInfluenza A virus nucleocapsid protein (NP) target siRNA 2′OMe-modified NP 1000 U2/0 sense strand
  modified_base (1)...(21): n = 2′-O-methyl uridine
ngagaanccagcacacaagtt

SEQ ID NO: 117 - DNA, 21 bp, Artificial Sequence
  Description of Combined DNA/RNA Molecule Influenza A virusnucleocapsid protein (NP) target siRNA NP 1000 U2/0 antisense strand
  Description of Artificial SequenceInfluenza A virusnucleocapsid protein (NP) target siRNA NP 1000 U2/0 antisense strand
cuugugugcuggauucucatt

SEQ ID NO: 118 - RNA, 21 nt, Artificial Sequence
  Description of Artificial SequenceInfluenza A virus nucleocapsid protein (NP) target siRNA NP 1096 sense strand
gguggucccaagagggaaguu

SEQ ID NO: 119 - RNA, 21 nt, Artificial Sequence
  Description of Artificial SequenceInfluenza A virus nucleocapsid protein (NP) target siRNA NP 1096 antisense strand
cuucccucuugggaccaccuu

SEQ ID NO: 120 - DNA, 21 bp, Artificial Sequence
  Description of Combined DNA/RNA Molecule Influenza A virusnucleocapsid protein (NP) target siRNA 2′OMe- modified NP 1096 U2/0 sense strand
  Description of Artificial SequenceInfluenza A virusnucleocapsid protein (NP) target siRNA 2′OMe-modified NP 1096 U2/0 sense strand
  modified_base (1)...(21): n = 2′-O-methyl uridine
ggnggncccaagagggaagtt

SEQ ID NO: 121 - DNA, 21 bp, Artificial Sequence
  Description of Combined DNA/RNA Molecule Influenza A virusnucleocapsid protein (NP) target siRNA NP 1096 U2/0 antisense strand
  Description of Artificial SequenceInfluenza A virus nucleocapsid protein (NP) target siRNA NP 1096 U2/0 antisense strand
cuucccucuugggaccacctt

SEQ ID NO: 122 - DNA, 21 bp, Artificial Sequence
  Description of Combined DNA/RNA Molecule Influenza A virusnucleocapsid protein (NP) target siRNA 2′OMe- modified NP 1116 U5/0 sense strand
  Description of Artificial SequenceInfluenza A virusnucleocapsid protein (NP) target siRNA 2′OMe-modified NP 1116 U5/0 sense strand
  modified_base (1)...(21): n = 2′-O-methyl uridine
gcnunccacnagaggagnntt

SEQ ID NO: 123 - DNA, 21 bp, Artificial Sequence
  Description of Combined DNA/RNA Molecule Influenza A virusnucleocapsid protein (NP) target siRNA NP 1116 U5/0 antisense strand
  Description of Artificial SequenceInfluenza A virusnucleocapsid protein (NP) target siRNA NP 1116 U5/0 antisense strand
aacuccucuaguggaaagctt

SEQ ID NO: 124 - RNA, 21 nt, Artificial Sequence
  Description of Artificial SequenceInfluenza A virusnucleocapsid protein (NP) target siRNA NP 1320 sense strand
uggcagcauucacugggaauu

SEQ ID NO: 125 - RNA, 21 nt, Artificial Sequence
  Description of Artificial SequenceInfluenza A virus nucleocapsid protein (NP) target siRNA NP 1320 antisense strand
uucccagugaaugcugccauu

SEQ ID NO: 126 - DNA, 21 bp, Artificial Sequence
  Description of Combined DNA/RNA Molecule Influenza A virusnucleocapsid protein (NP) target siRNA 2′OMe- modified NP 1320 U4/0 sense strand
  Description of Artificial SequenceInfluenza A virus nucleocapsid protein (NP) target siRNA 2′OMe-modified NP 1320 U4/0 sense strand
  modified_base (1)...(21): n = 2′O-methyl uridine
nggcagcanncacngggaatt

SEQ ID NO: 127 - DNA, 21 bp, Artificial Sequence
  Description of Combined DNA/RNA Molecule Influenza A virusnucleocapsid protein (NP) target siRNA NP 1320 U4/0 antisense strand
  Description of Artificial SequenceInfluenza A virus nucleocapsid protein (NP) target siRNA NP 1320 U4/0 antisense strand
uucccagugaaugcugccatt

SEQ ID NO: 128 - RNA, 21 nt, Artificial Sequence
  Description of Artificial SequenceInfluenza A virus nucleocapsid protein (NP) target siRNA NP 1485 sense strand
ugaguaaugaaggaucuuauu

SEQ ID NO: 129 - RNA, 21 nt, Artificial Sequence
  Description of Artificial SequenceInfluenza A virusnucleocapsid protein (NP) target siRNA NP 1485 antisense strand
uaagauccuucauuacucauu

SEQ ID NO: 130 - DNA, 21 bp, Artificial Sequence
  Description of Combined DNA/RNA Molecule Influenza A virusnucleocapsid protein (NP) target siRNA 2′OMe- modified NP 1485 U6/0 sense strand
  Description of Artificial SequenceInfluenza A virus nucleocapsid protein (NP) target siRNA 2′OMe-modified NP 1485 U6/0 sense strand
  modified_base (1)...(21): n = 2′-O-methyl uridine
ngagnaangaaggancnnatt

SEQ ID NO: 131 - DNA, 21 bp, Artificial Sequence
  Description of Combined DNA/RNA Molecule Influenza A virusnucleocapsid protein (NP) target siRNA NP 1485 U6/0 antisense strand
  Description of Artificial SequenceInfluenza A virus nucleocapsid protein (NP) target siRNA NP 1485 U6/0 antisense strand
uaagauccuucauuacucatt

SEQ ID NO: 132 - DNA, 21 bp, Artificial Sequence
  Description of Combined DNA/RNA Molecule Influenza A virusnucleocapsid protein (NP) target siRNA 2′OMe- modified NP 1496 U4/0 sense strand
  Description of Artificial SequenceInfluenza A virusnucleocapsid protein (NP) target siRNA 2′OMe-modified NP 1496 U4/0 sense strand
  modified_base (1)...(21): n = 2′-O-methyl uridine
ggancunaunucnucggagtt

SEQ ID NO: 133 - DNA, 21 bp, Artificial Sequence
  Description of Combined DNA/RNA Molecule Influenza A virusnucleocapsid protein (NP) target siRNA NP 1496 U4/0 antisense strand
  Description of Artificial SequenceInfluenza A virus nucleocapsid protein (NP) target siRNA NP 1496 U4/0 antisense strand
cuccgaagaaauaagaucctt

SEQ ID NO: 134 - DNA, 21 bp, Artificial Sequence
  Description of Combined DNA/RNA Molecule Influenza A virusnucleocapsid protein (NP) target siRNA 2′OMe- modified NP 1496 U8/0 sense strand
  Description of Artificial SequenceInfluenza A virusnucleocapsid protein (NP) target siRNA 2′OMe-modified NP 1496 U8/0 sense strand
  modified_base (1)...(21): n = 2′-O-methyl uridine
ggancnnannncnncggagtt

SEQ ID NO: 135 - DNA, 21 bp, Artificial Sequence
  Description of Combined DNA/RNA Molecule Influenza A virusnucleocapsid protein (NP) target siRNA NP 1496 U8/0 antisense strand
  Description of Artificial Sequence Influenza A virusnucleocapsid protein (NP) target siRNA NP 1496 U8/0 antisense strand
cuccgaagaaauaagaucctt

SEQ ID NO: 136 - DNA, 21 bp, Artificial Sequence
  Description of Combined DNA/RNA Molecule Influenza A virusRNA polymerase (PA) target siRNA PA 194 sense strand
  Description of Artificial SequenceInfluenza A virus RNApolymerase (PA) target siRNA PA 194 sense strand
ggcgagucaauaaucguagtt

SEQ ID NO: 137 - DNA, 21 bp, Artificial Sequence
  Description of Combined DNA/RNA Molecule Influenza A virusRNA polymerase (PA) target siRNA PA 194 antisense strand
  Description of Artificial SequenceInfluenza A virus RNA polymerase (PA) target siRNA PA 194 antisense strand
cuacgauuauugacucgcctt

SEQ ID NO: 138 - DNA, 21 bp, Artificial Sequence
  Description of Combined DNA/RNA Molecule Influenza A virusRNA polymerase (PA) target siRNA 2′OMe-modified PA 194 U4/0 sense strand
  Description of Artificial SequenceInfluenza A virus RNA polymerase (PA) target siRNA 2′OMe-modified PA 194 U4/0 sense strand
  modified_base (1)...(21): n = 2′-O-methyl uridine
ggcgagncaanaancgnagtt

SEQ ID NO: 139 - DNA, 21 bp, Artificial Sequence
  Description of Combined DNA/RNA Molecule Influenza A virusRNA polymerase (PA) target siRNA PA 194 U4/0 antisensestrand
  Description of Artificial SequenceInfluenza A virus RNApolymerase (PA) target siRNA PA 194 U4/0 antisense strand
cuacgauuauugacucgcctt

SEQ ID NO: 140 - DNA, 21 bp, Artificial Sequence
  Description of Combined DNA/RNA Molecule Influenza A virusRNA polymerase (PA) target siRNA PA 212 sense strand
  Description of Artificial SequenceInfluenza A virus RNA polymerase (PA) target siRNA PA 212 sense strand
gaacuuggugauccuaaugtt

SEQ ID NO: 141 - DNA, 21 bp, Artificial Sequence
  Description of Combined DNA/RNA Molecule Influenza A virusRNA polymerase (PA) target siRNA PA 212 antisense strand
  Description of Artificial SequenceInfluenza A virus RNA polymerase (PA) target siRNA PA 212 antisense strand
cauuaggaucaccaaguuctt

SEQ ID NO: 142 - DNA, 21 bp, Artificial Sequence
  Description of Combined DNA/RNA Molecule Influenza A virusRNA polymerase (PA) target siRNA 2′OMe-modified PA 212 U6/0 sense strand
  Description of Artificial SequenceInfluenza A virus RNApolymerase (PA) target siRNA 2′OMe-modified PA 212 U6/0 sense strand
  modified_base (1)...(21): n = 2′-O-methyl uridine
gaacnnggnganccnaangtt

SEQ ID NO: 143 - DNA, 21 bp, Artificial Sequence
  Description of Combined DNA/RNA Molecule Influenza A virusRNA polymerase (PA) target siRNA PA 212 U6/0 antisensestrand
  Description of Artificial SequenceInfluenza A virus RNApolymerase (PA) target siRNA PA 212 U6/0 antisense strand
cauuaggaucaccaaguuctt

SEQ ID NO: 144 - DNA, 21 bp, Artificial Sequence
  Description of Combined DNA/RNA Molecule Influenza A virusRNA polymerase (PA) target siRNA PA 392 sense strand
  Description of Artificial SequenceInfluenza A virus RNApolymerase (PA) target siRNA PA 392 sense strand
aggagagaaguucacauautt

SEQ ID NO: 145 - DNA, 21 bp, Artificial Sequence
  Description of Combined DNA/RNA Molecule Influenza A virusRNA polymerase (PA) target siRNA PA 392 antisense strand
  Description of Artificial SequenceInfluenza A virus RNApolymerase (PA) target siRNA PA 392 antisense strand
auaugugaacuucucuccutt

SEQ ID NO: 146 - DNA, 21 bp, Artificial Sequence
  Description of Combined DNA/RNA Molecule Influenza A virusRNA polymerase (PA) target siRNA 2′OMe-modified PA 392 U4/0 sense strand
  Description of Artificial SequenceInfluenza A virus RNA polymerase (PA) target siRNA 2′OMe-modified PA 392 U4/0 sense strand
  modified_base (1)...(21): n = 2′-O-methyl uridine
aggagagaagnncacanantt

SEQ ID NO: 147 - DNA, 21 bp, Artificial Sequence
  Description of Combined DNA/RNA Molecule Influenza A virusRNA polymerase (PA) target siRNA PA 392 U4/0 antisensestrand
  Description of Artificial SequenceInfluenza A virus RNApolymerase (PA) target siRNA PA 392 U4/0 antisense strand
auaugugaacuucucuccutt

SEQ ID NO: 148 - DNA, 21 bp, Artificial Sequence
  Description of Combined DNA/RNA Molecule Influenza A virusRNA polymerase (PA) target siRNA PA 751 sense strand
  Description of Artificial Sequence Influenza A virus RNApolymerase (PA) target siRNA PA 751 sense strand
gggcaagcugucucaaaugtt

SEQ ID NO: 149 - DNA, 21 bp, Artificial Sequence
  Description of Combined DNA/RNA Molecule Influenza A virusRNA polymerase (PA) target siRNA PA 751 antisense strand
  Description of Artificial SequenceInfluenza A virus RNA polymerase (PA) target siRNA PA 751 antisense strand
cauuugagacagcuugccctt

SEQ ID NO: 150 - DNA, 21 bp, Artificial Sequence
  Description of Combined DNA/RNA Molecule Influenza A virusRNA polymerase (PA) target siRNA 2′OMe-modified PA 751 U4/0 sense strand
  Description of Artificial SequenceInfluenza A virus RNA polymerase (PA) target siRNA 2′OMe-modified PA 751 U4/0 sense strand
  modified_base (1)...(21): n = 2′-O-methyl uridine
gggcaagcngncncaaangtt

SEQ ID NO: 151 - DNA, 21 bp, Artificial Sequence
  Description of Combined DNA/RNA Molecule Influenza A virusRNA polymerase (PA) target siRNA PA 751 U4/0 antisensestrand
  Description of Artificial SequenceInfluenza A virus RNA polymerase (PA) target siRNA PA 751 U4/0 antisense strand
cauuugagacagcuugccctt

SEQ ID NO: 152 - DNA, 21 bp, Artificial Sequence
  Description of Combined DNA/RNA Molecule Influenza A virusRNA polymerase (PA) target siRNA PA 783 sense strand
  Description of Artificial SequenceInfluenza A virus RNA polymerase (PA) target siRNA PA 783 sense strand
augcuagaauugaaccuuutt

SEQ ID NO: 153 - DNA, 21 bp, Artificial Sequence
  Description of Combined DNA/RNA Molecule Influenza A virusRNA polymerase (PA) target siRNA PA 783 antisense strand
  Description of Artificial SequenceInfluenza A virus RNApolymerase (PA) target siRNA PA 783 antisense strand
aaagguucaauucuagcautt

SEQ ID NO: 154 - DNA, 21 bp, Artificial Sequence
  Description of Combined DNA/RNA Molecule Influenza A virusRNA polymerase (PA) target siRNA 2′OMe-modified PA 783 U7/0 sense strand
  Description of Artificial SequenceInfluenza A virus RNApolymerase (PA) target siRNA 2′OMe-modified PA 783 U7/0 sense strand
  modified_base (1)...(21): n = 2′-O-methyl uridine
angcnagaanngaaccnnntt

SEQ ID NO: 155 - DNA, 21 bp, Artificial Sequence
  Description of Combined DNA/RNA Molecule Influenza A virusRNA polymerase (PA) target siRNA PA 783 U7/0 antisensestrand
  Description of Artificial SequenceInfluenza A virus RNApolymerase (PA) target siRNA PA 783 U7/0 antisense strand
aaagguucaauucuagcautt

SEQ ID NO: 156 - DNA, 21 bp, Artificial Sequence
  Description of Combined DNA/RNA Molecule Influenza A virusRNA polymerase (PA) target siRNA PA 813 sense strand
  Description of Artificial SequenceInfluenza A virus RNA polymerase (PA) target siRNA PA 813 sense strand
caccacgaccacuuagacutt

SEQ ID NO: 157 - DNA, 21 bp, Artificial Sequence
  Description of Combined DNA/RNA Molecule Influenza A virusRNA polymerase (PA) target siRNA PA 813 antisense strand
  Description of Artificial SequenceInfluenza A virus RNApolymerase (PA) target siRNA PA 813 antisense strand
agucuaaguggucguggugtt

SEQ ID NO: 158 - DNA, 21 bp, Artificial Sequence
  Description of Combined DNA/RNA Molecule Influenza A virusRNA polymerase (PA) target siRNA 2′OMe-modified PA 813 U3/0 sense strand
  Description of Artificial SequenceInfluenza A virus RNApolymerase (PA) target siRNA 2′OMe-modified PA 813 U3/0 sense strand
  modified_base (1)...(21): n = 2′-O-methyl uridine
caccacgaccacnnagacntt

SEQ ID NO: 159 - DNA, 21 bp, Artificial Sequence
  Description of Combined DNA/RNA Molecule Influenza A virusRNA polymerase (PA) target siRNA PA 813 U3/0 antisensestrand
  Description of Artificial SequenceInfluenza A virus RNApolymerase (PA) target siRNA PA 813 U3/0 antisense strand
agucuaaguggucguggugtt

SEQ ID NO: 160 - DNA, 21 bp, Artificial Sequence
  Description of Combined DNA/RNA Molecule Influenza A virusRNA polymerase (PA) target siRNA PA 1656 sense strand
  Description of Artificial Sequence Influenza A virus RNApolymerase (PA) target siRNA PA 1656 sense strand
uaggagauaugcuucuaagtt

SEQ ID NO: 161 - DNA, 21 bp, Artificial Sequence
  Description of Combined DNA/RNA Molecule Influenza A virusRNA polymerase (PA) target siRNA PA 1656 antisense strand
  Description of Artificial SequenceInfluenza A virus RNA polymerase (PA) target siRNA PA 1656 antisense strand
cuuagaagcauaucuccuatt

SEQ ID NO: 162 - DNA, 21 bp, Artificial Sequence
  Description of Combined DNA/RNA Molecule Influenza A virusRNA polymerase (PA) target siRNA 2′OMe-modified PA 1656 U6/0 sense strand
  Description of Artificial Sequence Influenza A virus RNApolymerase (PA) target siRNA 2′OMe-modified PA 1656 U6/0 sense strand
  modified_base (1)...(21): n = 2′-O-methyl uridine
naggaganangcnncnaagtt

SEQ ID NO: 163 - DNA, 21 bp, Artificial Sequence
  Description of Combined DNA/RNA Molecule Influenza A virusRNA polymerase (PA) target siRNA PA 1656 U6/0 antisensestrand
  Description of Artificial SequenceInfluenza A virus RNA polymerase (PA) target siRNA PA 1656 U6/0 antisense strand
cuuagaagcauaucuccuatt

SEQ ID NO: 164 - DNA, 21 bp, Artificial Sequence
  Description of Combined DNA/RNA Molecule Influenza A virusRNA polymerase (PA) target siRNA PA 1658 sense strand
  Description of Artificial SequenceInfluenza A virus RNA polymerase (PA) target siRNA PA 1658 sense strand
ggagauaugcuucuaagaatt

SEQ ID NO: 165 - DNA, 21 bp, Artificial Sequence
  Description of Combined DNA/RNA Molecule Influenza A virusRNA polymerase (PA) target siRNA PA 1658 antisense strand
  Description of Artificial SequenceInfluenza A virus RNApolymerase (PA) target siRNA PA 1658 antisense strand
uucuuagaagcauaucucctt

SEQ ID NO: 166 - DNA, 21 bp, Artificial Sequence
  Description of Combined DNA/RNA Molecule Influenza A virusRNA polymerase (PA) target siRNA 2′OMe-modified PA 1658 U5/0 sense strand
  Description of Artificial SequenceInfluenza A virus RNApolymerase (PA) target siRNA 2′OMe-modified PA 1658 U5/0 sense strand
  modified_base (1)...(21): n = 2′-O-methyl uridine
ggaganangcnncnaagaatt

SEQ ID NO: 167 - DNA, 21 bp, Artificial Sequence
  Description of Combined DNA/RNA Molecule Influenza A virusRNA polymerase (PA) target siRNA PA 1658 U5/0 antisensestrand
  Description of Artificial SequenceInfluenza A virus RNApolymerase (PA) target siRNA PA 1658 U5/0 antisense strand
uucuuagaagcauaucucctt

SEQ ID NO: 168 - DNA, 21 bp, Artificial Sequence
  Description of Combined DNA/RNA Molecule Influenza A virusRNA polymerase (PA) target siRNA PA 1884 sense strand
  Description of Artificial SequenceInfluenza A virus RNApolymerase (PA) target siRNA PA 1884 sense strand
uuggagagucucccaaaggtt

SEQ ID NO: 169 - DNA, 21 bp, Artificial Sequence
  Description of Combined DNA/RNA Molecule Influenza A virusRNA polymerase (PA) target siRNA PA 1884 antisense strand
  Description of Artificial SequenceInfluenza A virus RNApolymerase (PA) target siRNA PA 1884 antisense strand
ccuuugggagacucuccaatt

SEQ ID NO: 170 - DNA, 21 bp, Artificial Sequence
  Description of Combined DNA/RNA Molecule Influenza A virusRNA polymerase (PA) target siRNA 2′OMe-modified PA 1884 U4/0 sense strand
  Description of Artificial SequenceInfluenza A virus RNA polymerase (PA) target siRNA 2′OMe-modified PA 1884 U4/0 sense strand
  modified_base (1)...(21): n = 2′-O-methyl uridine
nnggagagncncccaaaggtt

SEQ ID NO: 171 - DNA, 21 bp, Artificial Sequence
  Description of Combined DNA/RNA Molecule Influenza A virusRNA polymerase (PA) target siRNA PA 1884 U4/0 antisensestrand
  Description of Artificial SequenceInfluenza A virus RNA polymerase (PA) target siRNA PA 1884 U4/0 antisense strand
ccuuugggagacucuccaatt

SEQ ID NO: 172 - DNA, 21 bp, Artificial Sequence
  Description of Combined DNA/RNA Molecule Influenza A virusRNA polymerase (PA) target siRNA PA 2098 sense strand
  Description of Artificial SequenceInfluenza A virus RNA polymerase (PA) target siRNA PA 2098 sense strand
gugccuaauuaaugauccctt

SEQ ID NO: 173 - DNA, 21 bp, Artificial Sequence
  Description of Combined DNA/RNA Molecule Influenza A virusRNA polymerase (PA) target siRNA PA 2098 antisense strand
  Description of Artificial SequenceInfluenza A virus RNApolymerase (PA) target siRNA PA 2098 antisense strand
gggaucauuaauuaggcactt

SEQ ID NO: 174 - DNA, 21 bp, Artificial Sequence
  Description of Combined DNA/RNA Molecule Influenza A virusRNA polymerase (PA) target siRNA 2′OMe-modified PA 2098 U6/0 sense strand
  Description of Artificial SequenceInfluenza A virus RNA polymerase (PA) target siRNA 2′OMe-modified PA 2098 U6/0 sense strand
  modified_base (1)...(21): n = 2′-O-methyl uridine
gngccnaannaanganccctt

SEQ ID NO: 175 - DNA, 21 bp, Artificial Sequence
  Description of Combined DNA/RNA Molecule Influenza A virusRNA polymerase (PA) target siRNA PA 2098 U6/0 antisensestrand
  Description of Artificial SequenceInfluenza A virus RNApolymerase (PA) target siRNA PA 2098 U6/0 antisense strand
gggaucauuaauuaggcactt

SEQ ID NO: 176 - DNA, 21 bp, Artificial Sequence
  Description of Artificial Sequencecontrol target 2′OMe-modified ApoB mismatch (mm) siRNA sense strand
  modified_base (1)...(21): n = 2′-O-methyl uridine
gngancagacncaanacgaau

SEQ ID NO: 177 - DNA, 23 bp, Artificial Sequence
  Description of Artificial Sequencecontrol target 2′OMe-modified ApoB mismatch (mm) siRNA antisense strand
  modified_base (11)...(11): n = 2′-O-methyl cytosine
  modified_base (12)...(12): n = 2′-O-methyl uridine
  modified_base (13)...(13): n = 2′-O-methyl guanosine
  modified_base (14)...(14): n = 2′-O-methyl adenosine
auucguauugnnnnugaucacac

(19) United States
(12) Patent Application Publication
(10) Pub. No.: US 2007/0218122 A1
(43) Pub. Date: Sep. 20, 2007

(54) siRNA silencing of influenza virus gene expression

(75) Inventors: Ian MacLachlan, Mission (CA); Marjorie Robbins, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Protiva Biotherapeutics, Inc., Burnaby (CA)

(21) Appl. No.: 11/601,437

(22) Filed: Nov. 17, 2006

Related U.S. Application Data
(60) Provisional application No. 60/737,945, filed on Nov. 18, 2005.

(51) Int. Cl. A61K 48/00; A61K 9/127; C07H 21/02

(52) U.S. Cl. 424/450; 514/44; 536/23.1; 977/907

(57) ABSTRACT

The present invention provides siRNA molecules that target influenza virus gene expression and methods of using such siRNA molecules to silence influenza virus gene expression. The present invention also provides nucleic acid-lipid particles that target influenza virus gene expression comprising an siRNA that silences influenza virus gene expression, a cationic lipid, and a non-cationic lipid.

### CROSS-REFERENCES TO RELATED APPLICATIONS

The present application claims priority to U.S. Provisional Application No. 60/737,945, filed Nov. 18, 2005, the disclosure of which is herein incorporated by reference in its entirety for all purposes.

### BACKGROUND OF THE INVENTION

The flu is a contagious respiratory illness caused by influenza viruses. Flu patients typically exhibit high fever, headache, extreme tiredness, dry cough, sore throat, nasal congestion, and muscle aches. Some flu patients also suffer from gastrointestinal symptoms, such as nausea, vomiting, and diarrhea. Flu infection can also lead to many complications including bacterial pneumonia, dehydration, and worsening of chronic medical conditions, such as congestive heart failure, asthma, diabetes, and ear infections. It can cause mild to severe illness, and at times can lead to death.

Flu includes avian influenza, which is an infectious disease of birds caused by type A strains of the influenza virus. Avian influenza can also be transmitted from birds to humans. To date, all outbreaks of highly pathogenic avian influenza have been caused by influenza A viruses of subtypes H5 and H7. Of the 15 avian influenza virus subtypes, H5N1 is of particular concern. H5N1 mutates rapidly and has a documented propensity to acquire genes from viruses infecting other animal species. H5N1 variants have demonstrated a capacity to directly infect humans in 1997, in Hong Kong in 2003, and in Vietnam in 2004.

Influenza pandemics occur three to four times each century when new virus subtypes emerge and are transmitted from person to person. However, the occurrence of influenza pandemics is unpredictable. In the 20th century, the influenza pandemic of 1918-1919 caused an estimated 40 to 50 million deaths worldwide and was followed by pandemics in 1957-1958 and 1968-1969. It has been estimated that another pandemic could cause over 100 million outpatient visits, more than 25 million hospital admissions, and several million deaths worldwide.

Current efforts to control flu epidemics have focused on vaccination (see, e.g., Wood et al., Nat. Rev. Microbiol., 2:842-847 (2004)). However, due to the rapid mutation rate of the influenza virus, the vaccine formulation must be changed annually and is often not completely effective in preventing influenza (see, e.g., Hay et al., Philos. Trans. R. Soc. Lond. B Biol. Sci., 356:1861-1870 (2001)). Vaccination is also not appropriate for many groups of at-risk individuals and many safety concerns are associated with vaccination (see, e.g., Subbarao et al., Curr. Top. Microbiol. Immunol. 283:313-342 (2004)).

Antiviral drugs, some of which can be used for both treatment and prevention of influenza, are clinically effective against influenza A virus strains, but have serious side-effects including, e.g., anxiety, difficulty concentrating, lightheadedness, delirium, hallucinations, seizures, decreased respiratory function, bronchospasms, bronchitis, cough, sinusitis, nasal infections, headache, diarrhea, nausea, vomiting, and loss of appetite.

Thus, there is a need for compositions and methods for specifically modulating influenza virus gene expression. The present invention addresses these and other needs.

### SUMMARY OF THE INVENTION

The present invention provides siRNA molecules that target influenza virus gene (e.g., PA, PB1, PB2, NP, M1, M2, NS1, and/or NS2) expression and methods of using such siRNA molecules to silence influenza virus (e.g., Influenza A, B, or C virus) gene expression.

In one aspect, the present invention provides an siRNA molecule comprising a double-stranded region of about 15 to about 60 nucleotides in length (e.g., about 15-60, 15-50, 15-40, 15-30, 15-25, or 19-25 nucleotides in length), wherein the siRNA molecule silences expression of an influenza gene selected from the group consisting of PA, PB1, PB2, NP, M1, M2, NS1, and NS2. In certain instances, the siRNA molecule comprises a hairpin loop structure.

In some embodiments, the siRNA has 3′ overhangs of one, two, three, four, or more nucleotides on one or both sides of the double-stranded region. In other embodiments, the siRNA lacks overhangs (i.e., has blunt ends). Preferably, the siRNA has 3′ overhangs of two nucleotides on each side of the double-stranded region. Examples of 3′ overhangs include, but are not limited to, 3′ deoxythymidine (dT) overhangs of one, two, three, four, or more nucleotides.

The siRNA may comprise at least one or a cocktail (e.g., at least two, three, four, five, six, seven, eight, nine, ten, or more) of sequences that silence influenza virus gene expression. In some embodiments, the siRNA comprises at least one or a cocktail of the sequences set forth in Tables 1-4 and 7-8. Preferably, the siRNA comprises at least one or a cocktail of the sequences set forth in Tables 7-8, such as, e.g., unmodified or modified (such as 2′OMe-modified) NP 97, NP 171, NP 222, NP 383, NP 411, NP 929, NP 1116, NP 1485, PA 392, and/or PA 783. In certain instances, the siRNA does not comprise unmodified NP 1496 or PA 2087.

In certain embodiments, the siRNA further comprises a carrier system, e.g., to deliver the siRNA into a cell of a mammal. Examples of carrier systems suitable for use in the present invention include, but are not limited to, nucleic acid-lipid particles, liposomes, micelles, virosomes, nucleic acid complexes, and mixtures thereof. In certain instances, the siRNA is complexed with a lipid such as a cationic lipid to form a lipoplex. In certain other instances, the siRNA is complexed with a polymer such as a cationic polymer (e.g., polyethylenimine (PEI)) to form a polyplex. The siRNA may also be complexed with cyclodextrin or a polymer thereof. Preferably, the siRNA is encapsulated in a nucleic acid-lipid particle.

The present invention also provides a pharmaceutical composition comprising an siRNA described herein and a pharmaceutically acceptable carrier.

In certain embodiments, the siRNA that silences influenza virus gene expression is a modified siRNA in which the double-stranded region comprises at least one, two, three, four, five, six, seven, eight, nine, ten, or more modified nucleotides. Typically, the modified siRNA comprises from about 1% to about 100% (e.g., about 1%, 5%, 10%, 15%, 20%, 25%, 30%, 35%, 40%, 45%, 50%, 55%, 60%, 65%, 70%, 75%, 80%, 85%, 90%, 95%, or 100%) modified nucleotides in the double-stranded region of the siRNA duplex.

In some instances, less than about 20% (e.g., less than about 20%, 15%, 10%, or 5%) or from about 1% to about 20% (e.g., from about 1%-20%, 5%-20%, 10%-20%, or 15%-20%) of the nucleotides in the double-stranded region comprise modified nucleotides. In other instances, at least two, three, four, five, six, seven, eight, nine, ten, or more of the nucleotides in the double-stranded region comprise modified nucleotides selected from the group consisting of modified guanosine nucleotides, modified uridine nucleotides, and mixtures thereof. As a non-limiting example, when one or both strands of the siRNA are selectively modified at uridine and/or guanosine nucleotides, the resulting modified siRNA can comprise less than about 30% modified nucleotides (e.g., less than about 30%, 25%, 20%, 15%, 10%, or 5% modified nucleotides) or from about 1% to about 30% modified nucleotides (e.g., from about 1%-30%, 5%-30%, 10%-30%, 15%-30%, 20%-30%, or 25%-30% modified nucleotides). In yet other instances, at least one, two, three, four, five, six, seven, eight, nine, ten, or more of the nucleotides (e.g., uridine and/or guanosine nucleotides) in the sense strand of the siRNA comprise modified nucleotides and no nucleotides in the antisense strand of the siRNA are modified nucleotides. Advantageously, the modified siRNA is less immunostimulatory than a corresponding unmodified siRNA sequence.

In some embodiments, the modified siRNA comprises modified nucleotides including, but not limited to, 2′OMe nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof.

The modified siRNA can comprise modified nucleotides in one strand (i.e., sense or antisense) or both strands of the double-stranded region of the siRNA. Preferably, uridine and/or guanosine nucleotides are modified at selective positions in the double-stranded region of the siRNA duplex. With regard to uridine nucleotide modifications, at least one, two, three, four, five, six, seven, eight, nine, ten, or more of the uridine nucleotides in the sense and/or antisense strand can be a modified uridine nucleotide (e.g., a 2′OMe-uridine nucleotide). In preferred embodiments, every uridine nucleotide in the sense and/or antisense strand of the double-stranded region of the siRNA comprises modified uridine nucleotides (e.g., 2′OMe-uridine nucleotides). In some embodiments, an siRNA with selective uridine nucleotide modifications can further comprise at least one, two, three, four, five, six, seven, eight, nine, ten, or more modified nucleotides such as, for example, modified guanosine nucleotides, modified adenosine nucleotides, modified cytosine nucleotides, and mixtures thereof. With regard to guanosine nucleotide modifications, at least one, two, three, four, five, six, seven, eight, nine, ten, or more of the guanosine nucleotides in the sense and/or antisense strand can be a modified guanosine nucleotide (e.g., 2′OMe-guanosine nucleotide). In some embodiments, every guanosine nucleotide in the sense and/or antisense strand of the double-stranded region of the siRNA comprises modified guanosine nucleotides (e.g., 2′OMe-guanosine nucleotides). In certain embodiments, an siRNA with selective guanosine nucleotide modifications can further comprise at least one, two, three, four, five, six, seven, eight, nine, ten, or more modified nucleotides such as, for example, modified uridine nucleotides, modified adenosine nucleotides, modified cytosine nucleotides, and mixtures thereof.

In preferred embodiments, the modified siRNA comprises 2′OMe nucleotides (e.g., 2′OMe purine and/or pyrimidine nucleotides) such as, for example, 2′OMe-uridine nucleotides, 2′OMe-guanosine nucleotides, 2′OMe-adenosine nucleotides, 2′OMe-cytosine nucleotides, and mixtures thereof. In certain instances, the modified siRNA comprises 2′OMe-uridine nucleotides, 2′OMe-guanosine nucleotides, or mixtures thereof. In certain other instances, the modified siRNA does not comprise 2′OMe-cytosine nucleotides.

In certain embodiments, the modified siRNA is at least about 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% less immunostimulatory than the corresponding unmodified siRNA sequence. Preferably, the modified siRNA is at least about 80% (e.g., 80%, 85%, 90%, 91%, 92%, 93%, 94%, 95%, 96%, 97%, 98%, 99%, or 100%) less immunostimulatory than the corresponding unmodified siRNA sequence. It will be readily apparent to those of skill in the art that the immunostimulatory properties of the modified siRNA molecule and the corresponding unmodified siRNA molecule can be determined by, for example, measuring INF-α and/or IL-6 levels at about 2-12 hours after systemic administration in a mammal using an appropriate lipid-based delivery system (such as the SNALP delivery system or other lipoplex systems disclosed herein).

In certain other embodiments, the modified siRNA has an IC₅₀ less than or equal to ten-fold that of the corresponding unmodified siRNA (i.e., the modified siRNA has an IC₅₀ that is less than or equal to ten-times the IC₅₀ of the corresponding unmodified siRNA). In some instances, the modified siRNA has an IC₅₀ less than or equal to three-fold that of the corresponding unmodified siRNA. In other instances, the modified siRNA preferably has an IC₅₀ less than or equal to two-fold that of the corresponding unmodified siRNA. It will be readily apparent to those of skill in the art that a dose response curve can be generated and the IC₅₀ values for the modified siRNA and the corresponding unmodified siRNA can be readily determined using methods known to those of skill in the art.

Preferably, the modified siRNA is at least about 80% (e.g., 80%, 85%, 90%, 91%, 92%, 93%, 94%, 95%, 96%, 97%, 98%, 99%, or 100%) less immunostimulatory than the corresponding unmodified siRNA sequence, and the modified siRNA has an IC₅₀ less than or equal to ten-fold (preferably, three-fold and more preferably, two-fold) that of the corresponding unmodified siRNA sequence.

In some embodiments, the modified siRNA is capable of silencing at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, or more of the expression of the target sequence relative to the corresponding unmodified siRNA sequence.

In other embodiments, the corresponding unmodified siRNA sequence comprises at least one, two, three, four, five, six, seven, or more 5′-GU-3′ motifs. The 5′-GU-3′ motif can be in the sense strand, the antisense strand, or both strands of the unmodified siRNA sequence.

In some embodiments, the modified siRNA does not comprise phosphate backbone modifications, e.g., in the sense and/or antisense strand of the double-stranded region. In other embodiments, the modified siRNA does not comprise 2′-deoxy nucleotides, e.g., in the sense and/or antisense strand of the double-stranded region. In certain instances, the nucleotide at the 3′-end of the double-stranded region in the sense and/or antisense strand is not a modified nucleotide. In certain other instances, the nucleotides near the 3′-end (e.g., within one, two, three, or four nucleotides of the 3′-end) of the double-stranded region in the sense and/or antisense strand are not modified nucleotides.

In another aspect, the present invention provides a nucleic acid-lipid particle comprising an siRNA that silences influenza virus gene expression, a cationic lipid, and a non-cationic lipid. In certain instances, the nucleic acid-lipid particle further comprises a conjugated lipid that inhibits aggregation of particles. Preferably, the nucleic acid-lipid particle comprises an siRNA that silences influenza virus gene expression, a cationic lipid, a non-cationic lipid, and a conjugated lipid that inhibits aggregation of particles.

The cationic lipid may be, e.g., N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxypropylamine (DODMA), 1,2-DiLinoleyloxy-N,N-dimethylaminopropane (DLinDMA), 1,2-Dilinolenyloxy-N,N-dimethylaminopropane (DLendMA), or mixtures thereof. The cationic lipid may comprise from about 20 mol % to about 50 mol % or about 40 mol % of the total lipid present in the particle.

The non-cationic lipid may be an anionic lipid or a neutral lipid including, but not limited to, distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylcholine (DPPC), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), cholesterol, or mixtures thereof. The non-cationic lipid may comprise from about 5 mol % to about 90 mol % or about 20 mol % of the total lipid present in the particle.

The conjugated lipid that inhibits aggregation of particles may be a polyethyleneglycol (PEG)-lipid conjugate, a polyamide (ATTA)-lipid conjugate, a cationic-polymer-lipid conjugates (CPLs), or mixtures thereof. In one preferred embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be a PEG-dilauryloxypropyl (C12), a PEG-dimyristyloxypropyl (C14), a PEG-dipalmityloxypropyl (C16), or a PEG-distearyloxypropyl (C18). In some embodiments, the conjugated lipid that inhibits aggregation of particles is a CPL that has the formula: A-W-Y, wherein A is a lipid moiety, W is a hydrophilic polymer, and Y is a polycationic moiety. W may be a polymer selected from the group consisting of PEG, polyamide, polylactic acid, polyglycolic acid, polylactic acid/polyglycolic acid copolymers, or combinations thereof, the polymer having a molecular weight of from about 250 to about 7000 daltons. In some embodiments, Y has at least 4 positive charges at a selected pH. In other embodiments, Y may be lysine, arginine, asparagine, glutamine, derivatives thereof, or combinations thereof. The conjugated lipid that prevents aggregation of particles may be from 0 mol % to about 20 mol % or about 2 mol % of the total lipid present in the particle.

In some embodiments, the nucleic acid-lipid particle further comprises cholesterol at, e.g., about 10 mol % to about 60 mol %, about 30 mol % to about 50 mol %, or about 48 mol % of the total lipid present in the particle.

In certain embodiments, the siRNA in the nucleic acid-lipid particle is not substantially degraded after exposure of the particle to a nuclease at 37° C. for at least 20, 30, 45, or 60 minutes, or after incubation of the particle in serum at 37° C. for at least 30, 45, or 60 minutes.

In some embodiments, the siRNA is fully encapsulated in the nucleic acid-lipid particle. In other embodiments, the siRNA is complexed with the lipid portion of the particle.

The present invention further provides pharmaceutical compositions comprising the nucleic acid-lipid particles described herein and a pharmaceutically acceptable carrier.

In yet another aspect, the siRNA described herein is used in methods for silencing expression of an influenza virus gene such as PA, PB1, PB2, NP, M1, M2, NS1, and/or NS2 from Influenza A, B, or C virus. In particular, it is an object of the present invention to provide in vitro and in vivo methods for treatment of an influenza virus infection in a mammal by downregulating or silencing the transcription and/or translation of a target influenza virus gene of interest. In one embodiment, the present invention provides a method for introducing an siRNA that silences expression (e.g., mRNA and/or protein levels) of an influenza virus gene into a cell by contacting the cell with an siRNA described herein. In another embodiment, the present invention provides a method for in vivo delivery of an siRNA that silences expression of an influenza virus gene by administering to a mammal an siRNA described herein. Administration of the siRNA can be by any route known in the art, such as, e.g., oral, intranasal, intravenous, intraperitoneal, intramuscular, intra-articular, intralesional, intratracheal, subcutaneous, or intradermal.

In these methods, the siRNA that silences influenza virus gene expression is typically formulated with a carrier system, and the carrier system comprising the siRNA is administered to a mammal requiring such treatment. Alternatively, cells are removed from a mammal such as a human, the siRNA is delivered in vitro using a carrier system, and the cells are then administered to the mammal, such as by injection. Examples of carrier systems suitable for use in the present invention include, but are not limited to, nucleic acid-lipid particles, liposomes, micelles, virosomes, nucleic acid complexes (e.g., lipoplexes, polyplexes, etc.), and mixtures thereof. The carrier system may comprise at least one or a cocktail (e.g., at least two, three, four, five, six, seven, eight, nine, ten, or more) of siRNA molecules that silence influenza virus gene expression. In certain embodiments, the carrier system comprises at least one or a cocktail of the sequences set forth in Tables 1-4 and 7-8, such as, e.g., unmodified or modified (such as 2′OMe-modified) NP 97, NP 171, NP 222, NP 383, NP 411, NP 929, NP 1116, NP 1485, PA 392, and/or PA 783.

In some embodiments, the siRNA is in a nucleic acid-lipid particle comprising the siRNA, a cationic lipid, and a non-cationic lipid. Preferably, the siRNA is in a nucleic acid-lipid particle comprising the siRNA, a cationic lipid, a non-cationic lipid, and a conjugated lipid that inhibits aggregation of particles. A therapeutically effective amount of the nucleic acid-lipid particle can be administered to the mammalian subject (e.g., a rodent such as a mouse or a primate such as a human, chimpanzee, or monkey).

In another embodiment, at least about 1%, 2%, 4%, 6%, 8%, or 10% of the total administered dose of the nucleic acid-lipid particles is present in plasma at about 1, 2, 4, 6, 8, 12, 16, 18, or 24 hours after administration. In a further embodiment, more than about 20%, 30%, or 40% or as much as about 60%, 70%, or 80% of the total administered dose of the nucleic acid-lipid particles is present in plasma at about 1, 4, 6, 8, 10, 12, 20, or 24 hours after administration. In one embodiment, the effect of the siRNA (e.g., downregulation of the target influenza virus sequence) at a site proximal or distal to the site of administration is detectable at about 12, 24, 48, 72, or 96 hours, or at about 6, 8, 10, 12, 14, 16, 18, 19, 20, 22, 24, 26, or 28 days after administration of the nucleic acid-lipid particles. In another embodiment, downregulation of expression of the target influenza virus sequence is detectable at about 12, 24, 48, 72, or 96 hours, or at about 6, 8, 10, 12, 14, 16, 18, 19, 20, 22, 24, 26, or 28 days after administration. In certain instances, downregulation of expression of an influenza virus gene sequence is detected by measuring influenza virus mRNA or protein levels in a biological sample from the mammal. In certain other instances, downregulation of expression of an influenza virus gene sequence is detected by measuring influenza virus load in a biological sample from the mammal. In some embodiments, downregulation of expression of an influenza virus gene sequence is detected by monitoring symptoms associated with influenza virus infection in the mammal. In other embodiments, downregulation of expression of an influenza virus gene sequence is detected by measuring survival of the mammal.

In some embodiments, the mammal has been exposed to a second mammal infected with an influenza virus prior to administration of the nucleic acid-lipid particle. In other embodiments, the mammal has been exposed to a fomite contaminated with an influenza virus prior to administration of the nucleic acid-lipid particle. In certain instances, administration of the nucleic acid-lipid particle reduces the amount of influenza hemagglutinin (HA) protein in the mammal by at least about 40%, 45%, 50%, 55%, 60%, 65%, 70%, 75%, 80%, 85%, 90%, or 95% relative to the amount of influenza HA protein in the absence of the particle.

The nucleic acid-lipid particles are suitable for use in intravenous nucleic acid delivery as they are stable in circulation, of a size required for pharmacodynamic behavior resulting in access to extravascular sites, and target cell populations. The present invention also provides pharmaceutically acceptable compositions comprising nucleic acid-lipid particles.

In yet another aspect, the present invention provides a method for modifying an anti-influenza siRNA having immunostimulatory properties, the method comprising: (a) providing an unmodified siRNA sequence capable of silencing expression of an influenza virus gene selected from the group consisting of PA, PB1, PB2, NP, M1, M2, NS1, and NS2; and (b) modifying the unmodified siRNA sequence by substituting at least one nucleotide in the sense or antisense strand with a modified nucleotide, thereby generating a modified siRNA molecule that is less immunostimulatory than the unmodified siRNA sequence and is capable of silencing expression of the influenza virus gene.

The unmodified siRNA sequence typically comprises a double-stranded region of about 15 to about 60 nucleotides in length (e.g., about 15-60, 15-50, 15-40, 15-30, 15-25, or 19-25 nucleotides in length). In some embodiments, the modified nucleotide includes, but is not limited to, 2′OMe nucleotides, 2′F nucleotides, 2′-deoxy nucleotides, 2′OMOE nucleotides, LNA nucleotides, and mixtures thereof. In certain instances, the unmodified siRNA sequence is modified by substituting at least one, two, three, four, five, six, seven, eight, nine, ten, or more of the uridine nucleotides and/or guanosine nucleotides in the sense or antisense strand with modified uridine nucleotides and/or modified guanosine nucleotides, respectively. Preferably, the unmodified siRNA sequence is modified by substituting all of the uridine nucleotides in the sense or antisense strand with modified uridine nucleotides. In other embodiments, an siRNA with selective uridine nucleotide modifications can further comprise at least one, two, three, four, five, six, seven, eight, nine, ten, or more modified nucleotides such as, for example, modified guanosine nucleotides, modified adenosine nucleotides, modified cytosine nucleotides, and mixtures thereof.

In preferred embodiments, the modified nucleotide comprises a 2′OMe nucleotide (e.g., 2′OMe purine and/or pyrimidine nucleotide) such as, for example, a 2′OMe-guanosine nucleotide, 2′OMe-uridine nucleotide, 2′OMe-adenosine nucleotide, 2′OMe-cytosine nucleotide, and mixtures thereof. In certain embodiments, the modified nucleotide is a 2′OMe-uridine nucleotide, 2′OMe-guanosine nucleotide, or mixtures thereof. In other embodiments, the modified nucleotide is not a 2′OMe-cytosine nucleotide.

In certain instances, the unmodified siRNA sequence comprises at least one, two, three, four, five, six, seven, or more 5′-GU-3′ motifs. The 5′-GU-3′ motif can be in the sense strand, the antisense strand, or both strands of the unmodified siRNA sequence. Preferably, at least one nucleotide in the 5′-GU-3′ motif is substituted with a modified nucleotide. As a non-limiting example, both nucleotides in the 5′-GU-3′ motif can be substituted with modified nucleotides.

In some embodiments, the method further comprises: (c) confirming that the modified siRNA molecule is less immunostimulatory by contacting the modified siRNA molecule with a mammalian responder cell under conditions suitable for the mammalian responder cell to produce a detectable immune response. The mammalian responder cell may be from a naïve mammal (i.e., a mammal that has not previously been in contact with the gene product of the siRNA sequence). The mammalian responder cell may be, e.g., a peripheral blood mononuclear cell (PBMC), a macrophage, and the like. The detectable immune response may comprise production of a cytokine or growth factor such as, e.g., TNF-α, IFN-α, IFN-β, IFN-γ, IL-6, IL-12, or a combination thereof.

In a related aspect, the present invention provides a method for identifying and modifying an anti-influenza siRNA having immunostimulatory properties. The method comprises: (a) contacting an unmodified siRNA sequence capable of silencing expression of an influenza virus gene with a mammalian responder cell under conditions suitable for the mammalian responder cell to produce a detectable immune response, wherein the influenza virus gene is selected from the group consisting of PA, PB1, PB2, NP, M1, M2, NS1, and NS2; (b) identifying the unmodified siRNA sequence as an immunostimulatory siRNA molecule by the presence of a detectable immune response in the mammalian responder cell; and (c) modifying the immunostimulatory siRNA molecule by substituting at least one nucleotide with a modified nucleotide, thereby generating a modified siRNA molecule that is less immunostimulatory than the unmodified siRNA sequence.

The unmodified siRNA sequence typically comprises a double-stranded region of about 15 to about 60 nucleotides in length (e.g., about 15-60, 15-50, 15-40, 15-30, 15-25, or 19-25 nucleotides in length). In some embodiments, the modified nucleotide includes, but is not limited to, 2′OMe nucleotides, 2′F nucleotides, 2′-deoxy nucleotides, 2′OMOE nucleotides, LNA nucleotides, and mixtures thereof. In certain instances, the unmodified siRNA sequence is modified by substituting at least one, two, three, four, five, six, seven, eight, nine, ten, or more of the uridine nucleotides and/or guanosine nucleotides in the sense or antisense strand with modified uridine nucleotides and/or modified guanosine nucleotides, respectively. Preferably, the unmodified siRNA sequence is modified by substituting all of the uridine nucleotides in the sense or antisense strand with modified uridine nucleotides. In other embodiments, an siRNA with selective uridine nucleotide modifications can further comprise at least one, two, three, four, five, six, seven, eight, nine, ten, or more modified nucleotides such as, for example, modified guanosine nucleotides, modified adenosine nucleotides, modified cytosine nucleotides, and mixtures thereof.

In preferred embodiments, the modified nucleotide comprises a 2′OMe nucleotide (e.g., 2′OMe purine and/or pyrimidine nucleotide) such as, for example, a 2′OMe-guanosine nucleotide, 2′OMe-uridine nucleotide, 2′OMe-adenosine nucleotide, 2′OMe-cytosine nucleotide, and mixtures thereof. In certain embodiments, the modified nucleotide is a 2′OMe-uridine nucleotide, 2′OMe-guanosine nucleotide, or mixtures thereof. In other embodiments, the modified nucleotide is not a 2′OMe-cytosine nucleotide.

In certain instances, the unmodified siRNA sequence comprises at least one, two, three, four, five, six, seven, or more 5′-GU-3′ motifs. The 5′-GU-3′ motif can be in the sense strand, the antisense strand, or both strands of the unmodified siRNA sequence. Preferably, at least one nucleotide in the 5′-GU-3′ motif is substituted with a modified nucleotide. As a non-limiting example, both nucleotides in the 5′-GU-3′ motif can be substituted with modified nucleotides.

In some embodiments, the mammalian responder cell is a peripheral blood mononuclear cell (PBMC), a macrophage, and the like. In other embodiments, the detectable immune response comprises production of a cytokine or growth factor such as, for example, TNF-α, IFN-α, IFN-β, IFN-γ, IL-6, IL-12, or a combination thereof.

Other features, objects, and advantages of the invention and its preferred embodiments will become apparent from the detailed description, examples, and claims that follow.

### BRIEF DESCRIPTION OF THE DRAWINGS

FIG. 1 illustrates data demonstrating that the optimal ratio of luciferase plasmid to LF2000 in MDCK cells is 1:4. FIG. 1A shows the luciferase activity in relative light units (RLU) per pg protein from MDCK cells transfected with varying ratios of plasmid:LF2000 at 24 hours. FIG. 1B shows the luciferase activity in relative luciferase levels from MDCK cells transfected with varying ratios of plasmid:LF2000 at 24 hours.

FIG. 2 illustrates data demonstrating that NP 1496 siRNA delivered at an siRNA:LF2000 ratio of 1:4 knocks down influenza virus by about 60%. FIG. 2A shows influenza virus infection of MDCK cells at 48 hours after 5 hours of pretreatment with NP1496 siRNA. FIG. 2B shows the percent knockdown of influenza virus in MDCK cells at 48 hours.

FIG. 3 illustrates data demonstrating that NP and PA siRNA display potent anti-influenza activity in an in vitro MDCK cell assay. FIG. 3A shows influenza virus infection of MDCK cells at 48 hours after 5 hours of pretreatment with siRNA. FIG. 3B shows the percentage of HA relative to a virus only control at 48 hours in MDCK cells infected with a 1:800 dilution of influenza virus and transfected with 4 μg/ml siRNA.

FIG. 4 illustrates data demonstrating that NP 411, NP 929, NP 1116, and NP 1496 siRNA comprising selective 2′OMe modifications to the sense strand maintain influenza knockdown activity in vitro in MDCK cells. FIG. 4A shows influenza virus infection of MDCK cells at 48 hours after 5 hours of pretreatment with modified or unmodified siRNA. FIG. 4B shows the percentage of HA relative to a virus only control at 48 hours in MDCK cells infected with a 1:800 dilution of influenza virus and transfected with 2 pg/ml modified or unmodified siRNA.

FIG. 5 illustrates data demonstrating that selective 2′OMe modifications to the sense strand of NP 1496 siRNA do not negatively affect influenza knockdown activity when compared to unmodified counterpart sequences or control sequences.

FIG. 6 illustrates data demonstrating that NP and PA siRNA comprising selective 2′OMe modifications to the sense strand display potent anti-influenza activity in an in vitro MDCK cell assay.

FIG. 7 illustrates data demonstrating that combinations of 2′OMe-modified siRNA provide enhanced influenza knockdown in vitro in MDCK cells. FIG. 7A shows influenza virus infection of MDCK cells at 48 hours after 5 hours of pretreatment with various combinations of modified siRNA. FIG. 7B shows the percentage of HA relative to a virus only control at 48 hours in MDCK cells infected with a 1:800 dilution of influenza virus and transfected with 2 μg/ml modified siRNA.

FIG. 8 illustrates data demonstrating that selective 2′OMe modifications to NP 1496 siRNA abrogates interferon induction in an in vitro cell culture system.

FIG. 9 illustrates data demonstrating that selective 2′OMe modifications to NP 1496 siRNA abrogates the interferon induction associated with systemic administration of the native duplex complexed with the cationic polymer polyethylenimine (PEI).

FIG. 10 illustrates data demonstrating that lipid encapsulated NP 1496 siRNA is capable of viral knockdown in vivo. FIG. 10A shows the HA unit per lung 48 hours after inoculation with influenza virus in mice pretreated with SNALP-encapsulated NP 1496 siRNA. FIG. 10B shows the percentage of HA per lung relative to a PBS control 48 hours after inoculation with influenza virus in mice pretreated with SNALP-encapsulated NP 1496 siRNA.

### DETAILED DESCRIPTION OF THE INVENTION

### I. Introduction

The present invention is based on the discovery that silencing influenza gene expression is an effective means to treat influenza virus (e.g., Influenza A, B, or C virus) infection. Accordingly, the present invention provides siRNA molecules comprising a double-stranded region of about 15 to about 60 nucleotides in length that silence expression of an influenza gene (e.g., PA, PB1, PB2, NP, M1, M2, NS1, and/or NS2). The anti-influenza siRNA molecules of the present invention can be modified or unmodified. Advantageously, the selective incorporation of modifications within the double-stranded region of the siRNA duplex provides siRNA molecules which retain the capability of silencing the expression of a target influenza gene, but are less immunostimulatory than corresponding unmodified siRNA.

The present invention also provides nucleic acid-lipid particles that target influenza gene expression comprising an siRNA that silences influenza gene expression, a cationic lipid, and a non-cationic lipid. In certain instances, the nucleic acid-lipid particles can further comprise a conjugated lipid that inhibits aggregation of particles. The present invention further provides methods of silencing influenza gene expression by administering the siRNA molecules described herein to a mammalian subject. In addition, the present invention provides methods of treating a subject who has been exposed to influenza virus or is exhibiting symptoms of influenza virus infection by administering the siRNA molecules described herein.

### II. Definitions

As used herein, the following terms have the meanings ascribed to them unless specified otherwise.

The terms “influenza virus” or “flu virus” refer to single-stranded RNA viruses belonging to the family Orthomyxoviridae and include, e.g., Influenza A, B, and C viruses, each of which have different nucleoproteins (see, e.g., Steinhauer et al., Annu. Rev. Genet., 36:305-332 (2002); and Neumann et al., J. Gen. Virol., 83:2635-2662 (2002)). The influenza virus genome contains eight separate segments of RNA. One segment encodes nucleoprotein (NP); one segment encodes two matrix proteins (M1 and M2); one segment encodes two nonstructural proteins (NS1 and NS2); three segments each encode one RNA polymerase (PA, PB1, and PB2); one segment encodes neuraminidase (NA); and one segment encodes haemagglutinin (HA). Two distinct neuraminidases, N1 and N2, have been found in human infections and seven neuraminidases have been found in non-human infections. Three distinct hemagglutinins, H1, H2, and H3, have been found in human infections and nine hemaglutinins have been found in non-human infections. Influenza A virus NP sequences are set forth in, e.g., Genbank Accession Nos. AY818138 (SEQ ID NO:1); NC_{—}004522 (SEQ ID NO:2); NC_{—}007360 (SEQ ID NO:3); AB166863; AB188817; AB189046; AB189054; AB189062; AY646169; AY646177; AY651486; AY651493; AY651494; AY651495; AY651496; AY651497; AY651498; AY651499; AY651500; AY651501; AY651502; AY651503; AY651504; AY651505; AY651506; AY651507; AY651509; AY651528; AY770996; AY790308; AY818138; and AY818140. Influenza A virus PA sequences are set forth in, e.g., Genbank Accession Nos. AY818132 (SEQ ID NO:4); AF389117 (SEQ ID NO:5); AY790280; AY646171; AY818132; AY818133; AY646179; AY818134; AY551934; AY651613; AY651610; AY651620; AY651617; AY651600; AY651611; AY651606; AY651618; AY651608; AY651607; AY651605; AY651609; AY651615; AY651616; AY651640; AY651614; AY651612; AY651621; AY651619; AY770995; and AY724786.

The term “interfering RNA” or “RNAi” or “interfering RNA sequence” refers to double-stranded RNA (i.e., duplex RNA) that is capable of silencing, reducing, or inhibiting expression of a target gene (i.e., by mediating the degradation of mRNAs which are complementary to the sequence of the interfering RNA) when the interfering RNA is in the same cell as the target gene. Interfering RNA thus refers to the double-stranded RNA formed by two complementary strands or by a single, self-complementary strand. Interfering RNA may have substantial or complete identity to the target gene or may comprise a region of mismatch (i.e., a mismatch motif). The sequence of the interfering RNA can correspond to the full length target gene, or a subsequence thereof.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 20-24, 21-22, or 21-23 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single-stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99:14236-14240 (2002); Byrom et al., Ambion TechNotes, 10:4-6 (2003); Kawasaki et al., Nucleic Acids Res., 31:981-987 (2003); Knight et al., Science, 293:2269-2271 (2001); and Robertson et al., J. Biol. Chem., 243:82-91 (1968)). Preferably, dsRNA are at least 50 nucleotides to about 100, 200, 300, 400, or 500 nucleotides in length. A dsRNA may be as long as 1000, 1500, 2000, or 5000 nucleotides in length, or longer. The dsRNA can encode for an entire gene transcript or a partial gene transcript.

As used herein, the term “mismatch motif” or “mismatch region” refers to a portion of an siRNA sequence that does not have 100% complementarity to its target sequence. An siRNA may have at least one, two, three, four, five, six, or more mismatch regions. The mismatch regions may be contiguous or may be separated by 1, 2, 3, 4, 5, 6, 7, 8, 9, 10, 11, 12, or more nucleotides. The mismatch motifs or regions may comprise a single nucleotide or may comprise two, three, four, five, or more nucleotides.

The phrase “inhibiting expression of a target gene” refers to the ability of an siRNA molecule of the present invention to silence, reduce, or inhibit expression of a target gene (e.g., an influenza gene). To examine the extent of gene silencing, a test sample (e.g., a biological sample from an organism of interest expressing the target gene or a sample of cells in culture expressing the target gene) is contacted with an siRNA that silences, reduces, or inhibits expression of the target gene. Expression of the target gene in the test sample is compared to expression of the target gene in a control sample that is not contacted with the siRNA. Control samples are assigned a value of 100%. Silencing, inhibition, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, 15%, 10%, 5%, or 0%. Suitable assays include, e.g., examination of protein or mRNA levels using techniques known to those of skill in the art such as dot blots, Northern blots, in situ hybridization, ELISA, immunoprecipitation, enzyme function, as well as phenotypic assays known to those of skill in the art.

The terms “substantially identical” or “substantial identity,” in the context of two or more nucleic acids, refer to two or more sequences or subsequences that are the same or have a specified percentage of nucleotides that are the same (i.e., at least about 60%, preferably at least about 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. Preferably, the substantial identity exists over a region that is at least about 5, 10, 15, 20, 25, 30, 35, 40, 45, 50, 55, or 60 nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of a number of contiguous positions selected from the group consisting of from about 5 to about 60, usually about 10 to about 45, more usually about 15 to about 30, in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math., 2:482 (1981), by the homology alignment algorithm of Needleman and Wunsch, J. Mol. Biol., 48:443 (1970), by the search for similarity method of Pearson and Lipman, Proc. Natl. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. (1995 supplement)).

A preferred example of algorithms that are suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids of the invention. Software for performing BLAST analyses is publicly available through the National Center for Biotechnology Information (http://www.ncbi.nlm.nih.gov/).

The BLAST algorithm also performs a statistical analysis of the similarity between two sequences (see, e.g., Karlin and Altschul, Proc. Natl. Acad. Sci. USA, 90:5873-5787 (1993)). One measure of similarity provided by the BLAST algorithm is the smallest sum probability (P(N)), which provides an indication of the probability by which a match between two nucleotide sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (P1, PAC, BAC, YAC, artificial chromosomes), expression cassettes, chimeric sequences, chromosomal DNA, or derivatives and combinations of these groups. RNA may be in the form of siRNA, mRNA, tRNA, rRNA, tRNA, vRNA, and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see, e.g., Batzer et al., Nucleic Acid Res., 19:5081 (1991); Ohtsuka et al., J. Biol. Chem., 260:2605-2608 (1985); and Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “gene” refers to a nucleic acid (e.g., DNA or RNA) sequence that comprises partial length or entire length coding sequences necessary for the production of a polypeptide or precursor polypeptide (e.g., an influenza polypeptide).

“Gene product,” as used herein, refers to a product of a gene such as an RNA transcript or a polypeptide.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

“Lipid vesicle” refers to any lipid composition that can be used to deliver a compound such as an siRNA including, but not limited to, liposomes, wherein an aqueous volume is encapsulated by an amphipathic lipid bilayer; or wherein the lipids coat an interior comprising a large molecular component, such as a plasmid comprising an interfering RNA sequence, with a reduced aqueous interior; or lipid aggregates or micelles, wherein the encapsulated component is contained within a relatively disordered lipid mixture. The term lipid vesicle encompasses any of a variety of lipid-based carrier systems including, without limitation, SPLPs, pSPLPs, SNALPs, liposomes, micelles, virosomes, lipid-nucleic acid complexes, and mixtures thereof.

As used herein, “lipid encapsulated” can refer to a lipid formulation that provides a compound such as an siRNA with full encapsulation, partial encapsulation, or both. In a preferred embodiment, the nucleic acid is fully encapsulated in the lipid formulation (e.g., to form an SPLP, pSPLP, SNALP, or other nucleic acid-lipid particle).

As used herein, the term “SNALP” refers to a stable nucleic acid-lipid particle, including SPLP. A SNALP represents a vesicle of lipids coating a reduced aqueous interior comprising a nucleic acid (e.g., siRNA, ssDNA, dsDNA, ssRNA, micro RNA (miRNA), short hairpin RNA (shRNA), dsRNA, or a plasmid, including plasmids from which an interfering RNA is transcribed). As used herein, the term “SPLP” refers to a nucleic acid-lipid particle comprising a nucleic acid (e.g., a plasmid) encapsulated within a lipid vesicle. SNALPs and SPLPs typically contain a cationic lipid, a non-cationic lipid, and a lipid that prevents aggregation of the particle (e.g., a PEG-lipid conjugate). SNALPs and SPLPs are extremely useful for systemic applications, as they exhibit extended circulation lifetimes following intravenous (i.v.) injection, accumulate at distal sites (e.g., sites physically separated from the administration site) and can mediate expression of the transfected gene at these distal sites. SPLPs include “pSPLP,” which comprise an encapsulated condensing agent-nucleic acid complex as set forth in PCT Publication No. WO 00/03683.

The nucleic acid-lipid particles of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids, when present in the nucleic acid-lipid particles of the present invention, are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; and 6,815,432; and PCT Publication No. WO 96/40964.

The term “vesicle-forming lipid” is intended to include any amphipathic lipid having a hydrophobic moiety and a polar head group, and which by itself can form spontaneously into bilayer vesicles in water, as exemplified by most phospholipids.

The term “vesicle-adopting lipid” is intended to include any amphipathic lipid that is stably incorporated into lipid bilayers in combination with other amphipathic lipids, with its hydrophobic moiety in contact with the interior, hydrophobic region of the bilayer membrane, and its polar head group moiety oriented toward the exterior, polar surface of the membrane. Vesicle-adopting lipids include lipids that on their own tend to adopt a nonlamellar phase, yet which are capable of assuming a bilayer structure in the presence of a bilayer-stabilizing component. A typical example is dioleoylphosphatidylethanolamine (DOPE). Bilayer stabilizing components include, but are not limited to, conjugated lipids that inhibit aggregation of nucleic acid-lipid particles, polyamide oligomers (e.g., ATTA-lipid derivatives), peptides, proteins, detergents, lipid-derivatives, PEG-lipid derivatives such as PEG coupled to dialkyloxypropyls, PEG coupled to diacylglycerols, PEG coupled to phosphatidyl-ethanolamines, PEG conjugated to ceramides (see, e.g., U.S. Pat. No. 5,885,613), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties.

The term “amphipathic lipid” refers, in part, to any suitable material wherein the hydrophobic portion of the lipid material orients into a hydrophobic phase, while the hydrophilic portion orients toward the aqueous phase. Amphipathic lipids are usually the major component of a lipid vesicle. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, pahnitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipid described above can be mixed with other lipids including triglycerides and sterols.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any neutral lipid as described above as well as anionic lipids.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH (e.g., pH of about 7.0). It has been surprisingly found that cationic lipids comprising alkyl chains with multiple sites of unsaturation, e.g., at least two or three sites of unsaturation, are particularly useful for forming nucleic acid-lipid particles with increased membrane fluidity. A number of cationic lipids and related analogs, which are also useful in the present invention, have been described in U.S. Patent Publication No. 20060083780; U.S. Pat. Nos. 5,208,036; 5,264,618; 5,279,833; 5,283,185; 5,753,613; and 5,785,992; and PCT Publication No. WO 96/10390. Examples of cationic lipids include, but are not limited to, N,N-dioleyl-N,N-dimethylammonium chloride (DODAC), dioctadecyldimethylammonium (DODMA), distearyldimethylammonium (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 1,2-dilinoleyloxy-N,N-dimethylaminopropane (DLinDMA), 1,2-dilinolenyloxy-N,N-dimethylaminopropane (DLenDMA), and mixtures thereof. As a non-limiting example, cationic lipids that have a positive charge below physiological pH include, but are not limited to, DODAP, DODMA, and DSDMA. In some cases, the cationic lipids comprise a protonatable tertiary amine head group, C18 alkyl chains, ether linkages between the head group and alkyl chains, and 0 to 3 double bonds. Such lipids include, e.g., DSDMA, DLinDMA, DLenDMA, and DODMA. The cationic lipids may also comprise ether linkages and pH titratable head groups. Such lipids include, e.g., DODMA.

The term “hydrophobic lipid” refers to compounds having apolar groups that include, but are not limited to, long chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N-N-dialkylamino, 1,2-diacyloxy-3-aminopropane, and 1,2-dialkyl-3-aminopropane.

The term “fusogenic” refers to the ability of a liposome, a SNALP, or other drug delivery system to fuse with membranes of a cell. The membranes can be either the plasma membrane or membranes surrounding organelles, e.g., endosome, nucleus, etc.

As used herein, the term “aqueous solution” refers to a composition comprising in whole, or in part, water.

As used herein, the term “organic lipid solution” refers to a composition comprising in whole, or in part, an organic solvent having a lipid.

“Distal site,” as used herein, refers to a physically separated site, which is not limited to an adjacent capillary bed, but includes sites broadly distributed throughout an organism.

“Serum-stable” in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

“Systemic delivery,” as used herein, refers to delivery that leads to a broad biodistribution of a compound such as an siRNA within an organism. Some techniques of administration can lead to the systemic delivery of certain compounds, but not others. Systemic delivery means that a useful, preferably therapeutic, amount of a compound is exposed to most parts of the body. To obtain broad biodistribution generally requires a blood lifetime such that the compound is not rapidly degraded or cleared (such as by first pass organs (liver, lung, etc.) or by rapid, nonspecific cell binding) before reaching a disease site distal to the site of administration. Systemic delivery of nucleic acid-lipid particles can be by any means known in the art including, for example, intravenous, subcutaneous, and intraperitoneal. In a preferred embodiment, systemic delivery of nucleic acid-lipid particles is by intravenous delivery.

“Local delivery,” as used herein, refers to delivery of a compound such as an siRNA directly to a target site within an organism. For example, a compound can be locally delivered by direct injection into a disease site such as a tumor or other target site such as a site of inflammation or a target organ such as the liver, heart, pancreas, kidney, and the like.

The term “mammal” refers to any mammalian species such as a human, mouse, rat, dog, cat, hamster, guinea pig, rabbit, livestock, and the like.

“Fomite” as used herein refers to any inanimate object that when contaminated with a viable pathogen (e.g., an influenza virus) can transfer the pathogen to a host. Typical fomites include, e.g., hospital and clinic waiting and examination room surfaces (e.g., floors, walls, ceilings, curtains, carpets), needles, syringes, scalpels, catheters, brushes, stethoscopes, laryngoscopes, thermometers, tables, bedding, towels, eating utensils, and the like.

### III. siRNAs

The present invention provides an interfering RNA that silences (e.g., partially or completely inhibits) expression of a gene of interest (i.e., an influenza gene). An interfering RNA can be provided in several forms. For example, an interfering RNA can be provided as one or more isolated small-interfering RNA (siRNA) duplexes, longer double-stranded RNA (dsRNA), or as siRNA or dsRNA transcribed from a transcriptional cassette in a DNA plasmid. The interfering RNA may also be chemically synthesized. The interfering RNA can be administered alone or co-administered (i.e., concurrently or consecutively) with conventional agents used to treat an influenza virus infection.

In one aspect, the interfering RNA is an siRNA molecule that is capable of silencing expression of a target sequence (e.g., PA, PB1, PB2, NP, M1, M2, NS1, or NS2) from an influenza virus. Suitable siRNA sequences are set forth in, e.g., Tables 1-4 and 7-8. Particularly preferred siRNA sequences are set forth in Tables 7-8. For any of the sequences set forth in Tables 1-4 and 7-8, thymine (i.e., “T”) can substituted with uracil (i.e., “U”) and uracil can be substituted with thymine. In some embodiments, the siRNA molecules are about 15 to 60 nucleotides in length. The synthesized or transcribed siRNA can have 3′ overhangs of about 1-4 nucleotides, preferably of about 2-3 nucleotides, and 5′ phosphate termini. In some embodiments, the siRNA lacks terminal phosphates.

In certain embodiments, the siRNA molecules of the present invention are chemically modified as described in, e.g., U.S. patent application Ser. No. ______, filed Nov. 2, 2006 (Attorney Docket No. 020801-005020US), the teachings of which are herein incorporated by reference in their entirety for all purposes. The modified siRNA molecules are capable of silencing expression of a target sequence (e.g., PA, PB1, PB2, NP, M1, M2, NS1, or NS2) from an influenza virus, are about 15 to 60 nucleotides in length, are less immunostimulatory than a corresponding unmodified siRNA sequence, and retain RNAi activity against the target sequence. In some embodiments, the modified siRNA contains at least one 2′-O-methyl (2′OMe) purine or pyrimidine nucleotide such as a 2′OMe-guanosine, 2′OMe-uridine, 2′OMe-adenosine, and/or 2′OMe-cytosine nucleotide. In preferred embodiments, one or more of the uridine and/or guanosine nucleotides are modified. The modified nucleotides can be present in one strand (i.e., sense or antisense) or both strands of the siRNA. Preferably, modified siRNA molecules are chemically synthesized. The modified siRNA can have 3′ overhangs of about 1-4 nucleotides, preferably of about 2-3 nucleotides, and 5′ phosphate termini. In some embodiments, the modified siRNA lacks terminal phosphates. In other embodiments, the modified siRNA lacks overhangs (i.e., has blunt ends).

The modified siRNA generally comprises from about 1% to about 100% (e.g., about 1%, 5%, 10%, 15%, 20%, 25%, 30%, 35%, 40%, 45%, 50%, 55%, 60%, 65%, 70%, 75%, 80%, 85%, 90%, 95%, or 100%) modified nucleotides in the double-stranded region of the siRNA duplex. In one preferred embodiment, less than about 20% (e.g., less than about 20%, 19%, 18%, 17%, 16%, 15%, 14%, 13%, 12%, 11%, 10%, 9%, 8%, 7%, 6%, 5%, 4%, 3%, 2%, or 1%) or from about 1% to about 20% (e.g., from about 1%-20%, 2%-20%, 3%-20%, 4%-20%,5%-20%, 6%-20%, 7%-20%, 8%-20%, 9%-20%, 10%-20%, 11%-20%, 12%-20%, 13%-20%, 14%-20%, 15%-20%, 16%-20%, 17%-20%, 18%-20%, or 19%-20%) of the nucleotides in the double-stranded region comprise modified nucleotides. In another preferred embodiment, e.g., when one or both strands of the siRNA are selectively modified at uridine and/or guanosine nucleotides, the resulting modified siRNA can comprise less than about 30% modified nucleotides (e.g., less than about 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% modified nucleotides) or from about 1% to about 30% modified nucleotides (e.g., from about 1%-30%, 2%-30%, 3%-30%, 4%-30%, 5%-30%, 6%-30%, 7%-30%, 8%-30%, 9%-30%, 10%-30%, 11%-30%, 12%-30%, 13%-30%, 14%-30%, 15%-30%, 16%-30%, 17%-30%, 18%-30%, 19%-30%, 20%-30%, 21%-30%, 22%-30%, 23%-30%, 24%-30%, 25%-30%, 26%-30%, 27%-30%, 28%-30%, or 29%-30% modified nucleotides).

In some embodiments, the siRNA molecules described herein comprise at least one region of mismatch with its target sequence. As used herein, the term “region of mismatch” refers to a region of an siRNA that does not have 100% complementarity to its target sequence. An siRNA may have at least one, two, or three regions of mismatch. The regions of mismatch may be contiguous or may be separated by one or more nucleotides. The regions of mismatch may comprise a single nucleotide or may comprise two, three, four, or more nucleotides.

A. Selection of siRNA Sequences

Suitable siRNA sequences can be identified using any means known in the art. Typically, the methods described in Elbashir et al., Nature, 411:494-498 (2001) and Elbashir et al., EMBO J., 20:6877-6888 (2001) are combined with rational design rules set forth in Reynolds et al., Nature Biotech., 22:326-330 (2004).

Generally, the nucleotide sequence 3′ of the AUG start codon of a transcript from the target gene of interest is scanned for dinucleotide sequences (e.g., AA, NA, CC, GG, or UU, wherein N=C, G, or U) (see, e.g., Elbashir et al., EMBO J., 20:6877-6888 (2001)). The nucleotides immediately 3′ to the dinucleotide sequences are identified as potential siRNA target sequences. Typically, the 19, 21, 23, 25, 27, 29, 31, 33, 35, or more nucleotides immediately 3′ to the dinucleotide sequences are identified as potential siRNA target sites. In some embodiments, the dinucleotide sequence is an AA or NA sequence and the 19 nucleotides immediately 3′ to the AA or NA dinucleotide are identified as a potential siRNA target site. siRNA target sites are usually spaced at different positions along the length of the target gene. To further enhance silencing efficiency of the siRNA sequences, potential siRNA target sites may be analyzed to identify sites that do not contain regions of homology to other coding sequences, e.g., in the target cell or organism. For example, a suitable siRNA target site of about 21 base pairs typically will not have more than 16-17 contiguous base pairs of homology to coding sequences in the target cell or organism. If the siRNA sequences are to be expressed from an RNA Pol III promoter, siRNA target sequences lacking more than 4 contiguous A's or T's are selected.

Once a potential siRNA sequence has been identified, the sequence can be analyzed using a variety of criteria known in the art. For example, to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. siRNA design tools that incorporate algorithms that assign suitable values of each of these features and are useful for selection of siRNA can be found at, e.g., http://boz094.ust.hk/RNAi/siRNA. One of skill in the art will appreciate that sequences with one or more of the foregoing characteristics may be selected for further analysis and testing as potential siRNA sequences.

Additionally, potential siRNA target sequences with one or more of the following criteria can often be eliminated as siRNA: (1) sequences comprising a stretch of 4 or more of the same base in a row; (2) sequences comprising homopolymers of Gs (i.e., to reduce possible non-specific effects due to structural characteristics of these polymers; (3) sequences comprising triple base motifs (e.g., GGG, CCC, AAA, or TTT); (4) sequences comprising stretches of 7 or more G/Cs in a row; and (5) sequences comprising direct repeats of 4 or more bases within the candidates resulting in internal fold-back structures. However, one of skill in the art will appreciate that sequences with one or more of the foregoing characteristics may still be selected for further analysis and testing as potential siRNA sequences.

In some embodiments, potential siRNA target sequences may be further analyzed based on siRNA duplex asymmetry as described in, e.g., Khvorova et al., Cell, 115:209-216 (2003); and Schwarz et al., Cell, 115:199-208 (2003). In other embodiments, potential siRNA target sequences may be further analyzed based on secondary structure at the mRNA target site as described in, e.g., Luo et al., Biophys. Res. Commun., 318:303-310 (2004). For example, mRNA secondary structure can be modeled using the Mfold algorithm (available at http://www.bioinfo.rpi.edu/applications/mfold/ma/form1.cgi) to select siRNA sequences which favor accessibility at the mRNA target site where less secondary structure in the form of base-pairing and stem-loops is present.

Once a potential siRNA sequence has been identified, the sequence can be analyzed for the presence of any immunostimulatory properties, e.g., using an in vitro cytokine assay or an in vivo animal model. Motifs in the sense and/or antisense strand of the siRNA sequence such as GU-rich motifs (e.g., 5′-GU-3′, 5′-UGU-3′, 5′-GUGU-3′, 5′-UGUGU-3′, etc.) can also provide an indication of whether the sequence may be immunostimulatory. Once an siRNA molecule is found to be immunostimulatory, it can then be modified to decrease its immunostimulatory properties as described herein. As a non-limiting example, an siRNA sequence can be contacted with a mammalian responder cell under conditions such that the cell produces a detectable immune response to determine whether the siRNA is an immunostimulatory or a non-immunostimulatory siRNA. The mammalian responder cell may be from a naïve mammal (i.e., a mammal that has not previously been in contact with the gene product of the siRNA sequence). The mammalian responder cell may be, e.g., a peripheral blood mononuclear cell (PBMC), a macrophage, and the like. The detectable immune response may comprise production of a cytokine or growth factor such as, e.g., TNF-α, IFN-α, IFN-β, IFN-γ, IL-6, IL-12, or a combination thereof. An siRNA molecule identified as being immunostimulatory can then be modified to decrease its immunostimulatory properties by replacing at least one of the nucleotides on the sense and/or antisense strand with modified nucleotides. For example, less than about 30% (e.g., less than about 30%, 25%, 20%, 15%, 10%, or 5%) of the nucleotides in the double-stranded region of the siRNA duplex can be replaced with modified nucleotides such as 2′OMe nucleotides. The modified siRNA can then be contacted with a mammalian responder cell as described above to confirm that its immunostimulatory properties have been reduced or abrogated.

Suitable in vitro assays for detecting an immune response include, but are not limited to, the double monoclonal antibody sandwich immunoassay technique of David et al. (U.S. Pat. No. 4,376,110); monoclonal-polyclonal antibody sandwich assays (Wide et al., in Kirkham and Hunter, eds., Radioimmunoassay Methods, E. and S. Livingstone, Edinburgh (1970)); the “Western blot” method of Gordon et al. (U.S. Pat. No. 4,452,901); immunoprecipitation of labeled ligand (Brown et al., J. Biol. Chem., 255:4980-4983 (1980)); enzyme-linked immunosorbent assays (ELISA) as described, for example, by Raines et al., J. Biol. Chem., 257:5154-5160 (1982); immunocytochemical techniques, including the use of fluorochromes (Brooks et al., Clin. Exp. Immunol., 39:477 (1980)); and neutralization of activity (Bowen-Pope et al., Proc. Natl. Acad. Sci. USA, 81:2396-2400 (1984)). In addition to the immunoassays described above, a number of other immunoassays are available, including those described in U.S. Pat. Nos. 3,817,827; 3,850,752; 3,901,654; 3,935,074; 3,984,533; 3,996,345; 4,034,074; and 4,098,876.

A non-limiting example of an in vivo model for detecting an immune response includes an in vivo mouse cytokine induction assay that can be performed as follows: (1) siRNA can be administered by standard intravenous injection in the lateral tail vein; (2) blood can be collected by cardiac puncture about 6 hours after administration and processed as plasma for cytokine analysis; and (3) cytokines can be quantified using sandwich ELISA kits according to the manufacturer's instructions (e.g., mouse and human IFN-α (PBL Biomedical; Piscataway, N.J.); human IL-6 and TNF-α (eBioscience; San Diego, Calif.); and mouse IL-6, TNF-α, and IFN-γ (BD Biosciences; San Diego, Calif.)).

Monoclonal antibodies that specifically bind cytokines and growth factors are commercially available from multiple sources and can be generated using methods known in the art (see, e.g., Kohler and Milstein, Nature, 256: 495-497 (1975); and Harlow and Lane, ANTIBODIES, A LABORATORY MANUAL, Cold Spring Harbor Publication, New York (1999)). Generation of monoclonal antibodies has been previously described and can be accomplished by any means known in the art (see, e.g., Buhring et al. in Hybridoma, Vol. 10, No. 1, pp. 77-78 (1991)). In some methods, the monoclonal antibody is labeled (e.g., with any composition detectable by spectroscopic, photochemical, biochemical, electrical, optical, chemical means, and the like) to facilitate detection.

B. Generating siRNA Molecules

siRNA molecules can be provided in several forms including, e.g., as one or more isolated small-interfering RNA (siRNA) duplexes, as longer double-stranded RNA (dsRNA), or as siRNA or dsRNA transcribed from a transcriptional cassette in a DNA plasmid. The siRNA sequences may have overhangs (e.g., 3′ or 5′ overhangs as described in Elbashir et al., Genes Dev., 15:188 (2001) or Nykänen et al., Cell, 107:309 (2001)), or may lack overhangs (i.e., have blunt ends).

An RNA population can be used to provide long precursor RNAs, or long precursor RNAs that have substantial or complete identity to a selected target sequence can be used to make the siRNA. The RNAs can be isolated from cells or tissue, synthesized, and/or cloned according to methods well known to those of skill in the art. The RNA can be a mixed population (obtained from cells or tissue, transcribed from cDNA, subtracted, selected, etc.), or can represent a single target sequence. RNA can be naturally occurring (e.g., isolated from tissue or cell samples), synthesized in vitro (e.g., using T7 or SP6 polymerase and PCR products or a cloned cDNA), or chemically synthesized.

To form a long dsRNA, for synthetic RNAs, the complement is also transcribed in vitro and hybridized to form a dsRNA. If a naturally occurring RNA population is used, the RNA complements are also provided (e.g., to form dsRNA for digestion by E. coli RNAse III or Dicer), e.g., by transcribing cDNAs corresponding to the RNA population, or by using RNA polymerases. The precursor RNAs are then hybridized to form double stranded RNAs for digestion. The dsRNAs can be directly administered to a subject or can be digested in vitro prior to administration.

Alternatively, one or more DNA plasmids encoding one or more siRNA templates are used to provide siRNA. siRNA can be transcribed as sequences that automatically fold into duplexes with hairpin loops from DNA templates in plasmids having RNA polymerase III transcriptional units, for example, based on the naturally occurring transcription units for small nuclear RNA U6 or human RNase P RNA H1 (see, Brummelkamp et al., Science, 296:550 (2002); Donzé et al., Nucleic Acids Res., 30:e46 (2002); Paddison et al., Genes Dev., 16:948 (2002); Yu et al., Proc. Natl. Acad. Sci. USA, 99:6047 (2002); Lee et al., Nat. Biotech., 20:500 (2002); Miyagishi et al., Nat. Biotech., 20:497 (2002); Paul et al., Nat. Biotech., 20:505 (2002); and Sui et al., Proc. Natl. Acad. Sci. USA, 99:5515 (2002)). Typically, a transcriptional unit or cassette will contain an RNA transcript promoter sequence, such as an H1-RNA or a U6 promoter, operably linked to a template for transcription of a desired siRNA sequence and a termination sequence, comprised of 2-3 uridine residues and a polythymidine (T5) sequence (polyadenylation signal) (Brummelkamp et al., supra). The selected promoter can provide for constitutive or inducible transcription. Compositions and methods for DNA-directed transcription of RNA interference molecules is described in detail in U.S. Pat. No. 6,573,099. The transcriptional unit is incorporated into a plasmid or DNA vector from which the interfering RNA is transcribed. Plasmids suitable for in vivo delivery of genetic material for therapeutic purposes are described in detail in U.S. Pat. Nos. 5,962,428 and 5,910,488. The selected plasmid can provide for transient or stable delivery of a target cell. It will be apparent to those of skill in the art that plasmids originally designed to express desired gene sequences can be modified to contain a transcriptional unit cassette for transcription of siRNA.

Methods for isolating RNA, synthesizing RNA, hybridizing nucleic acids, making and screening cDNA libraries, and performing PCR are well known in the art (see, e.g., Gubler and Hoffman, Gene 25:263-269 (1983); Sambrook et al., supra; Ausubel et al., supra), as are PCR methods (see, U.S. Pat. Nos. 4,683,195 and 4,683,202; PCR Protocols: A Guide to Methods and Applications (Innis et al., eds, 1990)). Expression libraries are also well known to those of skill in the art. Additional basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994).

Preferably, siRNA molecules are chemically synthesized. The single-stranded molecules that comprise the siRNA molecule can be synthesized using any of a variety of techniques known in the art, such as those described in Usman et al., J. Am. Chem. Soc., 109:7845 (1987); Scaringe et al., Nucl. Acids Res., 18:5433 (1990); Wincott et al., Nucl. Acids Res., 23:2677-2684 (1995); and Wincott et al., Methods Mol. Bio., 74:59 (1997). The synthesis of the single-stranded molecules makes use of common nucleic acid protecting and coupling groups, such as dimethoxytrityl at the 5′-end and phosphoramidites at the 3′-end. As a non-limiting example, small scale syntheses can be conducted on an Applied Biosystems synthesizer using a 0.2 μmol scale protocol with a 2.5 min coupling step for 2′-O-methylated nucleotides. Alternatively, syntheses at the 0.2 μmol scale can be performed on a 96-well plate synthesizer from Protogene (Palo Alto, Calif.). However, a larger or smaller scale of synthesis is also within the scope of the present invention. Suitable reagents for synthesis of the siRNA single-stranded molecules, methods for RNA deprotection, and methods for RNA purification are known to those of skill in the art.

The siRNA molecules can also be synthesized via a tandem synthesis technique, wherein both strands are synthesized as a single continuous fragment or strand separated by a cleavable linker that is subsequently cleaved to provide separate fragments or strands that hybridize to form the siRNA duplex. The linker can be a polynucleotide linker or a non-nucleotide linker. The tandem synthesis of siRNA can be readily adapted to both multiwell/multiplate synthesis platforms as well as large scale synthesis platforms employing batch reactors, synthesis columns, and the like. Alternatively, the siRNA molecules can be assembled from two distinct single-stranded molecules, wherein one strand comprises the sense strand and the other comprises the antisense strand of the siRNA. For example, each strand can be synthesized separately and joined together by hybridization or ligation following synthesis and/or deprotection. In certain other instances, the siRNA molecules can be synthesized as a single continuous fragment, where the self-complementary sense and antisense regions hybridize to form an siRNA duplex having hairpin secondary structure.

C. Modifying siRNA Sequences

In certain aspects, the siRNA molecules of the present invention comprise a duplex having two strands and at least one modified nucleotide in the double-stranded region, wherein each strand is about 15 to about 60 nucleotides in length. Advantageously, the modified siRNA is less immunostimulatory than a corresponding unmodified siRNA sequence, but retains the capability of silencing the expression of a target sequence.

Examples of modified nucleotides suitable for use in the present invention include, but are not limited to, ribonucleotides having a 2′-O-methyl (2′OMe), 2′-deoxy-2′-fluoro (2′F), 2′-deoxy, 5-C-methyl, 2′-O-(2-methoxyethyl) (MOE), 4′-thio, 2′-amino, or 2′-C-allyl group. Modified nucleotides having a Northern conformation such as those described in, e.g., Saenger, Principles of Nucleic Acid Structure, Springer-Verlag Ed. (1984), are also suitable for use in the siRNA molecules of the present invention. Such modified nucleotides include, without limitation, locked nucleic acid (LNA) nucleotides (e.g., 2′-O, 4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′F) nucleotides, 2′-deoxy-2′-chloro (2Cl) nucleotides, and 2′-azido nucleotides. In certain instances, the siRNA molecules of the present invention include one or more G-clamp nucleotides. A G-clamp nucleotide refers to a modified cytosine analog wherein the modifications confer the ability to hydrogen bond both Watson-Crick and Hoogsteen faces of a complementary guanine nucleotide within a duplex (see, e.g., Lin et al., J. Am. Chem. Soc., 120:8531-8532 (1998)). In addition, nucleotides having a nucleotide base analog such as, for example, C-phenyl, C-naphthyl, other aromatic derivatives, inosine, azole carboxamides, and nitroazole derivatives such as 3-nitropyrrole, 4-nitroindole, 5-nitroindole, and 6-nitroindole (see, e.g., Loakes, Nucl. Acids Res., 29:2437-2447 (2001)) can be incorporated into the siRNA molecules of the present invention.

In certain embodiments, the siRNA molecules of the present invention further comprise one or more chemical modifications such as terminal cap moieties, phosphate backbone modifications, and the like. Examples of terminal cap moieties include, without limitation, inverted deoxy abasic residues, glyceryl modifications, 4′,5′-methylene nucleotides, 1-(β-D-erythrofuranosyl) nucleotides, 4′-thio nucleotides, carbocyclic nucleotides, 1,5-anhydrohexitol nucleotides, L-nucleotides, α-nucleotides, modified base nucleotides, threo-pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3-aminopropyl phosphate, 6-aminohexyl phosphate, 1,2-aminododecyl phosphate, hydroxypropyl phosphate, 1,4-butanediol phosphate, 3′-phosphoramidate, 5′-phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties (see, e.g., U.S. Pat. No. 5,998,203; Beaucage et al., Tetrahedron 49:1925 (1993)). Non-limiting examples of phosphate backbone modifications (i.e., resulting in modified internucleotide linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions (see, e.g., Hunziker et al., Nucleic Acid Analogues: Synthesis and Properties, in Modern Synthetic Methods, VCH, 331-417 (1995); Mesmaeker et al., Novel Backbone Replacements for Oligonucleotides, in Carbohydrate Modifications in Antisense Research, ACS, 24-39 (1994)). Such chemical modifications can occur at the 5′-end and/or 3′-end of the sense strand, antisense strand, or both strands of the siRNA.

In some embodiments, the sense and/or antisense strand can further comprise a 3′-terminal overhang having about 1 to about 4 (e.g., 1, 2, 3, or 4) 2′-deoxy ribonucleotides and/or any combination of modified and unmodified nucleotides. Additional examples of modified nucleotides and types of chemical modifications that can be introduced into the modified siRNA molecules of the present invention are described, e.g., in UK Patent No. GB 2,397,818 B and U.S. Patent Publication Nos. 20040192626 and 20050282188.

The siRNA molecules of the present invention can optionally comprise one or more non-nucleotides in one or both strands of the siRNA. As used herein, the term “non-nucleotide” refers to any group or compound that can be incorporated into a nucleic acid chain in the place of one or more nucleotide units, including sugar and/or phosphate substitutions, and allows the remaining bases to exhibit their activity. The group or compound is abasic in that it does not contain a commonly recognized nucleotide base such as adenosine, guanine, cytosine, uracil, or thymine and therefore lacks a base at the 1′-position.

In other embodiments, chemical modification of the siRNA comprises attaching a conjugate to the siRNA molecule. The conjugate can be attached at the 5′- and/or 3′-end of the sense and/or antisense strand of the siRNA via a covalent attachment such as, e.g., a biodegradable linker. The conjugate can also be attached to the siRNA, e.g., through a carbamate group or other linking group (see, e.g., U.S. Patent Publication Nos. 20050074771, 20050043219, and 20050158727). In certain instances, the conjugate is a molecule that facilitates the delivery of the siRNA into a cell. Examples of conjugate molecules suitable for attachment to the siRNA of the present invention include, without limitation, steroids such as cholesterol, glycols such as polyethylene glycol (PEG), human serum albumin (HSA), fatty acids, carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, and combinations thereof (see, e.g., U.S. Patent Publication Nos. 20030130186, 20040110296, and 20040249178; U.S. Pat. No. 6,753,423). Other examples include the lipophilic moiety, vitamin, polymer, peptide, protein, nucleic acid, small molecule, oligosaccharide, carbohydrate cluster, intercalator, minor groove binder, cleaving agent, and cross-linking agent conjugate molecules described in U.S. Patent Publication Nos. 20050119470 and 20050107325. Yet other examples include the 2′-O-alkyl amine, 2′-O-alkoxyalkyl amine, polyamine, C5-cationic modified pyrimidine, cationic peptide, guanidinium group, amidininium group, cationic amino acid conjugate molecules described in U.S. Patent Publication No. 20050153337. Additional examples include the hydrophobic group, membrane active compound, cell penetrating compound, cell targeting signal, interaction modifier, and steric stabilizer conjugate molecules described in U.S. Patent Publication No. 20040167090. Further examples include the conjugate molecules described in U.S. Patent Publication No. 20050239739. The type of conjugate used and the extent of conjugation to the siRNA molecule can be evaluated for improved pharmacokinetic profiles, bioavailability, and/or stability of the siRNA while retaining RNAi activity. As such, one skilled in the art can screen siRNA molecules having various conjugates attached thereto to identify ones having improved properties and RNAi activity using any of a variety of well-known in vitro cell culture or in vivo animal models.

### IV. Carrier Systems Containing siRNA

In one aspect, the present invention provides carrier systems containing the siRNA molecules described herein. In some embodiments, the carrier system is a lipid-based carrier system such as a stabilized nucleic acid-lipid particle (e.g., SNALP or SPLP), cationic lipid or liposome nucleic acid complexes (i.e., lipoplexes), a liposome, a micelle, a virosome, or a mixture thereof. In other embodiments, the carrier system is a polymer-based carrier system such as a cationic polymer-nucleic acid complex (i.e., polyplex). In additional embodiments, the carrier system is a cyclodextrin-based carrier system such as a cyclodextrin polymer-nucleic acid complex. In further embodiments, the carrier system is a protein-based carrier system such as a cationic peptide-nucleic acid complex. Preferably, the carrier system is a stabilized nucleic acid-lipid particle such as a SNALP or SPLP. One skilled in the art will appreciate that the siRNA molecules of the present invention can also be delivered as naked siRNA.

A. Stabilized Nucleic Acid-Lipid Particles

The stabilized nucleic acid-lipid particles (SNALPs) of the present invention typically comprise an siRNA molecule that targets expression of an influenza virus gene, a cationic lipid (e.g., a cationic lipid of Formula I or II), and a non-cationic lipid. The SNALPs can further comprise a bilayer stabilizing component (i.e., a conjugated lipid that inhibits aggregation of the particles). The SNALPs may comprise at least 1, 2, 3, 4, 5, 6, 7, 8, 9, 10, or more of the siRNA molecules described herein.

The SNALPs of the present invention typically have a mean diameter of about 50 nm to about 150 nm, more typically about 60 nm to about 130 nm, more typically about 70 nm to about 110 nm, most typically about 70 to about 90 nm, and are substantially nontoxic. In addition, the nucleic acids are resistant in aqueous solution to degradation with a nuclease when present in the nucleic acid-lipid particles. Nucleic acid-lipid particles and their method of preparation are disclosed in, e.g., U.S. Pat. Nos. 5,753,613; 5,785,992; 5,705,385; 5,976,567; 5,981,501; 6,110,745; and 6,320,017; and PCT Publication No. WO 96/40964.

1. Cationic Lipids

Any of a variety of cationic lipids may be used in the stabilized nucleic acid-lipid particles of the present invention, either alone or in combination with one or more other cationic lipid species or non-cationic lipid species.

Cationic lipids which are useful in the present invention can be any of a number of lipid species which carry a net positive charge at physiological pH. Such lipids include, but are not limited to, DODAC, DODMA, DSDMA, DOTMA, DDAB, DOTAP, DOSPA, DOGS, DC-Chol, DMRIE, and mixtures thereof. A number of these lipids and related analogs have been described in U.S. Patent Publication No. 20060083780; U.S. Pat. Nos. 5,208,036; 5,264,618; 5,279,833; 5,283,185; and 5,753,613; and 5,785,992; and PCT Publication No. WO 96/10390. Additionally, a number of commercial preparations of cationic lipids are available and can be used in the present invention. These include, for example, LIPOFECTIN® (commercially available cationic liposomes comprising DOTMA and DOPE, from GEBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

Furthermore, cationic lipids of Formula I having the following structures are useful in the present invention. 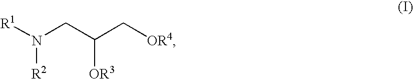
wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C18), etc. In certain other instances, R³ and R⁴ are different, i.e., R³ is tetradectrienyl (C14) and R⁴ is linoleyl (C18). In a preferred embodiment, the cationic lipid of Formula I is symmetrical, i.e., R³ and R⁴ are both the same. In another preferred embodiment, both R³ and R⁴ comprise at least two sites of unsaturation. In some embodiments, R³ and R⁴ are independently selected from dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl. In a particularly preferred embodiments, the cationic lipid of Formula I is DLinDMA or DLenDMA.

Moreover, cationic lipids of Formula II having the following structures are useful in the present invention. 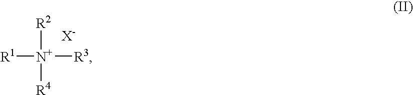
wherein R¹ and R² are independently selected and are H or C₁-C₃ alkyls, R³ and R⁴ are independently selected and are alkyl groups having from about 10 to about 20 carbon atoms, and at least one of R³ and R⁴ comprises at least two sites of unsaturation. In certain instances, R³ and R⁴ are both the same, i.e., R³ and R⁴ are both linoleyl (C18), etc. In certain other instances, R³ and R⁴ are different, i.e., R³ is tetradectrienyl (C14) and R⁴ is linoleyl (C18). In a preferred embodiment, the cationic lipids of the present invention are symmetrical, i.e., R³ and R⁴ are both the same. In another preferred embodiment, both R³ and R⁴ comprise at least two sites of unsaturation. In some embodiments, R³ and R⁴ are independently selected from dodecadienyl, tetradecadienyl, hexadecadienyl, linoleyl, and icosadienyl. In a preferred embodiment, R³ and R⁴ are both linoleyl. In some embodiments, R³ and R⁴ comprise at least three sites of unsaturation and are independently selected from, e.g., dodecatrienyl, tetradectrienyl, hexadecatrienyl, linolenyl, and icosatrienyl.

The cationic lipid typically comprises from about 2 mol % to about 60 mol %, from about 5 mol % to about 50 mol %, from about 10 mol % to about 50 mol %, from about 20 mol % to about 50 mol %, from about 20 mol % to about 40 mol %, from about 30 mol % to about 40 mol %, or about 40 mol % of the total lipid present in the particle. It will be readily apparent to one of skill in the art that depending on the intended use of the particles, the proportions of the components can be varied and the delivery efficiency of a particular formulation can be measured using, e.g., an endosomal release parameter (ERP) assay. For example, for systemic delivery, the cationic lipid may comprise from about 5 mol % to about 15 mol % of the total lipid present in the particle, and for local or regional delivery, the cationic lipid may comprise from about 30 mol % to about 50 mol %, or about 40 mol % of the total lipid present in the particle.

2. Non-Cationic Lipids

The non-cationic lipids used in the stabilized nucleic acid-lipid particles of the present invention can be any of a variety of neutral uncharged, zwitterionic, or anionic lipids capable of producing a stable complex. They are preferably neutral, although they can alternatively be positively or negatively charged. Examples of non-cationic lipids include, without limitation, phospholipid-related materials such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipahnitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), and stearoyloleoyl-phosphatidylethanolamine (SOPE). Non-cationic lipids or sterols such as cholesterol may also be present. Additional nonphosphorous containing lipids include, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, diacylphosphatidylcholine, diacylphosphatidylethanolamine, and the like. Other lipids such as lysophosphatidylcholine and lysophosphatidylethanolamine may be present. Non-cationic lipids also include polyethylene glycol-based polymers such as PEG 2000, PEG 5000, and polyethylene glycol conjugated to phospholipids or to ceramides (referred to as PEG-Cer), as described in U.S. patent application Ser. No. 08/316,429.

In preferred embodiments, the non-cationic lipids are diacylphosphatidylcholine (e.g., distearoylphosphatidylcholine, dioleoylphosphatidylcholine, dipalmitoylphosphatidylcholine, and dilinoleoylphosphatidylcholine), diacylphosphatidylethanolamine (e.g., dioleoylphosphatidylethanolamine and palmitoyloleoyl-phosphatidylethanolamine), ceramide, or sphingomyelin. The acyl groups in these lipids are preferably acyl groups derived from fatty acids having C₁₀-C₂₄ carbon chains. More preferably, the acyl groups are lauroyl, myristoyl, palmitoyl, stearoyl, or oleoyl. In particularly preferred embodiments, the non-cationic lipid includes one or more of cholesterol, DOPE, or ESM.

The non-cationic lipid typically comprises from about 5 mol % to about 90 mol %, from about 10 mol % to about 85 mol %, from about 20 mol % to about 80 mol %, or about 20 mol % of the total lipid present in the particle. The particles may further comprise cholesterol. If present, the cholesterol typically comprises from about 0 mol % to about 10 mol %, from about 2 mol % to about 10 mol %, from about 10 mol % to about 60 mol %, from about 12 mol % to about 58 mol %, from about 20 mol % to about 55 mol %, from about 30 mol % to about 50 mol %, or about 48 mol % of the total lipid present in the particle.

3. Bilayer Stabilizing Component

In addition to cationic and non-cationic lipids, the stabilized nucleic acid-lipid particles of the present invention can comprise a bilayer stabilizing component (BSC) such as an ATTA-lipid or a PEG-lipid such as PEG coupled to dialkyloxypropyls (PEG-DAA) as described in, e.g., PCT Publication No. WO 05/026372, PEG coupled to diacylglycerol (PEG-DAG) as described in, e.g., U.S. Patent Publication Nos. 20030077829 and 2005008689, PEG coupled to phospholipids such as phosphatidylethanolamine (PEG-PE), PEG conjugated to ceramides, or a mixture thereof (see, e.g., U.S. Pat. No. 5,885,613). In a preferred embodiment, the BSC is a conjugated lipid that prevents the aggregation of particles. Suitable conjugated lipids include, but are not limited to, PEG-lipid conjugates, ATTA-lipid conjugates, cationic-polymer-lipid conjugates (CPLs), and mixtures thereof. In another preferred embodiment, the particles comprise either a PEG-lipid conjugate or an ATTA-lipid conjugate together with a CPL.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH₂), monomethoxypolyethylene glycol-tresylate (MePEG-TRES), and monomethoxypolyethylene glycol-imidazolyl-carbonyl (MePEG-IM). In addition, monomethoxypolyethyleneglycol-acetic acid (MePEG-CH₂COOH) is particularly useful for preparing the PEG-lipid conjugates including, e.g., PEG-DAA conjugates.

In a preferred embodiment, the PEG has an average molecular weight of from about 550 daltons to about 10,000 daltons, more preferably from about 750 daltons to about 5,000 daltons, more preferably from about 1,000 daltons to about 5,000 daltons, more preferably from about 1,500 daltons to about 3,000 daltons, and even more preferably about 2,000 daltons or about 750 daltons. The PEG can be optionally substituted by an alkyl, alkoxy, acyl, or aryl group. The PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In a preferred embodiment, the linker moiety is a non-ester containing linker moiety. As used herein, the term “non-ester containing linker moiety” refers to a linker moiety that does not contain a carboxylic ester bond (—OC(O)—). Suitable non-ester containing linker moieties include, but are not limited to, amido (—C(O)NH—), amino (—NR—), carbonyl (—C(O)—), carbamate (—NHC(O)O—), urea (—NHC(O)NH—), disulphide (—S—S—), ether (—O—), succinyl (—(O)CCH₂CH₂C(O)—), succinamidyl (—NHC(O)CH₂CH₂C(O)NH—), ether, disulphide, as well as combinations thereof (such as a linker containing both a carbamate linker moiety and an amido linker moiety). In a preferred embodiment, a carbamate linker is used to couple the PEG to the lipid.

In other embodiments, an ester containing linker moiety is used to couple the PEG to the lipid. Suitable ester containing linker moieties include, e.g., carbonate (—OC(O)O—), succinoyl, phosphate esters (—O—(O)POH—O—), sulfonate esters, and combinations thereof.

Phosphatidylethanolamines having a variety of acyl chain groups of varying chain lengths and degrees of saturation can be conjugated to PEG to form the bilayer stabilizing component. Such phosphatidylethanolamines are commercially available, or can be isolated or synthesized using conventional techniques known to those of skilled in the art. Phosphatidylethanolamines containing saturated or unsaturated fatty acids with carbon chain lengths in the range of C₁₀ to C₂₀ are preferred. Phosphatidylethanolamines with mono- or diunsaturated fatty acids and mixtures of saturated and unsaturated fatty acids can also be used. Suitable phosphatidylethanolamines include, but are not limited to, dimyristoyl-phosphatidylethanolamine (DMPE), dipalmitoyl-phosphatidylethanolamine (DPPE), dioleoylphosphatidylethanolamine (DOPE), and distearoyl-phosphatidylethanolamine (DSPE).

The term “ATTA” or “polyamide” refers to, without limitation, compounds disclosed in U.S. Pat. Nos. 6,320,017 and 6,586,559. These compounds include a compound having the formula: 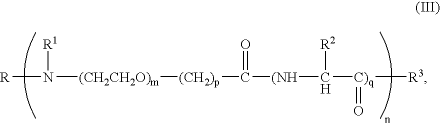
wherein R is a member selected from the group consisting of hydrogen, alkyl and acyl; R¹ is a member selected from the group consisting of hydrogen and alkyl; or optionally, R and R¹ and the nitrogen to which they are bound form an azido moiety; R² is a member of the group selected from hydrogen, optionally substituted alkyl, optionally substituted aryl and a side chain of an amino acid; R³ is a member selected from the group consisting of hydrogen, halogen, hydroxy, alkoxy, mercapto, hydrazino, amino and NR⁴R⁵, wherein R⁴ and R⁵ are independently hydrogen or alkyl; n is 4 to 80; m is 2 to 6; p is 1 to 4; and q is 0 or 1. It will be apparent to those of skill in the art that other polyamides can be used in the compounds of the present invention.

The term “diacylglycerol” refers to a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauryl (C12), myristyl (C14), palmityl (C16), stearyl (C18), and icosyl (C20). In preferred embodiments, R¹ and R² are the same, i.e., R¹ and R² are both myristyl (i.e., dimyristyl), R¹ and R² are both stearyl (i.e., distearyl), etc. Diacylglycerols have the following general formula:

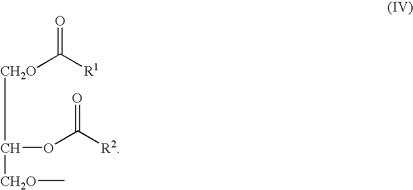

The term “dialkyloxypropyl” refers to a compound having 2 alkyl chains, R¹ and R², both of which have independently between 2 and 30 carbons. The alkyl groups can be saturated or have varying degrees of unsaturation. Dialkyloxypropyls have the following general formula:

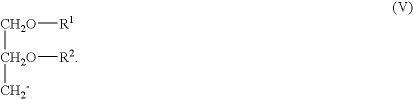

In a preferred embodiment, the PEG-lipid is a PEG-DAA conjugate having the following formula: 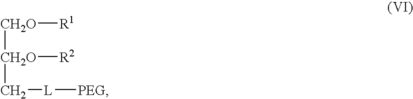
wherein R¹ and R² are independently selected and are long-chain alkyl groups having from about 10 to about 22 carbon atoms; PEG is a polyethyleneglycol; and L is a non-ester containing linker moiety or an ester containing linker moiety as described above. The long-chain alkyl groups can be saturated or unsaturated. Suitable alkyl groups include, but are not limited to, lauryl (C12), myristyl (C14), palmityl (C16), stearyl (C18), and icosyl (C20). In preferred embodiments, R¹ and R² are the same, i.e., R¹ and R² are both myristyl (i.e., dimyristyl), R¹ and R² are both stearyl (i.e., distearyl), etc.

In Formula VI above, the PEG has an average molecular weight ranging from about 550 daltons to about 10,000 daltons, more preferably from about 750 daltons to about 5,000 daltons, more preferably from about 1,000 daltons to about 5,000 daltons, more preferably from about 1,500 daltons to about 3,000 daltons, and even more preferably about 2,000 daltons or about 750 daltons. The PEG can be optionally substituted with alkyl, alkoxy, acyl, or aryl. In a preferred embodiment, the terminal hydroxyl group is substituted with a methoxy or methyl group.

In a preferred embodiment, “L” is a non-ester containing linker moiety. Suitable non-ester containing linkers include, but are not limited to, an amido linker moiety, an amino linker moiety, a carbonyl linker moiety, a carbamate linker moiety, a urea linker moiety, an ether linker moiety, a disulfide linker moiety, a succinamidyl linker moiety, and combinations thereof. In a preferred embodiment, the non-ester containing linker moiety is a carbamate linker moiety (i.e., a PEG-C-DAA conjugate). In another preferred embodiment, the non-ester containing linker moiety is an amido linker moiety (i.e., a PEG-A-DAA conjugate). In yet another preferred embodiment, the non-ester containing linker moiety is a succinamidyl linker moiety (i.e., a PEG-S-DAA conjugate).

The PEG-DAA conjugates are synthesized using standard techniques and reagents known to those of skill in the art. It will be recognized that the PEG-DAA conjugates will contain various amide, amine,- ether, thio, carbamate, and urea linkages. Those of skill in the art will recognize that methods and reagents for forming these bonds are well known and readily available. See, e.g., March, ADVANCED ORGANIC CHEMISTRY (Wiley 1992), Larock, COMPREHENSIVE ORGANIC TRANSFORMATIONS (VCH 1989); and Furniss, VOGEL'S TEXTBOOK OF PRACTICAL ORGANIC CHEMISTRY 5th ed. (Longman 1989). It will also be appreciated that any functional groups present may require protection and deprotection at different points in the synthesis of the PEG-DAA conjugates. Those of skill in the art will recognize that such techniques are well known. See, e.g., Green and Wuts, PROTECTIVE GROUPS IN ORGANIC SYNTHESIS (Wiley 1991).

Preferably, the PEG-DAA conjugate is a dilauryloxypropyl (C12)-PEG conjugate, dimyristyloxypropyl (C14)-PEG conjugate, a dipalmityloxypropyl (C16)-PEG conjugate, or a distearyloxypropyl (C18)-PEG conjugate. Those of skill in the art will readily appreciate that other dialkyloxypropyls can be used in the PEG-DAA conjugates of the present invention.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., SNALPs or SPLPs) of the present invention can further comprise cationic poly(ethylene glycol) (PEG) lipids or CPLs that have been designed for insertion into lipid bilayers to impart a positive charge(see, e.g., Chen et al., Bioconj. Chem., 11:433-437 (2000)). Suitable SPLPs and SPLP-CPLs for use in the present invention, and methods of making and using SPLPs and SPLP-CPLs, are disclosed, e.g., in U.S. Pat. No. 6,852,334 and PCT Publication No. WO 00/62813. Cationic polymer lipids (CPLs) useful in the present invention have the following architectural features: (1) a lipid anchor, such as a hydrophobic lipid, for incorporating the CPLs into the lipid bilayer; (2) a hydrophilic spacer, such as a polyethylene glycol, for linking the lipid anchor to a cationic head group; and (3) a polycationic moiety, such as a naturally occurring amino acid, to produce a protonizable cationic head group.

Suitable CPLs include compounds of Formula VII:
A-W-Y (VII),
wherein A, W, and Y are as described below.

With reference to Formula VII, “A” is a lipid moiety such as an amphipathic lipid, a neutral lipid, or a hydrophobic lipid that acts as a lipid anchor. Suitable lipid examples include vesicle-forming lipids or vesicle adopting lipids and include, but are not limited to, diacylglycerolyls, dialkylglycerolyls, N-N-dialkylaminos, 1,2-diacyloxy-3-aminopropanes, and 1,2-dialkyl-3-aminopropanes.

“W” is a polymer or an oligomer such as a hydrophilic polymer or oligomer. Preferably, the hydrophilic polymer is a biocompatable polymer that is nonimmunogenic or possesses low inherent immunogenicity. Alternatively, the hydrophilic polymer can be weakly antigenic if used with appropriate adjuvants. Suitable nonimmunogenic polymers include, but are not limited to, PEG, polyamides, polylactic acid, polyglycolic acid, polylactic acid/polyglycolic acid copolymers, and combinations thereof. In a preferred embodiment, the polymer has a molecular weight of from about 250 to about 7,000 daltons.

“Y” is a polycationic moiety. The term polycationic moiety refers to a compound, derivative, or functional group having a positive charge, preferably at least 2 positive charges at a selected pH, preferably physiological pH. Suitable polycationic moieties include basic amino acids and their derivatives such as arginine, asparagine, glutamine, lysine, and histidine; spermine; spermidine; cationic dendrimers; polyamines; polyamine sugars; and amino polysaccharides. The polycationic moieties can be linear, such as linear tetralysine, branched or dendrimeric in structure. Polycationic moieties have between about 2 to about 15 positive charges, preferably between about 2 to about 12 positive charges, and more preferably between about 2 to about 8 positive charges at selected pH values. The selection of which polycationic moiety to employ may be determined by the type of particle application which is desired.

The charges on the polycationic moieties can either be distributed around the entire particle moiety, or alternatively, they can be a discrete concentration of charge density in one particular area of the particle moiety e.g., a charge spike. If the charge density is distributed on the particle, the charge density can be equally distributed or unequally distributed. All variations of charge distribution of the polycationic moiety are encompassed by the present invention.

The lipid “A” and the nonimmunogenic polymer “W” can be attached by various methods and preferably by covalent attachment. Methods known to those of skill in the art can be used for the covalent attachment of “A” and “W.” Suitable linkages include, but are not limited to, amide, amine, carboxyl, carbonate, carbamate, ester, and hydrazone linkages. It will be apparent to those skilled in the art that “A” and “W” must have complementary functional groups to effectuate the linkage. The reaction of these two groups, one on the lipid and the other on the polymer, will provide the desired linkage. For example, when the lipid is a diacylglycerol and the terminal hydroxyl is activated, for instance with NHS and DCC, to form an active ester, and is then reacted with a polymer which contains an amino group, such as with a polyamide (see, e.g., U.S. Pat. Nos. 6,320,017 and 6,586,559), an amide bond will form between the two groups.

In certain instances, the polycationic moiety can have a ligand attached, such as a targeting ligand or a chelating moiety for complexing calcium. Preferably, after the ligand is attached, the cationic moiety maintains a positive charge. In certain instances, the ligand that is attached has a positive charge. Suitable ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

The bilayer stabilizing component (e.g., PEG-lipid) typically comprises from about 0 mol % to about 20 mol %, from about 0.5 mol % to about 20 mol %, from about 1.5 mol % to about 18 mol %, from about 4 mol % to about 15 mol %, from about 5 mol % to about 12 mol %, or about 2 mol % of the total lipid present in the particle. One of ordinary skill in the art will appreciate that the concentration of the bilayer stabilizing component can be varied depending on the bilayer stabilizing component employed and the rate at which the nucleic acid-lipid particle is to become fusogenic.

By controlling the composition and concentration of the bilayer stabilizing component, one can control the rate at which the bilayer stabilizing component exchanges out of the nucleic acid-lipid particle and, in turn, the rate at which the nucleic acid-lipid particle becomes fusogenic. For instance, when a polyethyleneglycol-phosphatidylethanolamine conjugate or a polyethyleneglycol-ceramide conjugate is used as the bilayer stabilizing component, the rate at which the nucleic acid-lipid particle becomes fusogenic can be varied, for example, by varying the concentration of the bilayer stabilizing component, by varying the molecular weight of the polyethyleneglycol, or by varying the chain length and degree of saturation of the acyl chain groups on the phosphatidylethanolamine or the ceramide. In addition, other variables including, for example, pH, temperature, ionic strength, etc. can be used to vary and/or control the rate at which the nucleic acid-lipid particle becomes fusogenic. Other methods which can be used to control the rate at which the nucleic acid-lipid particle becomes fusogenic will become apparent to those of skill in the art upon reading this disclosure.

B. Additional Carrier Systems

Non-limiting examples of additional lipid-based carrier systems suitable for use in the present invention include lipoplexes (see, e.g., U.S. Patent Publication No. 20030203865; and Zhang et al., J. Control Release, 100:165-180 (2004)), pH-sensitive lipoplexes (see, e.g., U.S. Patent Publication No. 20020192275), reversibly masked lipoplexes (see, e.g., U.S. Patent Publication Nos. 20030180950), cationic lipid-based compositions (see, e.g., U.S. Pat. No. 6,756,054; and U.S. Patent Publication No. 20050234232), cationic liposomes (see, e.g., U.S. Patent Publication Nos. 20030229040, 20020160038, and 20020012998; U.S. Pat. No. 5,908,635; and PCT Publication No. WO 01/72283), anionic liposomes (see, e.g., U.S. Patent Publication No. 20030026831), pH-sensitive liposomes (see, e.g., U.S. Patent Publication No. 20020192274; and AU 2003210303), antibody-coated liposomes (see, e.g., U.S. Patent Publication No. 20030108597; and PCT Publication No. WO 00/50008), cell-type specific liposomes (see, e.g., U.S. Patent Publication No. 20030198664), liposomes containing nucleic acid and peptides (see, e.g., U.S. Pat. No. 6,207,456), liposomes containing lipids derivatized with releasable hydrophilic polymers (see, e.g., U.S. Patent Publication No. 20030031704), lipid-entrapped nucleic acid (see, e.g., PCT Publication Nos. WO 03/057190 and WO 03/059322), lipid-encapsulated nucleic acid (see, e.g., U.S. Patent Publication No. 20030129221; and U.S. Pat. No. 5,756,122), other liposomal compositions (see, e.g., U.S. Patent Publication Nos. 20030035829 and 20030072794; and U.S. Pat. No. 6,200,599), stabilized mixtures of liposomes and emulsions (see, e.g., EP1304160), emulsion compositions (see, e.g., U.S. Pat. No. 6,747,014), and nucleic acid micro-emulsions (see, e.g., U.S. Patent Publication No. 20050037086).

Examples of polymer-based carrier systems suitable for use in the present invention include, but are not limited to, cationic polymer-nucleic acid complexes (i.e., polyplexes). To form a polyplex, a nucleic acid (e.g., siRNA) is typically complexed with a cationic polymer having a linear, branched, star, or dendritic polymeric structure that condenses the nucleic acid into positively charged particles capable of interacting with anionic proteoglycans at the cell surface and entering cells by endocytosis. In some embodiments, the polyplex comprises nucleic acid (e.g., siRNA) complexed with a cationic polymer such as polyethylenimine (PEI) (see, e.g., U.S. Pat. No. 6,013,240; commercially available from Qbiogene, Inc. (Carlsbad, Calif.) as In vivo jetPEI™, a linear form of PEI), polypropylenimine (PPI), polyvinylpyrrolidone (PVP), poly-L-lysine (PLL), diethylaminoethyl (DEAE)-dextran, poly(β-amino ester) (PAE) polymers (see, e.g., Lynn et al., J. Am. Chem. Soc., 123:8155-8156 (2001)), chitosan, polyamidoamine (PAMAM) dendrimers (see, e.g., Kukowska-Latallo et al., Proc. Natl. Acad. Sci. USA, 93:4897-4902 (1996)), porphyrin (see, e.g., U.S. Pat. No. 6,620,805), polyvinylether (see, e.g., U.S. Patent Publication No. 20040156909), polycyclic amidinium (see, e.g., U.S. Patent Publication No. 20030220289), other polymers comprising primary amine, imine, guanidine, and/or imidazole groups (see, e.g., U.S. Pat. No. 6,013,240; PCT Publication No. WO/9602655; PCT Publication No. WO95/21931; Zhang et al., J. Control Release, 100:165-180 (2004); and Tiera et al., Curr. Gene Ther., 6:59-71 (2006)), and a mixture thereof. In other embodiments, the polyplex comprises cationic polymer-nucleic acid complexes as described in U.S. Patent Publication Nos. 20060211643, 20050222064, 20030125281, and 20030185890, and PCT Publication No. WO 03/066069; biodegradable poly(β-amino ester) polymer-nucleic acid complexes as described in U.S. Patent Publication No. 20040071654; microparticles containing polymeric matrices as described in U.S. Patent Publication No. 20040142475; other microparticle compositions as described in U.S. Patent Publication No. 20030157030; condensed nucleic acid complexes as described in U.S. Patent Publication No. 20050123600; and nanocapsule and microcapsule compositions as described in AU 2002358514 and PCT Publication No. WO 02/096551.

In certain instances, the nucleic acid (e.g., siRNA) may be complexed with cyclodextrin or a polymer thereof. Non-limiting examples of cyclodextrin-based carrier systems include the cyclodextrin-modified polymer-nucleic acid complexes described in U.S. Patent Publication No. 20040087024; the linear cyclodextrin copolymer-nucleic acid complexes described in U.S. Pat. Nos. 6,509,323, 6,884,789, and 7,091,192; and the cyclodextrin polymer-complexing agent-nucleic acid complexes described in U.S. Pat. No. 7,018,609. In certain other instances, the nucleic acid (e.g., siRNA) may be complexed with a peptide or polypeptide. An example of a protein-based carrier system includes, but is not limited to, the cationic oligopeptide-nucleic acid complex described in PCT Publication No. WO95/21931.

### V. Preparation of Nucleic Acid-Lipid Particles

The serum-stable nucleic acid-lipid particles of the present invention, in which an interfering RNA (e.g., an anti-influenza siRNA) is encapsulated in a lipid bilayer and is protected from degradation, can be formed by any method known in the art including, but not limited to, a continuous mixing method, a direct dilution process, a detergent dialysis method, or a modification of a reverse-phase method which utilizes organic solvents to provide a single phase during mixing of the components.

In preferred embodiments, the cationic lipids are lipids of Formula I and II or combinations thereof. In other preferred embodiments, the non-cationic lipids are egg sphingomyelin (ESM), distearoylphosphatidylcholine (DSPC), dioleoylphosphatidylcholine (DOPC), 1-palmitoyl-2-oleoyl-phosphatidylcholine (POPC), dipalmitoyl-phosphatidylcholine (DPPC), monomethyl-phosphatidylethanolamine, dimethyl-phosphatidylethanolamine, 14:0 PE (1,2-dimyristoyl-phosphatidylethanolamine (DMPE)), 16:0 PE (1,2-dipalmitoyl-phosphatidylethanolamine (DPPE)), 18:0 PE (1,2-distearoyl-phosphatidylethanolamine (DSPE)), 18:1 PE (1,2-dioleoyl-phosphatidylethanolamine (DOPE)), 18:1 trans PE (1,2-dielaidoyl-phosphatidylethanolamine (DEPE)), 18:0-18:1 PE (1-stearoyl-2-oleoyl-phosphatidylethanolamine (SOPE)), 16:0-18:1 PE (1-palmitoyl-2-oleoyl-phosphatidylethanolamine (POPE)), polyethylene glycol-based polymers (e.g., PEG 2000, PEG 5000, PEG-modified diacylglycerols, or PEG-modified dialkyloxypropyls), cholesterol, or combinations thereof. In still other preferred embodiments, the organic solvents are methanol, chloroform, methylene chloride, ethanol, diethyl ether, or combinations thereof.

In a preferred embodiment, the present invention provides for nucleic acid-lipid particles produced via a continuous mixing method, e.g., process that includes providing an aqueous solution comprising a nucleic acid such as an siRNA in a first reservoir, providing an organic lipid solution in a second reservoir, and mixing the aqueous solution with the organic lipid solution such that the organic lipid solution mixes with the aqueous solution so as to substantially instantaneously produce a liposome encapsulating the nucleic acid (e.g., siRNA). This process and the apparatus for carrying this process are described in detail in U.S. Patent Publication No. 20040142025.

The action of continuously introducing lipid and buffer solutions into a mixing environment, such as in a mixing chamber, causes a continuous dilution of the lipid solution with the buffer solution, thereby producing a liposome substantially instantaneously upon mixing. As used herein, the phrase “continuously diluting a lipid solution with a buffer solution” (and variations) generally means that the lipid solution is diluted sufficiently rapidly in a hydration process with sufficient force to effectuate vesicle generation. By mixing the aqueous solution comprising a nucleic acid with the organic lipid solution, the organic lipid solution undergoes a continuous stepwise dilution in the presence of the buffer solution (i.e., aqueous solution) to produce a nucleic acid-lipid particle.

The serum-stable nucleic acid-lipid particles formed using the continuous mixing method typically have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

In another embodiment, the present invention provides for nucleic acid-lipid particles produced via a direct dilution process that includes forming a liposome solution and immediately and directly introducing the liposome solution into a collection vessel containing a controlled amount of dilution buffer. In preferred aspects, the collection vessel includes one or more elements configured to stir the contents of the collection vessel to facilitate dilution. In one aspect, the amount of dilution buffer present in the collection vessel is substantially equal to the volume of liposome solution introduced thereto. As a non-limiting example, a liposome solution in 45% ethanol when introduced into the collection vessel containing an equal volume of ethanol will advantageously yield smaller particles in about 22.5%, about 20%, or about 15% ethanol.

In yet another embodiment, the present invention provides for nucleic acid-lipid particles produced via a direct dilution process in which a third reservoir containing dilution buffer is fluidly coupled to a second mixing region. In this embodiment, the liposome solution formed in a first mixing region is immediately and directly mixed with dilution buffer in the second mixing region. In preferred aspects, the second mixing region includes a T-connector arranged so that the liposome solution and the dilution buffer flows meet as opposing 180° flows; however, connectors providing shallower angles can be used, e.g., from about 27° to about 180°. A pump mechanism delivers a controllable flow of buffer to the second mixing region. In one aspect, the flow rate of dilution buffer provided to the second mixing region is controlled to be substantially equal to the flow rate of liposome solution introduced thereto from the first mixing region. This embodiment advantageously allows for more control of the flow of dilution buffer mixing with the liposome solution in the second mixing region, and therefore also the concentration of liposome solution in buffer throughout the second mixing process. Such control of the dilution buffer flow rate advantageously allows for small particle size formation at reduced concentrations.

These processes and the apparatuses for carrying out these direct dilution processes is described in detail in U.S. patent application Ser. No. 11/495,150.

The serum-stable nucleic acid-lipid particles formed using the direct dilution process typically have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

In some embodiments, the particles are formed using detergent dialysis. Without intending to be bound by any particular mechanism of formation, a nucleic acid such as an siRNA is contacted with a detergent solution of cationic lipids to form a coated nucleic acid complex. These coated nucleic acids can aggregate and precipitate. However, the presence of a detergent reduces this aggregation and allows the coated nucleic acids to react with excess lipids (typically, non-cationic lipids) to form particles in which the nucleic acid is encapsulated in a lipid bilayer. Thus, the serum-stable nucleic acid-lipid particles can be prepared as follows:
(a) combining a nucleic acid with cationic lipids in a detergent solution to form a coated nucleic acid-lipid complex;
(b) contacting non-cationic lipids with the coated nucleic acid-lipid complex to form a detergent solution comprising a nucleic acid-lipid complex and non-cationic lipids; and
(c) dialyzing the detergent solution of step (b) to provide a solution of serum-stable nucleic acid-lipid particles, wherein the nucleic acid is encapsulated in a lipid bilayer and the particles are serum-stable and have a size of from about 50 to about 150 nm.

An initial solution of coated nucleic acid-lipid complexes is formed by combining the nucleic acid with the cationic lipids in a detergent solution. In these embodiments, the detergent solution is preferably an aqueous solution of a neutral detergent having a critical micelle concentration of 15-300 mM, more preferably 20-50 mM. Examples of suitable detergents include, for example, N,N′-((octanoylimino)-bis-(trimethylene))-bis-(D-gluconamide) (BIGCHAP); BRIJ 35; Deoxy-BIGCHAP; dodecylpoly(ethylene glycol) ether; Tween 20; Tween 40; Tween 60; Tween 80; Tween 85; Mega 8; Mega 9; Zwittergent® 3-08; Zwittergent® 3-10; Triton X-405; hexyl-, heptyl-, octyl- and nonyl-β-D-glucopyranoside; and heptylthioglucopyranoside; with octyl β-D-glucopyranoside and Tween-20 being the most preferred. The concentration of detergent in the detergent solution is typically about 100 mM to about 2 M, preferably from about 200 mM to about 1.5 M.

The cationic lipids and nucleic acids will typically be combined to produce a charge ratio (+/−) of about 1:1 to about 20:1, in a ratio of about 1:1 to about 12:1, or in a ratio of about 2:1 to about 6:1. Additionally, the overall concentration of nucleic acid in solution will typically be from about 25 μg/ml to about 1 mg/ml, from about 25 μg/ml to about 200 μg/ml, or from about 50 μg/ml to about 100 μg/ml. The combination of nucleic acids and cationic lipids in detergent solution is kept, typically at room temperature, for a period of time which is sufficient for the coated complexes to form. Alternatively, the nucleic acids and cationic lipids can be combined in the detergent solution and warmed to temperatures of up to about 37° C., about 50° C., about 60° C., or about 70° C. For nucleic acids which are particularly sensitive to temperature, the coated complexes can be formed at lower temperatures, typically down to about 4° C.

In some embodiments, the nucleic acid to lipid ratios (mass/mass ratios) in a formed nucleic acid-lipid particle will range from about 0.01 to about 0.2, from about 0.02 to about 0.1, from about 0.03 to about 0.1, or from about 0.01 to about 0.08. The ratio of the starting materials also falls within this range. In other embodiments, the nucleic acid-lipid particle preparation uses about 400 μg nucleic acid per 10 mg total lipid or a nucleic acid to lipid mass ratio of about 0.01 to about 0.08 and, more preferably, about 0.04, which corresponds to 1.25 mg of total lipid per 50 μg of nucleic acid. In other preferred embodiments, the particle has a nucleic acid:lipid mass ratio of about 0.08.

The detergent solution of the coated nucleic acid-lipid complexes is then contacted with non-cationic lipids to provide a detergent solution of nucleic acid-lipid complexes and non-cationic lipids. The non-cationic lipids which are useful in this step include, diacylphosphatidylcholine, diacylphosphatidylethanolamine, ceramide, sphingomyelin, cephalin, cardiolipin, and cerebrosides. In preferred embodiments, the non-cationic lipids are diacylphosphatidylcholine, diacylphosphatidylethanolamine, ceramide, or sphingomyelin. The acyl groups in these lipids are preferably acyl groups derived from fatty acids having C₁₀-C₂₄ carbon chains. More preferably, the acyl groups are lauroyl, myristoyl, palmitoyl, stearoyl, or oleoyl. In particularly preferred embodiments, the non-cationic lipids are DSPC, DOPE, POPC, egg phosphatidylcholine (EPC), cholesterol, or a mixture thereof. In the most preferred embodiments, the nucleic acid-lipid particles are fusogenic particles with enhanced properties in vivo and the non-cationic lipid is DSPC or DOPE. In addition, the nucleic acid-lipid particles of the present invention may further comprise cholesterol. In other preferred embodiments, the non-cationic lipids can further comprise polyethylene glycol-based polymers such as PEG 2,000, PEG 5,000, and PEG conjugated to a diacylglycerol, a ceramide, or a phospholipid, as described in, e.g., U.S. Pat. No. 5,820,873 and U.S. Patent Publication No. 20030077829. In further preferred embodiments, the non-cationic lipids can further comprise polyethylene glycol-based polymers such as PEG 2,000, PEG 5,000, and PEG conjugated to a dialkyloxypropyl.

The amount of non-cationic lipid which is used in the present methods is typically from about 2 to about 20 mg of total lipids to 50 μg of nucleic acid. Preferably, the amount of total lipid is from about 5 to about 10 mg per 50 μg of nucleic acid.

Following formation of the detergent solution of nucleic acid-lipid complexes and non-cationic lipids, the detergent is removed, preferably by dialysis. The removal of the detergent results in the formation of a lipid-bilayer which surrounds the nucleic acid providing serum-stable nucleic acid-lipid particles which have a size of from about 50 nm to about 150 nm, from about 60 nm to about 130 nm, from about 70 nm to about 110 nm, or from about 70 nm to about 90 nm. The particles thus formed do not aggregate and are optionally sized to achieve a uniform particle size.

The serum-stable nucleic acid-lipid particles can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles, is described in U.S. Pat. No. 4,737,323. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and about 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In another group of embodiments, the serum-stable nucleic acid-lipid particles can be prepared as follows:
(a) preparing a mixture comprising cationic lipids and non-cationic lipids in an organic solvent;
(b) contacting an aqueous solution of nucleic acid with the mixture in step (a) to provide a clear single phase; and
(c) removing the organic solvent to provide a suspension of nucleic acid-lipid particles, wherein the nucleic acid is encapsulated in a lipid bilayer and the particles are stable in serum and have a size of from about 50 to about 150 nm.

The nucleic acids (e.g., siRNA), cationic lipids, and non-cationic lipids which are useful in this group of embodiments are as described for the detergent dialysis methods above.

The selection of an organic solvent will typically involve consideration of solvent polarity and the ease with which the solvent can be removed at the later stages of particle formation. The organic solvent, which is also used as a solubilizing agent, is in an amount sufficient to provide a clear single phase mixture of nucleic acid and lipids. Suitable solvents include, but are not limited to, chloroform, dichloromethane, diethylether, cyclohexane, cyclopentane, benzene, toluene, methanol, or other aliphatic alcohols such as propanol, isopropanol, butanol, tert-butanol, iso-butanol, pentanol and hexanol. Combinations of two or more solvents may also be used in the present invention.

Contacting the nucleic acid with the organic solution of cationic and non-cationic lipids is accomplished by mixing together a first solution of nucleic acid, which is typically an aqueous solution, and a second organic solution of the lipids. One of skill in the art will understand that this mixing can take place by any number of methods, for example, by mechanical means such as by using vortex mixers.

After the nucleic acid has been contacted with the organic solution of lipids, the organic solvent is removed, thus forming an aqueous suspension of serum-stable nucleic acid-lipid particles. The methods used to remove the organic solvent will typically involve evaporation at reduced pressures or blowing a stream of inert gas (e.g., nitrogen or argon) across the mixture.

The serum-stable nucleic acid-lipid particles thus formed will typically be sized from about 50 nm to about 150 nm, from about 60 nm to about 130 nm, from about 70 nm to about 110 nm, or from about 70 nm to about 90 nm. To achieve further size reduction or homogeneity of size in the particles, sizing can be conducted as described above.

In other embodiments, the methods will further comprise adding non-lipid polycations which are useful to effect the delivery to cells using the present compositions. Examples of suitable non-lipid polycations include, but are limited to, hexadimethrine bromide (sold under the brand name POLYBRENE®, from Aldrich Chemical Co., Milwaukee, Wis., USA) or other salts of heaxadimethrine. Other suitable polycations include, for example, salts of poly-L-ornithine, poly-L-arginine, poly-L-lysine, poly-D-lysine, polyallylamine, and polyethyleneimine.

In certain embodiments, the formation of the nucleic acid-lipid particles can be carried out either in a mono-phase system (e.g., a Bligh and Dyer monophase or similar mixture of aqueous and organic solvents) or in a two-phase system with suitable mixing.

When formation of the complexes is carried out in a mono-phase system, the cationic lipids and nucleic acids are each dissolved in a volume of the mono-phase mixture. Combination of the two solutions provides a single mixture in which the complexes form. Alternatively, the complexes can form in two-phase mixtures in which the cationic lipids bind to the nucleic acid (which is present in the aqueous phase), and “pull” it into the organic phase.

In another embodiment, the serum-stable nucleic acid-lipid particles can be prepared as follows:
(a) contacting nucleic acids with a solution comprising non-cationic lipids and a detergent to form a nucleic acid-lipid mixture;
(b) contacting cationic lipids with the nucleic acid-lipid mixture to neutralize a portion of the negative charge of the nucleic acids and form a charge-neutralized mixture of nucleic acids and lipids; and
(c) removing the detergent from the charge-neutralized mixture to provide the nucleic acid-lipid particles in which the nucleic acids are protected from degradation.

In one group of embodiments, the solution of non-cationic lipids and detergent is an aqueous solution. Contacting the nucleic acids with the solution of non-cationic lipids and detergent is typically accomplished by mixing together a first solution of nucleic acids and a second solution of the lipids and detergent. One of skill in the art will understand that this mixing can take place by any number of methods, for example, by mechanical means such as by using vortex mixers. Preferably, the nucleic acid solution is also a detergent solution. The amount of non-cationic lipid which is used in the present method is typically determined based on the amount of cationic lipid used, and is typically of from about 0.2 to about 5 times the amount of cationic lipid, preferably from about 0.5 to about 2 times the amount of cationic lipid used.

In some embodiments, the nucleic acids are precondensed as described in, e.g., U.S. patent application Ser. No. 09/744,103.

The nucleic acid-lipid mixture thus formed is contacted with cationic lipids to neutralize a portion of the negative charge which is associated with the nucleic acids (or other polyanionic materials) present. The amount of cationic lipids used will typically be sufficient to neutralize at least 50% of the negative charge of the nucleic acid. Preferably, the negative charge will be at least 70% neutralized, more preferably at least 90% neutralized. Cationic lipids which are useful in the present invention, include, for example, DLinDMA and DLenDMA. These lipids and related analogs are described in U.S. Patent Publication No. 20060083780.

Contacting the cationic lipids with the nucleic acid-lipid mixture can be accomplished by any of a number of techniques, preferably by mixing together a solution of the cationic lipid and a solution containing the nucleic acid-lipid mixture. Upon mixing the two solutions (or contacting in any other manner), a portion of the negative charge associated with the nucleic acid is neutralized. Nevertheless, the nucleic acid remains in an uncondensed state and acquires hydrophilic characteristics.

After the cationic lipids have been contacted with the nucleic acid-lipid mixture, the detergent (or combination of detergent and organic solvent) is removed, thus forming the nucleic acid-lipid particles. The methods used to remove the detergent will typically involve dialysis. When organic solvents are present, removal is typically accomplished by evaporation at reduced pressures or by blowing a stream of inert gas (e.g., nitrogen or argon) across the mixture.

The particles thus formed will typically be sized from about 50 nm to several microns, about 50 nm to about 150 nm, from about 60 nm to about 130 nm, from about 70 nm to about 110 nm, or from about 70 nm to about 90 nm. To achieve further size reduction or homogeneity of size in the particles, the nucleic acid-lipid particles can be sonicated, filtered, or subjected to other sizing techniques which are used in liposomal formulations and are known to those of skill in the art.

In other embodiments, the methods will further comprise adding non-lipid polycations which are useful to effect the lipofection of cells using the present compositions. Examples of suitable non-lipid polycations include, hexadimethrine bromide (sold under the brandname POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine, and polyethyleneimine. Addition of these salts is preferably after the particles have been formed.

In another aspect, the serum-stable nucleic acid-lipid particles can be prepared as follows:
(a) contacting an amount of cationic lipids with nucleic acids in a solution; the solution comprising from about 15-35% water and about 65-85% organic solvent and the amount of cationic lipids being sufficient to produce a +/− charge ratio of from about 0.85 to about 2.0, to provide a hydrophobic nucleic acid-lipid complex;
(b) contacting the hydrophobic, nucleic acid-lipid complex in solution with non-cationic lipids, to provide a nucleic acid-lipid mixture; and
(c) removing the organic solvents from the nucleic acid-lipid mixture to provide nucleic acid-lipid particles in which the nucleic acids are protected from degradation.

The nucleic acids (e.g., siRNA), non-cationic lipids, cationic lipids, and organic solvents which are useful in this aspect of the invention are the same as those described for the methods above which used detergents. In one group of embodiments, the solution of step (a) is a mono-phase. In another group of embodiments, the solution of step (a) is two-phase.

In preferred embodiments, the non-cationic lipids are ESM, DSPC, DOPC, POPC, DPPC, monomethyl-phosphatidylethanolamine, dimethyl-phosphatidylethanolamine, DMPE, DPPE, DSPE, DOPE, DEPE, SOPE, POPE, PEG-based polymers (e.g., PEG 2000, PEG 5000, PEG-modified diacylglycerols, or PEG-modified dialkyloxypropyls), cholesterol, or combinations thereof. In still other preferred embodiments, the organic solvents are methanol, chloroform, methylene chloride, ethanol, diethyl ether or combinations thereof.

In one embodiment, the nucleic acid is an siRNA as described herein; the cationic lipid is DLindMA, DLenDMA, DODAC, DDAB, DOTMA, DOSPA, DMRIE, DOGS, or combinations thereof; the non-cationic lipid is ESM, DOPE, PEG-DAG, DSPC, DPPC, DPPE, DMPE, monomethyl-phosphatidylethanolamine, dimethyl-phosphatidylethanolamine, DSPE, DEPE, SOPE, POPE, cholesterol, or combinations thereof (e.g., DSPC and PEG-DAA); and the organic solvent is methanol, chloroform, methylene chloride, ethanol, diethyl ether or combinations thereof.

As above, contacting the nucleic acids with the cationic lipids is typically accomplished by mixing together a first solution of nucleic acids and a second solution of the lipids, preferably by mechanical means such as by using vortex mixers. The resulting mixture contains complexes as described above. These complexes are then converted to particles by the addition of non-cationic lipids and the removal of the organic solvent. The addition of the non-cationic lipids is typically accomplished by simply adding a solution of the non-cationic lipids to the mixture containing the complexes. A reverse addition can also be used. Subsequent removal of organic solvents can be accomplished by methods known to those of skill in the art and also described above.

The amount of non-cationic lipids which is used in this aspect of the invention is typically an amount of from about 0.2 to about 15 times the amount (on a mole basis) of cationic lipids which was used to provide the charge-neutralized nucleic acid-lipid complex. Preferably, the amount is from about 0.5 to about 9 times the amount of cationic lipids used.

In one embodiment, the nucleic acid-lipid particles preparing according to the above-described methods are either net charge neutral or carry an overall charge which provides the particles with greater gene lipofection activity. Preferably, the nucleic acid component of the particles is a nucleic acid which interferes with the production of an undesired protein. In other preferred embodiments, the non-cationic lipid may further comprise cholesterol.

A variety of general methods for making SNALP-CPLs (CPL-containing SNALPs) are discussed herein. Two general techniques include “post-insertion” technique, that is, insertion of a CPL into for example, a pre-formed SNALP, and the “standard” technique, wherein the CPL is included in the lipid mixture during for example, the SNALP formation steps. The post-insertion technique results in SNALPs having CPLs mainly in the external face of the SNALP bilayer membrane, whereas standard techniques provide SNALPs having CPLs on both internal and external faces. The method is especially useful for vesicles made from phospholipids (which can contain cholesterol) and also for vesicles containing PEG-lipids (such as PEG-DAAs and PEG-DAGs). Methods of making SNALP-CPL, are taught, for example, in U.S. Pat. Nos. 5,705,385; 6,586,410; 5,981,501; 6,534,484; and 6,852,334; U.S. Patent Publication No. 20020072121; and PCT Publication No. WO 00/62813.

### VI. Kits

The present invention also provides nucleic acid-lipid particles in kit form. The kit may comprise a container which is compartmentalized for holding the various elements of the nucleic acid-lipid particles (e.g., the nucleic acids and the individual lipid components of the particles). In some embodiments, the kit may further comprise an endosomal membrane destabilizer (e.g., calcium ions). The kit typically contains the nucleic acid-lipid particle compositions of the present invention, preferably in dehydrated form, with instructions for their rehydration and administration. In certain instances, the particles and/or compositions comprising the particles may have a targeting moiety attached to the surface of the particle. Methods of attaching targeting moieties (e.g., antibodies, proteins) to lipids (such as those used in the present particles) are known to those of skill in the art.

### VII. Administration of Nucleic Acid-Lipid Particles

Once formed, the serum-stable nucleic acid-lipid particles of the present invention are useful for the introduction of nucleic acids (i.e., siRNA that silences expression of an influenza gene) into cells. Accordingly, the present invention also provides methods for introducing nucleic acids (e.g., siRNA) into a cell (e.g., a lung macrophage such as an alveolar macrophage, a lung epithelial cell such as an aveolar type II cell, a lung endothelial cell, a lung fibroblast, a lung smooth muscle cell, etc.). The methods are carried out in vitro or in vivo by first forming the particles as described above and then contacting the particles with the cells for a period of time sufficient for delivery of the nucleic acid to the cells to occur.

The nucleic acid-lipid particles of the present invention can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

The nucleic acid-lipid particles of the present invention can be administered either alone or in a mixture with a pharmaceutically-acceptable carrier (e.g., physiological saline or phosphate buffer) selected in accordance with the route of administration and standard pharmaceutical practice. Generally, normal buffered saline (e.g., 135-150 mM NaCl) will be employed as the pharmaceutically-acceptable carrier. Other suitable carriers include, e.g., water, buffered water, 0.4% saline, 0.3% glycine, and the like, including glycoproteins for enhanced stability, such as albumin, lipoprotein, globulin, etc. Additional suitable carriers are described in, e.g., REMINGTON'S PHARMACEUTICAL SCIENCES, Mack Publishing Company, Philadelphia, Pa., 17th ed. (1985). As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The phrase “pharmaceutically-acceptable” refers to molecular entities and compositions that do not produce an allergic or similar untoward reaction when administered to a human.

The pharmaceutically-acceptable carrier is generally added following particle formation. Thus, after the particle is formed, the particle can be diluted into pharmaceutically-acceptable carriers such as normal buffered saline.

The concentration of particles in the pharmaceutical formulations can vary widely, i.e., from less than about 0.05%, usually at or at least about 2 to 5%, to as much as about 10 to 90% by weight, and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

The pharmaceutical compositions of the present invention may be sterilized by conventional, well-known sterilization techniques. Aqueous solutions can be packaged for use or filtered under aseptic conditions and lyophilized, the lyophilized preparation being combined with a sterile aqueous solution prior to administration. The compositions can contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

A. In Vivo Administration

Systemic delivery for in vivo therapy, i.e., delivery of a therapeutic nucleic acid to a distal target cell via body systems such as the circulation, has been achieved using nucleic acid-lipid particles such as those disclosed in PCT Publication No. WO 96/40964 and U.S. Pat. Nos. 5,705,385; 5,976,567; 5,981,501; and 6,410,328. This latter format provides a fully encapsulated nucleic acid-lipid particle that protects the nucleic acid from nuclease degradation in serum, is nonimmunogenic, is small in size, and is suitable for repeat dosing.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intraarticularly, intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). Intracellular nucleic acid delivery has also been discussed in Straubringer et al., Methods Enzymol., 101:512 (1983); Mannino et al., Biotechniques, 6:682 (1988); Nicolau et al., Crit. Rev. Ther. Drug Carrier Syst., 6:239 (1989); and Behr, Acc. Chem. Res., 26:274 (1993). Still other methods of administering lipid-based therapeutics are described in, for example, U.S. Pat. Nos. 3,993,754; 4,145,410; 4,235,871; 4,224,179; 4,522,803; and 4,588,578. The lipid-nucleic acid particles can be administered by direct injection at the site of disease or by injection at a site distal from the site of disease (see, e.g., Culver, HUMAN GENE THERAPY, MaryAnn Liebert, Inc., Publishers, New York. pp. 70-71(1994)).

The compositions of the present invention, either alone or in combination with other suitable components, can be made into aerosol formulations (i.e., they can be “nebulized”) to be administered via inhalation (e.g., intranasally or intratracheally) (see, Brigham et al., Am. J. Sci., 298:278 (1989)). Aerosol formulations can be placed into pressurized acceptable propellants, such as dichlorodifluoromethane, propane, nitrogen, and the like.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions are preferably administered, for example, by intravenous infusion, orally, topically, intraperitoneally, intravesically, or intrathecally.

Generally, when administered intravenously, the nucleic acid-lipid formulations are formulated with a suitable pharmaceutical carrier. Many pharmaceutically acceptable carriers may be employed in the compositions and methods of the present invention. Suitable formulations for use in the present invention are found, for example, in REMINGTON'S PHARMACEUTICAL SCIENCES, Mack Publishing Company, Philadelphia, Pa., 17th ed. (1985). A variety of aqueous carriers may be used, for example, water, buffered water, 0.4% saline, 0.3% glycine, and the like, and may include glycoproteins for enhanced stability, such as albumin, lipoprotein, globulin, etc. Generally, normal buffered saline (135-150 mM NaCl) will be employed as the pharmaceutically acceptable carrier, but other suitable carriers will suffice. These compositions can be sterilized by conventional liposomal sterilization techniques, such as filtration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. These compositions can be sterilized using the techniques referred to above or, alternatively, they can be produced under sterile conditions. The resulting aqueous solutions may be packaged for use or filtered under aseptic conditions and lyophilized, the lyophilized preparation being combined with a sterile aqueous solution prior to administration.

In certain applications, the nucleic acid-lipid particles disclosed herein may be delivered via oral administration to the individual. The particles may be incorporated with excipients and used in the form of ingestible tablets, buccal tablets, troches, capsules, pills, lozenges, elixirs, mouthwash, suspensions, oral sprays, syrups, wafers, and the like (see, e.g., U.S. Pat. Nos. 5,641,515, 5,580,579, and 5,792,451). These oral dosage forms may also contain the following: binders, gelatin; excipients, lubricants, and/or flavoring agents. When the unit dosage form is a capsule, it may contain, in addition to the materials described above, a liquid carrier. Various other materials may be present as coatings or to otherwise modify the physical form of the dosage unit. Of course, any material used in preparing any unit dosage form should be pharmaceutically pure and substantially non-toxic in the amounts employed.

Typically, these oral formulations may contain at least about 0.1% of the nucleic acid-lipid particles or more, although the percentage of the particles may, of course, be varied and may conveniently be between about 1% or 2% and about 60% or 70% or more of the weight or volume of the total formulation. Naturally, the amount of particles in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Formulations suitable for oral administration can consist of: (a) liquid solutions, such as an effective amount of the packaged nucleic acid (e.g., siRNA) suspended in diluents such as water, saline, or PEG 400; (b) capsules, sachets, or tablets, each containing a predetermined amount of the nucleic acid (e.g., siRNA), as liquids, solids, granules, or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the nucleic acid (e.g., siRNA) in a flavor, e.g., sucrose, as well as pastilles comprising the nucleic acid (e.g., siRNA) in an inert base, such as gelatin and glycerin or sucrose and acacia emulsions, gels, and the like containing, in addition to the nucleic acid (e.g., siRNA), carriers known in the art.

In another example of their use, nucleic acid-lipid particles can be incorporated into a broad range of topical dosage forms. For instance, the suspension containing the nucleic acid-lipid particles can be formulated and administered as gels, oils, emulsions, topical creams, pastes, ointments, lotions, foams, mousses, and the like.

When preparing pharmaceutical preparations of the nucleic acid-lipid particles of the invention, it is preferable to use quantities of the particles which have been purified to reduce or eliminate empty particles or particles with nucleic acid associated with the external surface.

The methods of the present invention may be practiced in a variety of hosts. Preferred hosts include mammalian species, such as avian (e.g., ducks), primates (e.g., humans and chimpanzees as well as other nonhuman primates), canines, felines, equines, bovines, ovines, caprines, rodents (e.g., rats and mice), lagomorphs, and swine.

The amount of particles administered will depend upon the ratio of nucleic acid to lipid, the particular nucleic acid used, the disease state being diagnosed, the age, weight, and condition of the patient, and the judgment of the clinician, but will generally be between about 0.01 and about 50 mg per kilogram of body weight, preferably between about 0.1 and about 5 mg/kg of body weight, or about 10⁸-10¹⁰ particles per administration (e.g., injection).

B. In Vitro Administration

For in vitro applications, the delivery of nucleic acids (e.g., siRNA) can be to any cell grown in culture, and of any tissue or type. In preferred embodiments, the cells are animal cells, more preferably mammalian cells, and most preferably human cells.

Contact between the cells and the nucleic acid-lipid particles, when carried out in vitro, takes place in a biologically compatible medium. The concentration of particles varies widely depending on the particular application, but is generally between about 1 μmol and about 10 mmol. Treatment of the cells with the nucleic acid-lipid particles is generally carried out at physiological temperatures (about 37° C.) for periods of time of from about 1 to 48 hours, preferably of from about 2 to 4 hours.

In one group of preferred embodiments, a nucleic acid-lipid particle suspension is added to 60-80% confluent plated cells having a cell density of from about 10³ to about 10⁵ cells/ml, more preferably about 2×10⁴ cells/ml. The concentration of the suspension added to the cells is preferably of from about 0.01 to 0.2 μg/ml, more preferably about 0.1 μg/ml.

Using an Endosomal Release Parameter (ERP) assay, the delivery efficiency of the SNALP or other lipid-based carrier system can be optimized. An ERP assay is described in detail in U.S. Patent Publication No. 20030077829. More particularly, the purpose of an ERP assay is to distinguish the effect of various cationic lipids and helper lipid components of SNALPs based on their relative effect on binding/uptake or fusion with/destabilization of the endosomal membrane. This assay allows one to determine quantitatively how each component of the SNALP or other lipid-based carrier system affects delivery efficiency, thereby optimizing the SNALPs or other lipid-based carrier systems. Usually, an ERP assay measures expression of a reporter protein (e.g., luciferase, β-galactosidase, green fluorescent protein (GFP), etc.), and in some instances, a SNALP formulation optimized for an expression plasmid will also be appropriate for encapsulating an interfering RNA. In other instances, an ERP assay can be adapted to measure downregulation of transcription or translation of a target sequence in the presence or absence of an interfering RNA (e.g., siRNA). By comparing the ERPs for each of the various SNALPs or other lipid-based formulations, one can readily determine the optimized system, e.g., the SNALP or other lipid-based formulation that has the greatest uptake in the cell.

C. Cells for Delivery of Interfering RNA

The compositions and methods of the present invention are used to treat a wide variety of cell types, in vivo and in vitro. Suitable cells include, e.g., cells of the airways, macrophages (e.g., lung macrophages such as alveolar macrophages), epithelial cells (e.g., epithelial cells in the lungs and trachea such as aveolar type II cells), fibroblasts (e.g., lung fibroblasts), endothelial cells (e.g., lung endothelial cells), smooth muscle cells (e.g., lung smooth muscle cells), hematopoietic precursor (stem) cells, keratinocytes, hepatocytes, skeletal muscle cells, osteoblasts, neurons, quiescent lymphocytes, terminally differentiated cells, slow or noncycling primary cells, parenchymal cells, lymphoid cells, bone cells, and the like.

In vivo delivery of nucleic acid-lipid particles encapsulating an interfering RNA (e.g., siRNA) is suited for targeting cells of any cell type. The methods and compositions can be employed with cells of a wide variety of vertebrates, including mammals, such as, e.g., canines, felines, equines, bovines, ovines, caprines, rodents (e.g., mice, rats, and guinea pigs), lagomorphs, swine, and primates (e.g. monkeys, chimpanzees, and humans).

To the extent that tissue culture of cells may be required, it is well-known in the art. For example, Freshney, Culture of Animal Cells, a Manual of Basic Technique, 3rd Ed., Wiley-Liss, New York (1994), Kuchler et al., Biochemical Methods in Cell Culture and Virology, Dowden, Hutchinson and Ross, Inc. (1977), and the references cited therein provide a general guide to the culture of cells. Cultured cell systems often will be in the form of monolayers of cells, although cell suspensions are also used.

D. Detection of SNALPs

In some embodiments, the nucleic acid-lipid particles are detectable in the subject at about 8, 12, 24, 48, 60, 72, or 96 hours, or 6, 8, 10, 12, 14, 16, 18, 19, 22, 24, 25, or 28 days after administration of the particles. The presence of the particles can be detected in the cells, tissues, or other biological samples from the subject. The particles may be detected, e.g., by direct detection of the particles, detection of the interfering RNA (e.g., siRNA) sequence, detection of the target sequence of interest (i.e., by detecting expression or reduced expression of the influenza gene sequence of interest), detection of influenza viral load in the subject, or a combination thereof.

1. Detection of Particles

Nucleic acid-lipid particles can be detected using any method known in the art. For example, a label can be coupled directly or indirectly to a component of the SNALP or other carrier system using methods well-known in the art. A wide variety of labels can be used, with the choice of label depending on sensitivity required, ease of conjugation with the SNALP component, stability requirements, and available instrumentation and disposal provisions. Suitable labels include, but are not limited to, spectral labels such as fluorescent dyes (e.g., fluorescein and derivatives, such as fluorescein isothiocyanate (FITC) and Oregon Green™; rhodamine and derivatives such Texas red, tetrarhodimine isothiocynate (TRITC), etc., digoxigenin, biotin, phycoerythrin, AMCA, CyDyes™, and the like; radiolabels such as ³H, ¹²⁵I, ³⁵S, ¹⁴C, ³²P, ³³P, etc.; enzymes such as horse radish peroxidase, alkaline phosphatase, etc.; spectral colorimetric labels such as colloidal gold or colored glass or plastic beads such as polystyrene, polypropylene, latex, etc. The label can be detected using any means known in the art.

2. Detection of Nucleic Acids

Nucleic acids (e.g., siRNA) are detected and quantified herein by any of a number of means well-known to those of skill in the art. The detection of nucleic acids proceeds by well-known methods such as Southern analysis, Northern analysis, gel electrophoresis, PCR, radiolabeling, scintillation counting, and affinity chromatography. Additional analytic biochemical methods such as spectrophotometry, radiography, electrophoresis, capillary electrophoresis, high performance liquid chromatography (HPLC), thin layer chromatography (TLC), and hyperdiffusion chromatography may also be employed.

The selection of a nucleic acid hybridization format is not critical. A variety of nucleic acid hybridization formats are known to those skilled in the art. For example, common formats include sandwich assays and competition or displacement assays. Hybridization techniques are generally described in, e.g., “Nucleic Acid Hybridization, A Practical Approach,” Eds. Hames and Higgins, IRL Press (1985).

The sensitivity of the hybridization assays may be enhanced through use of a nucleic acid amplification system which multiplies the target nucleic acid being detected. In vitro amplification techniques suitable for amplifying sequences for use as molecular probes or for generating nucleic acid fragments for subsequent subcloning are known. Examples of techniques sufficient to direct persons of skill through such in vitro amplification methods, including the polymerase chain reaction (PCR) the ligase chain reaction (LCR), Qβ-replicase amplification and other RNA polymerase mediated techniques (e.g., NASBA™) are found in Sambrook et al., In Molecular Cloning: A Laboratory Manual, Cold Spring Harbor Laboratory Press (2000); and Ausubel et al., SHORT PROTOCOLS IN MOLECULAR BIOLOGY, eds., Current Protocols, Greene Publishing Associates, Inc. and John Wiley & Sons, Inc. (2002); as well as U.S. Pat. No. 4,683,202; PCR Protocols, A Guide to Methods and Applications (Innis et al. eds.) Academic Press Inc. San Diego, Calif. (1990); Arnheim & Levinson (Oct. 1, 1990), C&EN 36; The Journal Of NIH Research, 3:81 (1991); Kwoh et al., Proc. Natl. Acad. Sci. USA, 86:1173 (1989); Guatelli et al., Proc. Natl. Acad. Sci. USA, 87:1874 (1990); Lomell et al., J. Clin. Chem., 35:1826 (1989); Landegren et al., Science, 241:1077 (1988); Van Brunt, Biotechnology, 8:291 (1990); Wu and Wallace, Gene, 4:560 (1989); Barringer et al., Gene, 89:117 (1990); and Sooknanan and Malek, Biotechnology, 13:563 (1995). Improved methods of cloning in vitro amplified nucleic acids are described in U.S. Pat. No. 5,426,039. Other methods described in the art are the nucleic acid sequence based amplification (NASBA™, Cangene, Mississauga, Ontario) and Qβ-replicase systems. These systems can be used to directly identify mutants where the PCR or LCR primers are designed to be extended or ligated only when a select sequence is present. Alternatively, the select sequences can be generally amplified using, for example, nonspecific PCR primers and the amplified target region later probed for a specific sequence indicative of a mutation.

Nucleic acids for use as probes, e.g., in in vitro amplification methods, for use as gene probes, or as inhibitor components are typically synthesized chemically according to the solid phase phosphoramidite triester method described by Beaucage et al., Tetrahedron Letts., 22:1859 1862 (1981), e.g., using an automated synthesizer, as described in Needham VanDevanter et al., Nucleic Acids Res., 12:6159 (1984). Purification of polynucleotides, where necessary, is typically performed by either native acrylamide gel electrophoresis or by anion exchange HPLC as described in Pearson et al., J. Chrom., 255:137 149 (1983). The sequence of the synthetic polynucleotides can be verified using the chemical degradation method of Maxam and Gilbert, In Methods in Enzymology, Grossman and Moldave (eds.), Academic Press, New York, 65:499 (1980).

An alternative means for determining the level of transcription is in situ hybridization. In situ hybridization assays are well-known and are generally described in Angerer et al., Methods Enzymol., 152:649 (1987). In an in situ hybridization assay, cells are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

E. Detection of Influenza Viral Load

Influenza viral load can be detected using any means known in the art. Typically, influenza viral load is detected in a biological sample from the subject. For example, viral load in the subject's blood can be detected by measuring influenza virus antigens (e.g., HA) using an immunoassay such as an ELISA. Viral load can also be detected by amplifying influenza virus nucleic acids (see, e.g., Di Trani et al., BMC Infect. Dis., 6:87 (2006); Ward et al., J. Clin. Virol., 29:179-188 (2004); and Boivin et al., J. Infect. Dis., 188:578-580 (2003)) or by a conventional plaque assay using, e.g., monolayers of Madin-Darby Canine Kidney (MDCK) cells.

### VIII. EXAMPLES

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Selection of Candidate Influenza siRNA

Candidate influenza sequences were identified by scanning influenza nucleocapsid protein (NP) (Genbank Accession No. AY818138) and polymerase (PA) (Genbank Accession No. AY818132) sequences to identify AA dinucleotide motifs and the 19 nucleotides 3′ of the motif. The following candidate sequences were eliminated: (1) sequences comprising a stretch of 4 or more of the same base in a row; (2) sequences comprising homopolymers of Gs; (3) sequences comprising triple base motifs (GGG, CCC, AAA, or TTT); and (4) sequences comprising stretches of 7 or more G/Cs in a row.

Reynold's Rational Design criteria was then applied to the remaining candidate sequences to identify sequences with 5 or more of the following criteria:
1. 30%-52% GC content;
2. At least 3 A/Us at positions 15-19 (sense);
3. Absence of internal repeats;
4. A at position 19 (sense);
5. A at position 3 (sense);
6. U at position 10 (sense);
7. No G/C at position 19 (sense); and
8. No G at position 13 (sense).

Only results with a score of 6 or more in the Stockholm rules (see, Chalk, Wahlestedt, and Sonnhammer method described in Chalk et al., Biochem. Biophys. Res. Commun., 319:264-274 (2004)) were retained.

Next, sequences with a high score from, e.g., Classification tree method or Chalk, Wahlestedt, and Sonnhammer method, were retained.

Next, sequences with a score of 3 or more based on the rules of Amarzguioui and Prydz, Biochem. Biophys. Res. Commun., 316:1050-1058 (2004), were retained.

Next, sequences with thermodynamics >0 were eliminated.

Finally, BLASTn was used to compare the sequences with the mouse and human databases and sequences with homology to ≧15-16 contiguous bp from the center of the target sequence (bp 3-18) against any relevant gene were eliminated. The candidate sequences are shown in Tables 1 and 2.

TABLE 1

siRNA sequences that target Influenza
A virus NP expression.

| | | | | |
|---|---|---|---|---|
| | | SEQ | | SEQ |
| Start | Sense Strand | ID | Antisense Strand | ID |
| Position | (5′→3′) | NO. | (5′→3′) | NO. |

| | | | | |
|---|---|---|---|---|
| 381 | GGACGCAACUGCUGGUCUU | 6 | AAGACCAGCAGUUGCGUCC | 7 |
| 417 | GCAUUCCAAUCUAAAUGAU | 8 | AUCAUUUAGAUUGGAAUGC | 9 |
| 606 | CGACCGGAAUUUCUGGAGA | 10 | UCUCCAGAAAUUCCGGUCG | 11 |
| 641 | GAACAAGGAUUGCAUAUGA | 12 | UCAUAUGCAAUCCUUGUUC | 13 |
| 926 | ACAGCCAGGUCUUUAGUCU | 14 | AGACUAAAGACCUGGCUGU | 15 |
| 1014 | UGAGGACCUUAGAGUCUCA | 16 | UGAGACUCUAAGGUCCUCA | 17 |
| 1244 | AGAGAAACCUUCCCUUCGA | 18 | UCGAAGGGAAGGUUUCUCU | 19 |
| 1268 | CGACCAUUAUGGCAGCAUU | 20 | AAUGCUGCCAUAAUGGUCG | 21 |
| 1322 | GGACUGAAAUCAUAAGAAU | 22 | AUUCUUAUGAUUUCAGUCC | 23 |
| 1437 | UGACAUGAAUAAUGAAGGA | 24 | UCCUUCAUUAUUCAUGUCA | 25 |

The sense and/or antisense strand may contain “dTdT” or “UU” 3′ overhangs.

TABLE 2

siRNA sequences that target Influenza
A virus PA expression.

| | | SEQ | | SEQ |
|---|---|---|---|---|
| Start | Sense Strand | ID | Antisense Strand | ID |
| Position | (5′→3′) | NO. | (5′→3′) | NO. |
| 95 | CGAACAAGUUUGCUGCAAU | 26 | AUUGCAGCAAACUUGUUCG | 27 |
| 165 | UGAACGGAGUGAAUCAAUA | 28 | UAUUGAUUCACUCCGUUCA | 29 |
| 203 | CGAAUGCAUUAUUGAAACA | 30 | UGUUUCAAUAAUGCAUUCG | 31 |
| 306 | ACCUAAAUUUCUCGCAGAU | 32 | AUCUGGGAGAAAUUUAGGU | 33 |
| 308 | CUAAAUUUCUCCCAGAUUU | 34 | AAAUCUGGGAGAAAUUUAG | 35 |
| 340 | GAGAACCGAUUCAUCGAAA | 36 | UUUCGAUGAAUCGGUUCUC | 37 |
| 341 | AGAACCGAUUCAUCGAAAU | 38 | AUUUCGAUGAAUCGGUUCU | 39 |
| 371 | GGAGGGAAGUUCAUACAUA | 40 | UAUGUAUGAAGUUCCCUCC | 41 |
| 753 | AGAAGUGAAUGCUAGAAUU | 42 | AAUUCUAGCAUUCACUUCU | 43 |
| 919 | GCAAUCAAAUGCAUGAAGA | 44 | UCUUCAUGCAUUUGAUUGC | 45 |
| 923 | UCAAAUGCAUGAAGACAUU | 46 | AAUGUCUUCAUGCAUUUGA | 47 |
| 1431 | GGAUGACUUUCAACUGAUU | 48 | AAUCAGUUGAAAGUCAUCC | 49 |
| 1440 | UCAACUGAUUCCAAUGAUA | 50 | UAUCAUUGGAAUCAGUUGA | 51 |
| 1569 | GGAAUUCUCUCUUACUGAU | 52 | AUCAGUAAGAGAGAAUUCC | 53 |

The sense and/or antisense strand may contain “dTdT” or “UU” 3′ overhangs.

### Example 2

### In Vitro Knockdown of Influenza Virus Using siRNA Lipoplexes

This example illustrates that siRNA lipoplexes targeting influenza nucleocapsid protein (NP) or polymerase (PA) sequences can significantly reduce the cytopathic effect of influenza virus and provide substantial viral knockdown in a mammalian cell line.

The influenza virus (e.g., Influenza A H1N1) produces a cytopathic effect (CPE) in Madin-Darby Canine Kidney (MDCK) cells upon infection in the presence of trypsin. The in vitro influenza infection was performed according to the following protocol:
1. MDCK cells were seeded in 96 well plates at about 8000 cells/well (about 8×10⁴ cells/ml) so that the cells were at about 50% density 24 hours after seeding.
2. About 24 hours later, media was changed to fresh complete media (no antibiotics) and cells were transfected with nucleic acid (e.g., siRNA) in Lipofectamine™ 2000 (LF2000).
3. About 4 hours later, complexes were removed, cells were washed with PBS, and cells were infected with various dilutions of influenza virus (e.g., Influenza A H1N1) in virus infection media (DMEM, 0.3% BSA, 10 mM HEPES), adding about 50 μl diluted virus/well.
4. Virus was incubated on cells for about 1-2 hours at 37° C., followed by removal of virus and addition of about 200 μl of virus growth media (DMEM, 0.3% BSA, 10 mM HEPES, 0.25 μg/ml trypsin).
5. Cells were monitored for cytopathic effect at about 48 hours.
6. Influenza HA enzyme immunoassays (EIA) were performed on supernatants.

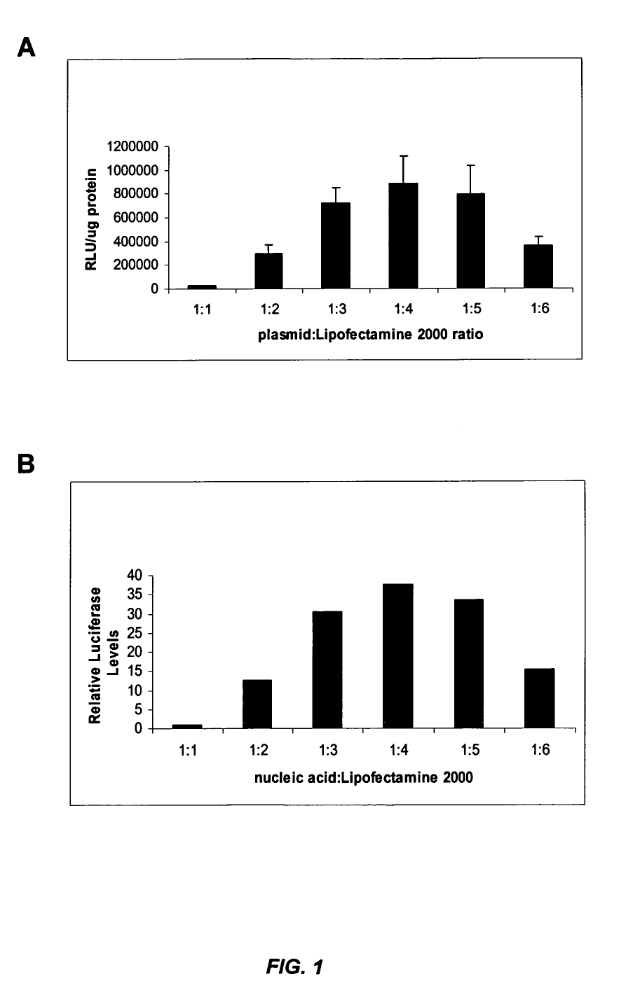

MDCK cells were transfected with a luciferase plasmid and an increasing amount of LF2000 to determine the optimal plasmid:LF2000 ratio for transfection. As shown in FIG. 1, the highest level of luciferase activity was observed with 1 μg plasmid:4 μl LF2000 (i.e., a 1:4 plasmid:LF2000 ratio). The addition of the complexes to MDCK cells at 50% cell density for 4 hours followed by media change did not induce toxicity at any of the concentrations of LF2000.

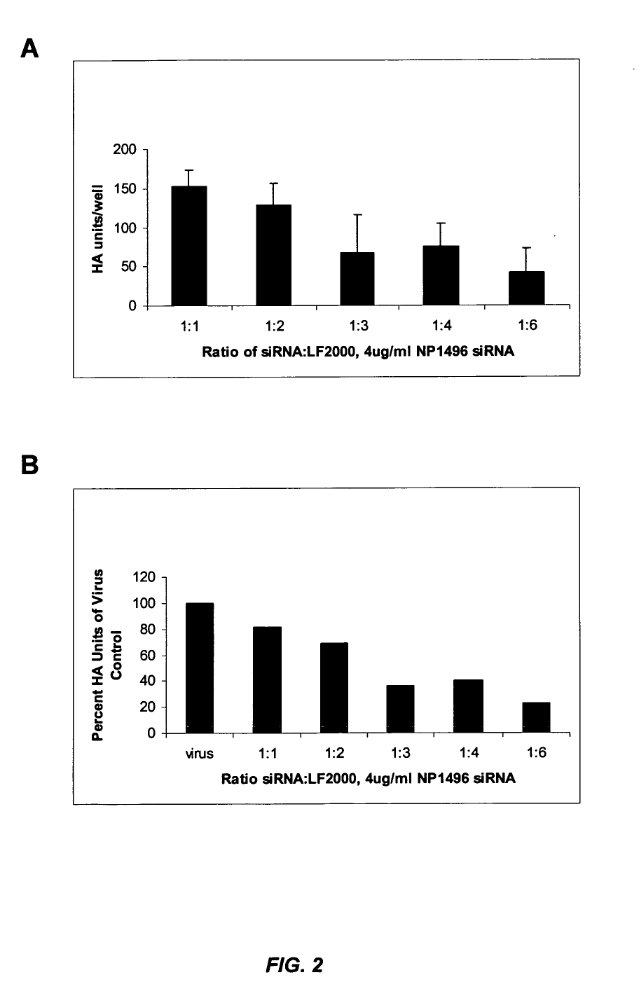

To determine the optimal siRNA:LF2000 ratio for knocking down viral infection, MDCK cells were transfected with an siRNA targeting the nucleocapsid protein (NP 1496) and an increasing amount of LF2000 and then infected with influenza virus. As shown in FIG. 2, the best knockdown of influenza virus occurred at a 1:6 ratio of siRNA:LF2000. However, taking into consideration the optimal plasmid:LF2000 ratio for luciferase, a 1:4 ratio of siRNA:LF2000 was chosen for testing a panel of anti-flu siRNA sequences.

Using the above protocol, a panel of siRNA sequences targeting influenza nucleocapsid protein (NP) or polymerase (PA) sequences was tested for the ability to significantly reduce the cytopathic effect (CPE) produced by the influenza virus at about 48 hours after infection. As used herein, the term “cytopathic effect” or “CPE” refers to a cytopathological evident during viral infection that ultimately leads to cell death. The siRNA sequences were also tested for the amount of HA produced (i.e., HA units/well) and the percentage of HA produced relative to a virus only control (i.e., percent knockdown). The NP siRNA sequences used in this study are provided in Table 3. The PA and control siRNA sequences used in this study are provided in Table 4.

TABLE 3

NP siRNA sequences used in the in vitro
influenza knockdown assay.

| | |
|---|---|
| Name | NP siRNA Sequence |

| | | |
|---|---|---|
| NP 180 | 5′-CGAACUCAAACUCAGUGAUdTdT-3′ | (SEQ ID NO:54) |
| | 3′-dTdTGCUUGAGUUUGAGUCACUA-5′ | (SEQ ID NO:55) |
| NP 952 | 5′-CCUUUCAGACUGCUUCAAAdTdT-3′ | (SEQ ID NO:56) |
| | 3′-dTdTGGAAAGUCUGACGAAGUUU-5′ | (SEQ ID NO:57) |
| NP 411 | 5′-AGCUAAUAAUGGUGACGAUdTdT-3′ | (SEQ ID NO:58) |
| | 3′-dTdTUCGAUUAUUACCACUGCUA-5′ | (SEQ ID NO:59) |
| NP 604 | 5′-GGAACAAUGGUGAUGGAAUdTdT-3′ | (SEQ ID NO:60) |
| | 3′-dTdTCCUUGUUACCACUACCUUA-5′ | (SEQ ID NO:61) |
| NP 929 | 5′-GAUACUCUCUAGUCGGAAUdTdT-3′ | (SEQ ID NO:62) |
| | 3′-dTdTCUAUGAGAGAUCAGCCUUA-5′ | (SEQ ID NO:63) |
| NP 1116 | 5′-GCUUUCCACUAGAGGAGUUdTdT-3′ | (SEQ ID NO:64) |
| | 3′-dTdTCGAAAGGUGAUCUCCUCAA-5′ | (SEQ ID NO:65) |
| NP 1496 | 5′-GGAUCUUAUUUCUUCGGAGdTdT-3′ | (SEQ ID NO:66) |
| | 3′-dTdTCCUAGAAUAAAGAAGCCUC-5′ | (SEQ ID NO:67) |

Column 1: The number refers to the nucleotide position of the 5′ base of the sense strand relative to the Influenza A virus NP ssRNA sequence NC_004522.

TABLE 4

PA siRNA sequences used in the in vitro
influenza knockdown assay.

| | |
|---|---|
| Name | PA siRNA Sequence |

| | | |
|---|---|---|
| PA 626 | 5′-CACAGAGAACAAUAGGUAAdTdT-3′ | (SEQ ID NO:68) |
| | 3′-dTdTGUGUCUCUUGUUAUCCAUU-5′ | (SEQ ID NO:69) |
| PA 848 | 5′-GCAAUGAGAAGAAAGCAAAdTdT-3′ | (SEQ ID NO:70) |
| | 3′-dTdTCGUUACUCUUCUUUCGUUU-5′ | (SEQ ID NO:71) |
| PA 1467 | 5′-GUCUUACAUAAACAGAACAdTdT-3′ | (SEQ ID NO:72) |
| | 3′-dTdTCAGAAUGUAUUUGUCUUGU-5′ | (SEQ ID NO:73) |
| PA 1898 | 5′-GCAACCCACUGAACCCAUUdTdT-3′ | (SEQ ID NO:74) |
| | 3′-dTdTCGUUGGGUGACUUGGGUAA-5′ | (SEQ ID NO:75) |
| PA 2256 | 5′-GAAGAUCUGUUCCACCAUUdTdT-3′ | (SEQ ID NO:76) |
| | 3′-dTdTCUUCUAGACAAGGUGGUAA-5′ | (SEQ ID NO:77) |
| PA 2087 | 5′-GCAAUUGAGGAGUGCCUGAdTdT-3′ | (SEQ ID NO:78) |
| | 3′-dTdTCGUUAACUCCUCACGGACU-5′ | (SEQ ID NO:79) |

Column 1: The number refers to the nucleotide position of the 5′ base of the sense strand relative to the Influenza A virus PA ssRNA sequence AF389117.

TABLE 5

Control siRNA sequences used in the
in vitro influenza knockdown assay.

| | |
|---|---|
| Name | Control siRNA Sequence |

| | | |
|---|---|---|
| ApoB | 5′-GUCAUCACACUGAAUACCAAU-3′ | (SEQ ID NO:80) |
| | 3′-CACAGUAGUGUGACUUAUGGUUA-5′ | (SEQ ID NO:81) |
| Luciferase | 5′-GAUUAUGUCCGGUUAUGUAUU-3′ | (SEQ ID NO:82) |
| | 3′-UUCUAAUACAGGCCAAUACAU-5′ | (SEQ ID NO:83) |
| Luciferase | 5′-AUGUAUUGGCCUGUAUUAGUU-3′ | (SEQ ID NO:84) |
| Scrambled | 3′-UUUACAUAACCGGACAUAAUC-5′ | (SEQ ID NO:85) |

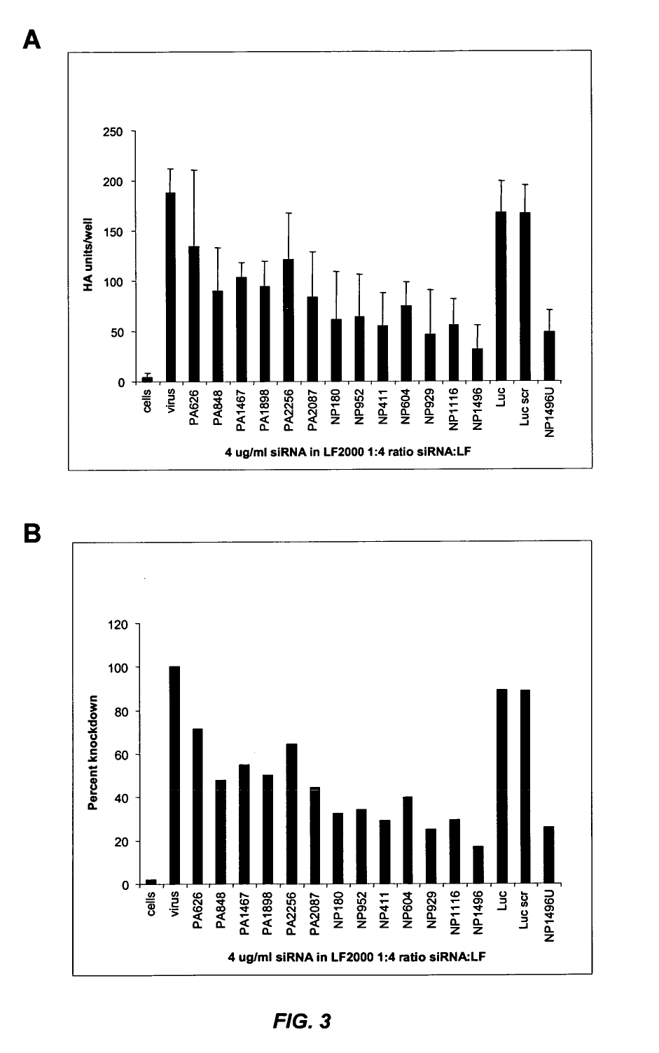

Four siRNA sequences targeting the nucleocapsid protein (i.e., NP 411, NP 929, NP 1116, and NP 1496) provided a significant reduction in CPE and a substantial knockdown of the influenza virus in vitro (see, Table 6 and FIG. 3). For example, NP 1496 provided an 80% reduction in CPE and an 84% knockdown of the influenza virus relative to a virus only control. In contrast, none of the control siRNA sequences (e.g., Luc and Luc scr (i.e., a scrambled luciferase control sequence)) reduced CPE or provided knockdown of the influenza virus.

TABLE 6
Anti-flu siRNA reduces the cytopathic effect
of viral infection in MDCK cells.

| | % CPE (T = 48 h); 5 wells |
|---|---|
| Cells + LF2000 | 5 |
| Virus + LF2000 | 90 |
| PA 626 | 4/5: 90; 1/5: 5 |
| PA 848 | 4/5: 80; 1/5: 60 |
| PA 1467 | 90 |
| PA 1898 | 90 |
| PA 2256 | 4/5: 90; 1/5: 60 |
| PA 2087 | 4/5: 90; 1/5: 30 |
| NP 180 | 2/5: 80; 2/5: 40; 1/5: 10 |
| NP 952 | 1/5: 90; 3/5: 75; 1/5: 10 |
| NP 411 | 30 |
| NP 604 | 4/5: 80; 1/5: 50 |
| NP 929 | 3/5: 50; 2/5: 10 |
| NP 1116 | 20 |
| NP 1496 | 10 |
| Luc | 90 |
| Luc scr (scrambled) | 90 |

This study demonstrates that anti-flu siRNA lipoplexes containing, e.g., NP or PA siRNA, can significantly reduce the cytopathic effect of influenza virus and provide substantial viral knockdown in vitro.

### Example 3

### Design of Anti-Influenza siRNA with Selective Chemical Modifications

This example illustrates that minimal 2′OMe modifications at selective positions in siRNA targeting Influenza A NP and PA are sufficient to decrease the immunostimulatory properties of the siRNA while retaining RNAi activity. In particular, selective 2′OMe-uridine modifications in the sense strand of the siRNA duplex provide NP and PA siRNA with a desirable combination of silencing and non-immunostimulatory properties.

Results

Selective modifications to NP and PA siRNA retain viral knockdown activity. A panel of 2′OMe-modified NP and PA siRNA was prepared and their RNAi activity evaluated in Madin-Darby Canine Kidney (MDCK) cells. The NP siRNA duplexes used in this study are provided in Table 7. The PA siRNA duplexes used in this study are provided in Table 8. The modifications involved introducing 2′OMe-uridine at selected positions in the sense strand of the NP or PA siRNA sequence, in which the siRNA duplex contained less than about 20% 2′OMe-modified nucleotides. The NP and PA siRNA molecules were formulated as lipoplexes and tested for their ability to significantly reduce the cytopathic effect (CPE) produced by influenza virus at about 48 hours after infection. In particular, the NP and PA siRNA molecules were tested for their ability to reduce the amount of HA produced by influenza virus (i.e., HA units/well). In certain instances, the percentage of HA produced relative to a virus only control (i.e., percent knockdown) was also determined.

TABLE 7
siRNA duplexes comprising sense and antisense NP RNA polynucleotides.

| | | | | |
|---|---|---|---|---|
| | | | % 2′OMe- | % Modified |
| Pos. | Mod. | NP siRNA Sequence | Modified | DS Region |

| 97 | 0/0 | 5′-ACGCCAGAAUGCCACUGAAUU-3′ | (SEQ ID NO:86) | 0/42 = 0% | 0/38 = 0% |
|---|---|---|---|---|---|
| | | 3′-UUUGCGGUCUUACGGUGACUU-5′ | (SEQ ID NO:87) | | |
| 97 | U2/0 | 5′-ACGCCAGAAUGCCACUGAAdTdT-3′ | (SEQ ID NO:88) | 2/42 = 4.8% | 2/38 = 5.3% |
| | | 3′-dTdTUGCGGUCUUACGGUGACUU-5′ | (SEQ ID NO:89) | | |
| 165 | 0/0 | 5′-UCCAAAUGUGCACAGAACUUU-3′ | (SEQ ID NO:90) | 0/42 = 0% | 0/38 = 0% |
| | | 3′-UUAGGUUUACACGUGUCUUGA-5′ | (SEQ ID NO:91) | | |
| 165 | U4/O | 5′-UCCAAAUGUGCACAGAACUdTdT-3′ | (SEQ ID NO:92) | 4/42 = 9.5% | 4/38 = 10.5% |
| | | 3′-dTdTAGGUUUACACGUGUCUUGA-5′ | (SEQ ID NO:93) | | |
| 171 | 0/0 | 5′-UGUGCACAGAACUUAAACUUU-3′ | (SEQ ID NO:94) | 0/42 = 0% | 0/38 = 0% |
| | | 3′-UUACACGUGUCUUGAAUUUGA-5′ | (SEQ ID NO:95) | | |
| 171 | U5/0 | 5′-UGUGCACAGAACUUAAACUdTdT-3′ | (SEQ ID NO:96) | 5/42 = 11.9% | 5/38 = 13.2% |
| | | 3′-dTdTACACGUGUCUUGAAUUUGA-5′ | (SEQ ID NO:97) | | |
| 222 | 0/0 | 5′-GCUUAACAAUAGAGAGAAUUU-3′ | (SEQ ID NO:98) | 0/42 = 0% | 0/38 = 0% |
| | | 3′-UUCGAAUUGUUAUCUCUCUUA-5′ | (SEQ ID NO:99) | | |
| 222 | U4/0 | 5′-GCUUAACAAUAGAGAGAAUdTdT-3′ | (SEQ ID NO:100) | 4/42 = 9.5% | 4/38 = 10.5% |
| | | 3′-dTdTCGAAUUGUUAUCUCUCUUA-5′ | (SEQ ID NO:101) | | |
| 383 | 0/0 | 5′-GAAGAAAUAAGGCGAAUCUUU-3′ | (SEQ ID NO:102) | 0/42 = 0% | 0/38 = 0% |
| | | 3′-UUCUUCUUUAUUCCGCUUAGA-5′ | (SEQ ID NO:103) | | |
| 383 | U3/0 | 5′-GAAGAAAUAAGGCGAAUCUdTdT-3′ | (SEQ ID NO:104) | 3/4 = 7.1% | 3/38 = 7.9% |
| | | 3′-dTdTCUUCUUUAUUCCGCUUAGA-5′ | (SEQ ID NO:105) | | |
| 411 | 0/0 | 5′-AGCUAAUAAUGGUGACGAUdTdT-3′ | (SEQ ID NO:58) | 0/4 = 0% | 0/38 = 0% |
| | | 3′-dTdTUCGAUUAUUACCACUGCUA-5′ | (SEQ ID NO:59) | | |
| 411 | U5/0 | 5′-AGCUAAUAAUGGUGACGAUdTdT-3′ | (SEQ ID NO:106) | 5/42 = 11.9% | 5/38 = 13.2% |
| | | 3′-dTdTUCGAUUAUUACCACUGCUA-5′ | (SEQ ID NO:107) | | |
| 724 | 0/0 | 5′-AGGGAAAUUUCAAACUGCUUU-3′ | (SEQ ID NO:108) | 0/42 = 0% | 0/38 = 0% |
| | | 3′-UUUCCCUUUAAAGUUUGACGA-5′ | (SEQ ID NO:109) | | |
| 724 | U5/0 | 5′-AGGGAAAUUUCAAACUGCUdTdT-3′ | (SEQ ID NO:110) | 5/42 = 11.9% | 5/38 = 13.2% |
| | | 3′-dTdTUCCCUUUAAAGUUUGACGA-5′ | (SEQ ID NO:111) | | |
| 929 | 0/0 | 5′-GAUACUCUCUAGUCGGAAUdTdT-3′ | (SEQ ID NO:62) | 0/42 = 0% | 0/38 = 0% |
| | | 3′-dTdTCUAUGAGAGAUCAGCCUUA-5′ | (SEQ ID NO:63) | | |
| 929 | U6/0 | 5′-GAUACUCUCUAGUCGGAAUdTdT-3′ | (SEQ ID NO:112) | 6/42 = 14.3% | 6/38 = 15.8% |
| | | 3′-dTdTCUAUGAGAGAUCAGCCUUA-5′ | (SEQ ID NO:113) | | |
| 1000 | 0/0 | 5′-UGAGAAUCCAGCACACAAGUU-3′ | (SEQ ID NO:114) | 0/42 = 0% | 0/38 = 0% |
| | | 3′-UUACUCUUAGGUCGUGUGUUC-5′ | (SEQ ID NO:115) | | |
| 1000 | U2/0 | 5′-UGAGAAUCCAGCACACAAGdTdT-3′ | (SEQ ID NO:116) | 2/42 = 4.8% | 2/38 = 5.3% |
| | | 3′-dTdTACUCUUAGGUCGUGUGUUC-5′ | (SEQ ID NO:117) | | |
| 1096 | 0/0 | 5′-GGUGGUCCCAAGAGGGAAGUU-3′ | (SEQ ID NO:118) | 0/42 = 0% | 0/38 = 0% |
| | | 3′-UUCCACCAGGGUUCUCCCUUC-5′ | (SEQ ID NO:119) | | |
| 1096 | U2/0 | 5′-GGUGGUCCCAAGAGGGAAGdTdT-3′ | (SEQ ID NO:120) | 2/42 = 4.8% | 2/38 = 5.3% |
| | | 3′-dTdTCCACCAGGGUUCUCCCUUC-5′ | (SEQ ID NO:121) | | |
| 1116 | 0/0 | 5′-GCUUUCCACUAGAGGAGUUdTdT-3′ | (SEQ ID NO:64) | 0/42 = 0% | 0/38 = 0% |
| | | 3′-dTdTCGAAAGGUGAUCUCCUCAA-5′ | (SEQ ID NO:65) | | |
| 1116 | U5/0 | 5′-GCUUUCCACUAGAGGAGUUdTdT-3′ | (SEQ ID NO:122) | 5/42 = 11.9% | 5/38 = 13.2% |
| | | 3′-dTdTCGAAAGGUGAUCUCCUCAA-5′ | (SEQ ID NO:123) | | |
| 1320 | 0/0 | 5′-UGGCAGCAUUCACUGGGAAUU-3′ | (SEQ ID NO:124) | 0/42 = 0% | 0/38 = 0% |
| | | 3′-UUACCGUCGUAAGUGACCCUU-5′ | (SEQ ID NO:125) | | |
| 1320 | U4/0 | 5′-UGGCAGCAUUCACUGGGAAdTdT-3′ | (SEQ ID NO:126) | 4/42 = 9.5% | 4/38 = 10.5% |
| | | 3′-dTdTACCGUCGUAAGUGACCCUU-5′ | (SEQ ID NO:127) | | |
| 1485 | 0/0 | 5′-UGAGUAAUGAAGGAUCUUAUU-3′ | (SEQ ID NO:128) | 0/42 = 0% | 0/38 = 0% |
| | | 3′-UUACUCAUUACUUCCUAGAAU-5′ | (SEQ ID NO:129) | | |
| 1485 | U6/0 | 5′-UGAGUAAUGAAGGAUCUUAdTdT-3′ | (SEQ ID NO:130) | 6/42 = 14.3% | 6/38 = 15.8% |
| | | 3′-dTdTACUCAUUACUUCCUAGAAU-5′ | (SEQ ID NO:131) | | |
| 1496 | 0/0 | 5′-GGAUCUUAUUUCUUCGGAGdTdT-3′ | (SEQ ID NO:66) | 0/42 = 0% | 0/38 = 0% |
| | | 3′-dTdTCCUAGAAUAAAGAAGCCUC-5′ | (SEQ ID NO:67) | | |
| 1496 | U4/0 | 5′-GGAUCUUAUUUCUUCGGAGdTdT-3′ | (SEQ ID NO:132) | 4/42 = 9.5% | 4/38 = 10.5% |
| | | 3′-dTdTCCUAGAAUAAAGAAGCCUC-5′ | (SEQ ID NO:133) | | |
| 1496 | U8/0 | 5′-GGAUCUUAUUUCUUCGGAGdTdT-3′ | (SEQ ID NO:134) | 8/42 = 19% | 8/38 = 21% |
| | | 3′-dTdTCCUAGAAUAAAGAAGCCUC-5′ | (SEQ ID NO:135) | | |
| Column 1: The number refers to the nucleotide position of the 5′ base of the sense strand relative to the Influenza A virus NP ssRNA sequence NC_004522. Column 2: The numbers refer to the distribution of 2′OMe chemical modifications in each strand. For example, “U5/0” indicates 5 uridine 2′OMe modifications in the sense strand and no uridine 2′OMe modifications in the antisense strand. Column 3: 2′OMe-modified nucleotides are indicated in bold and underlined; | | | | | |
| #“dT” = deoxythymidine. Column 4: The number and percentage of 2′OMe-modified nucleotides in the siRNA duplex are provided. Column 5: The number and percentage of modified nucleotides in the double-stranded (DS) region of the siRNA duplex are provided. | | | | | |

TABLE 8
siRNA duplexes comprising sense and antisense PA RNA polynucleotides.

| | | | | |
|---|---|---|---|---|
| | | | % 2′OMe- | % Modified |
| Pos. | Mod. | PA siRNA Sequence | Modified | DS Region |

| 194 | 0/0 | 5′-GGCGAGUCAAUAAUCGUAGdTdT-3′ | (SEQ ID NO:136) | 0/4 = 0% | 0/38 = 0% |
|---|---|---|---|---|---|
| | | 3′-dTdTCCGCUCAGUUAUUAGCAUC-5′ | (SEQ ID NO:137) | | |
| 194 | U4/0 | 5′-GGCGAGUCAAUAAUCGUAGdTdT-3′ | (SEQ ID NO:138) | 4/42 = 9.5% | 4/38 = 10.5% |
| | | 3′-dTdTCCGCUCAGUUAUUAGCAUC-5′ | (SEQ ID NO:139) | | |
| 212 | 0/0 | 5′-GAACUUGGUGAUCCUAAUGdTdT-3′ | (SEQ ID NO:140) | 0/42 = 0% | 0/38 = 0% |
| | | 3′-dTdTCUUGAACCACUAGGAUUAC-5′ | (SEQ ID NO:141) | | |
| 212 | U6/0 | 5′-GAACUUGGUGAUCCUAAUGdTdT-3′ | (SEQ ID NO:142) | 6/42 = 14.3% | 6/38 = 15.8% |
| | | 3′-dTdTCUUGAACCACUAGGAUUAC-5′ | (SEQ ID NO:143) | | |
| 392 | 0/0 | 5′-AGGAGAGAAGUUCACAUAUdTdT-3′ | (SEQ ID NO:144) | 0/42 = 0% | 0/38 = 0% |
| | | 3′-dTdTUCCUCUCUUCAAGUGUAUA-5′ | (SEQ ID NO:145) | | |
| 392 | U4/0 | 5′-AGGAGAGAAGUUCACAUAUdTdT-3′ | (SEQ ID NO:146) | 4/42 = 9.5% | 4/38 = 10.5% |
| | | 3′-dTdTUCCUCUCUUCAAGUGUAUA-5′ | (SEQ ID NO:147) | | |
| 751 | 0/0 | 5′-GGGCAAGCUGUCUCAAAUGdTdT-3′ | (SEQ ID NO:148) | 0/42 = 0% | 0/38 = 0% |
| | | 3′-dTdTCCCGUUCGACAGAGUUUAC-5′ | (SEQ ID NO:149) | | |
| 751 | U4/0 | 5′-GGGCAAGCUGUCUCAAAUGdTdT-3′ | (SEQ ID NO:150) | 4/42 = 9.5% | 4/38 = 10.5% |
| | | 3′-dTdTCCCGUUCGACAGAGUUUAC-5′ | (SEQ ID NO:151) | | |
| 783 | 0/0 | 5′-AUGCUAGAAUUGAACCUUUdTdT-3′ | (SEQ ID NO:152) | 0/42 = 0% | 0/38 = 0% |
| | | 3′-dTdTUACGAUCUUAACUUGGAAA-5′ | (SEQ ID NO:153) | | |
| 783 | U7/0 | 5′-AUGCUAGAAUUGAACCUUUdTdT-3′ | (SEQ ID NO:154) | 7/42 = 16.7% | 7/38 = 18.4% |
| | | 3′-dTdTUACGAUCUUAACUUGGAAA-5′ | (SEQ ID NO:155) | | |
| 813 | 0/0 | 5′-CACCACGACCACUUAGACUdTdT-3′ | (SEQ ID NO:156) | 0/42 = 0% | 0/38 = 0% |
| | | 3′-dTdTGUGGUGCUGGUGAAUCUGA-5′ | (SEQ ID NO:157) | | |
| 813 | U3/0 | 5′-CACCACGACCACUUAGACUdTdT-3′ | (SEQ ID NO:158) | 3/42 = 7.1% | 3/38 = 7.9% |
| | | 3′-dTdTGUGGUGCUGGUGAAUCUGA-5′ | (SEQ ID NO:159) | | |
| 1656 | 0/0 | 5′-UAGGAGAUAUGCUUCUAAGdTdT-3′ | (SEQ ID NO:160) | 0/42 = 0% | 0/38 = 0% |
| | | 3′-dTdTAUCCUCUAUACGAAGAUUC-5′ | (SEQ ID NO:161) | | |
| 1656 | U6/0 | 5′-UAGGAGAUAUGCUUCUAAGdTdT-3′ | (SEQ ID NO:162) | 6/42 = 14.3% | 6/38 = 15.8% |
| | | 3′-dTdTAUCCUCUAUACGAAGAUUC-5′ | (SEQ ID NO:163) | | |
| 1658 | 0/0 | 5′-GGAGAUAUGCUUCUAAGAAdTdT-3′ | (SEQ ID NO:164) | 0/42 = 0% | 0/38 = 0% |
| | | 3′-dTdTCCUCUAUACGAAGAUUCUU-5′ | (SEQ ID NO:165) | | |
| 1658 | U5/0 | 5′-GGAGAUAUGCUUCUAAGAAdTdT-3′ | (SEQ ID NO:166) | 5/42 = 11.9% | 5/38 = 13.2% |
| | | 3′-dTdTCCUCUAUACGAAGAUUCUU-5′ | (SEQ ID NO:167) | | |
| 1884 | 0/0 | 5′-UUGGAGAGUCUCCCAAAGGdTdT-3′ | (SEQ ID NO:168) | 0/42 = 0% | 0/38 = 0% |
| | | 3′-dTdTAACCUCUCAGAGGGUUUCC-5′ | (SEQ ID NO:169) | | |
| 1884 | U4/0 | 5′-UUGGAGAGUCUCCCAAAGGdTdT-3′ | (SEQ ID NO:170) | 4/42 = 9.5% | 4/38 = 10.5% |
| | | 3′-dTdTAACCUCUCAGAGGGUUUCC-5′ | (SEQ ID NO:171) | | |
| 2098 | 0/0 | 5′-GUGCCUAAUUAAUGAUCCCdTdT-3′ | (SEQ ID NO:172) | 0/42 = 0% | 0/38 = 0% |
| | | 3′-dTdTCACGGAUUAAUUACUAGGG-5′ | (SEQ ID NO:173) | | |
| 2098 | U6/0 | 5′-GUGCCUAAUUAAUGAUCCCdTdT-3′ | (SEQ ID NO:174) | 6/42 = 14.3% | 6/38 = 15.8% |
| | | 3′-dTdTCACGGAUUAAUUACUAGGG-5′ | (SEQ ID NO:175) | | |
| Column 1: The number refers to the nucleotide position of the 5′ base of the sense strand relative to the Influenza A virus PA ssRNA sequence AF389117. Column 2: The numbers refer to the distribution of 2′OMe chemical modifications in each strand. For example, “U5/0” indicates 5 uridine 2′OMe modifications in the sense strand and no uridine 2′OMe modifications in the antisense strand. Column 3: 2′OMe-modified nucleotides are indicated in bold and underlined; | | | | | |
| #“dT” = deoxythymidine. Column 4: The number and percentage of 2′OMe-modified nucleotides in the siRNA duplex are provided. Column 5: The number and percentage of modified nucleotides in the double-stranded (DS) region of the siRNA duplex are provided. | | | | | |

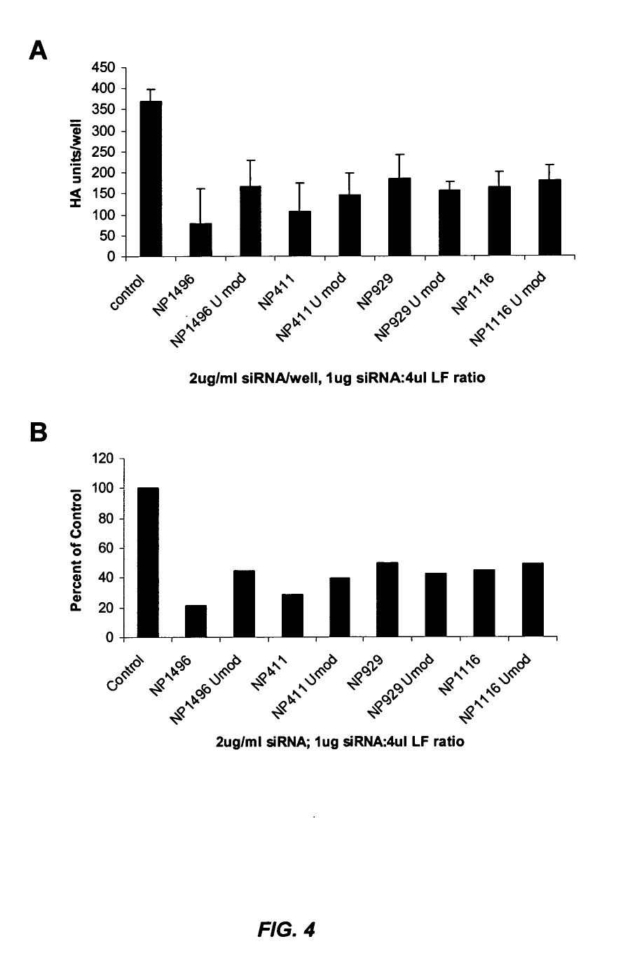

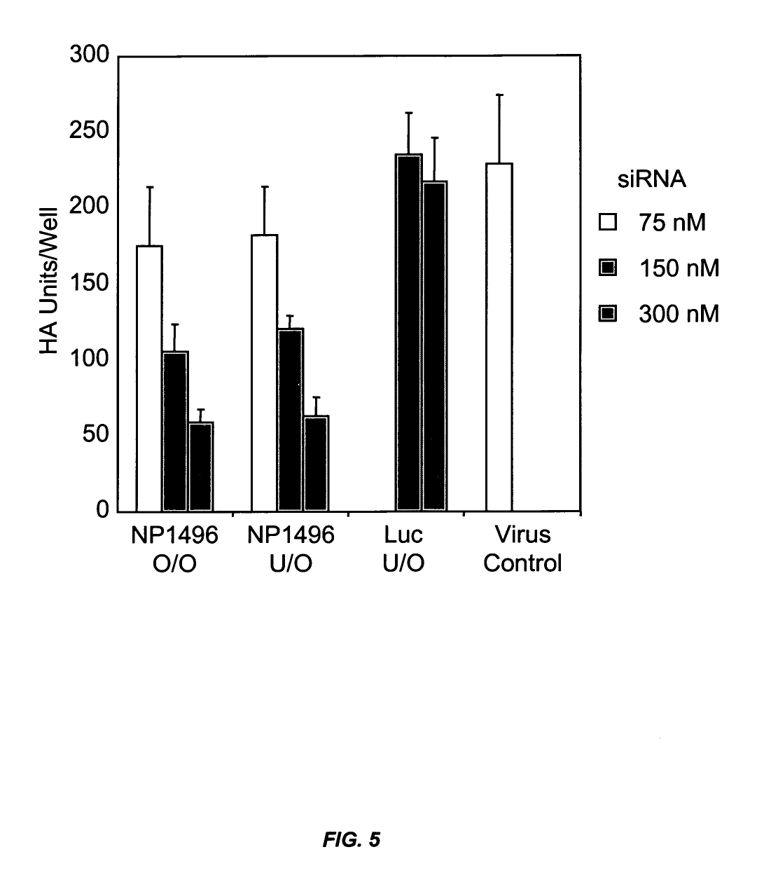

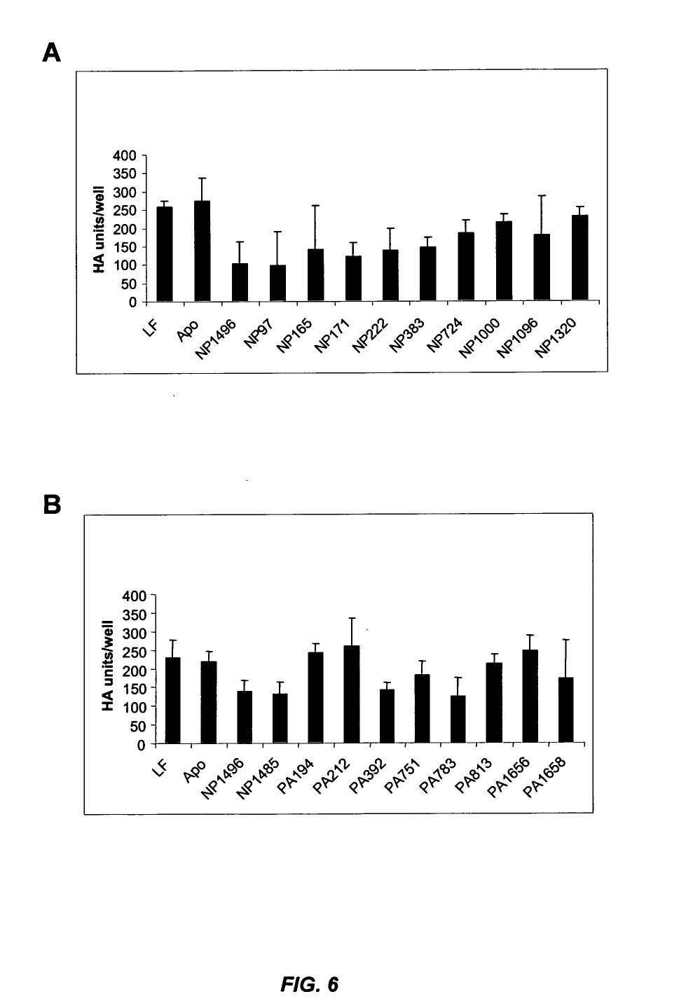

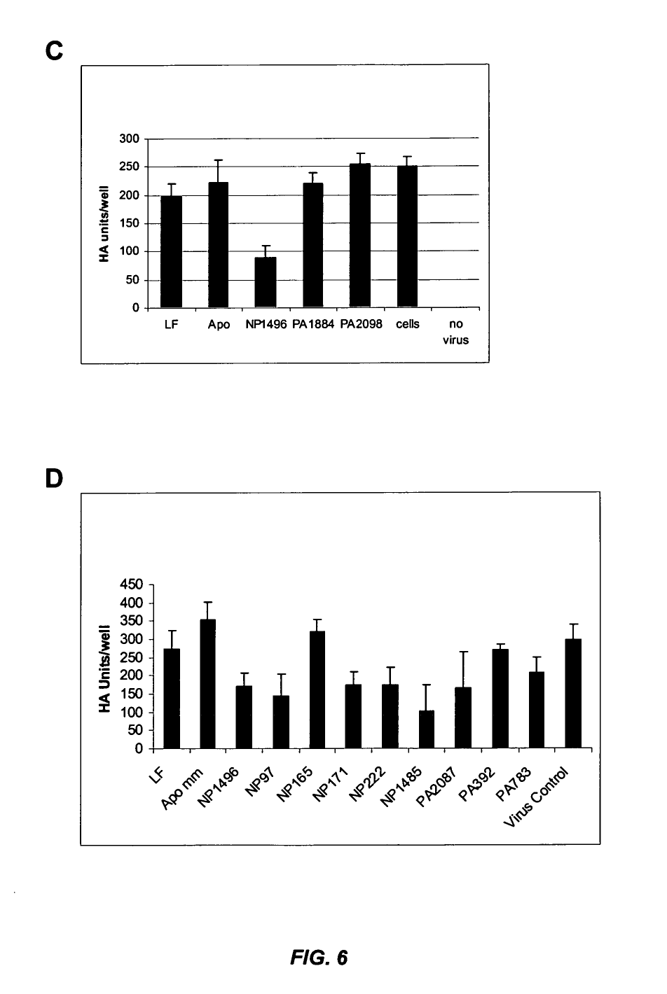

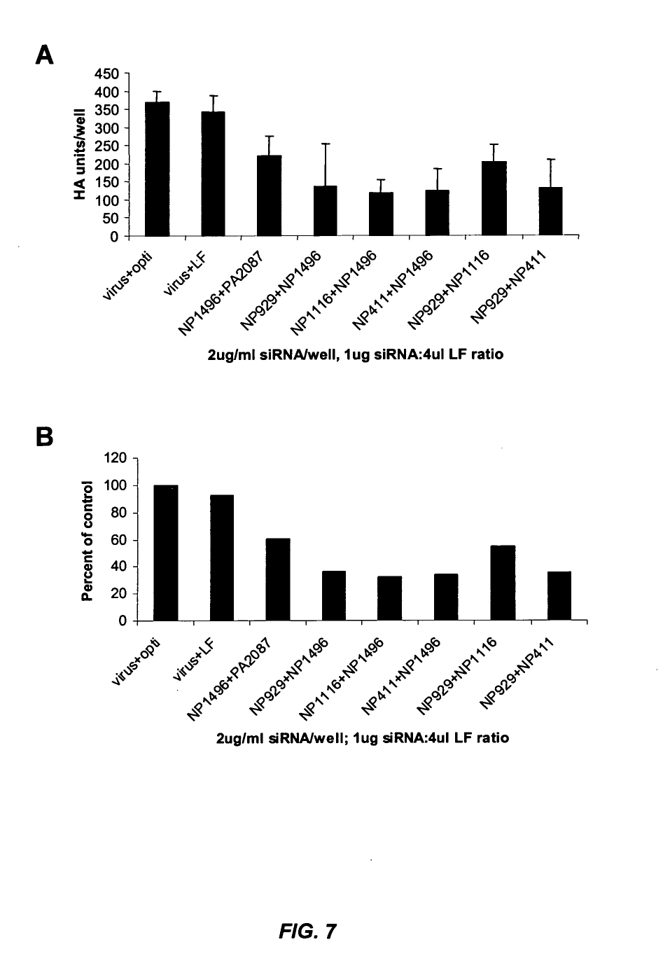

FIGS. 4-6 show that selective 2′OMe modifications to the sense strand of the NP or PA siRNA duplex did not negatively affect influenza knockdown activity when compared to unmodified counterpart sequences or control sequences. FIG. 7 shows that various combinations of these modified NP siRNA molecules provided enhanced knockdown of influenza virus in MDCK cells relative to controls.

These results demonstrate that modified NP 1496, NP 411, NP 929, NP 1116, NP 97, NP 171, NP 222, NP 383, NP 1485, PA 392, and PA 783 siRNA display potent and comparable anti-influenza activity. NP 1485 may be particularly useful against multiple serotypes of the Influenza A virus (e.g., H1N1, H5N1, etc.) because it targets a highly conserved sequence in the NP gene.

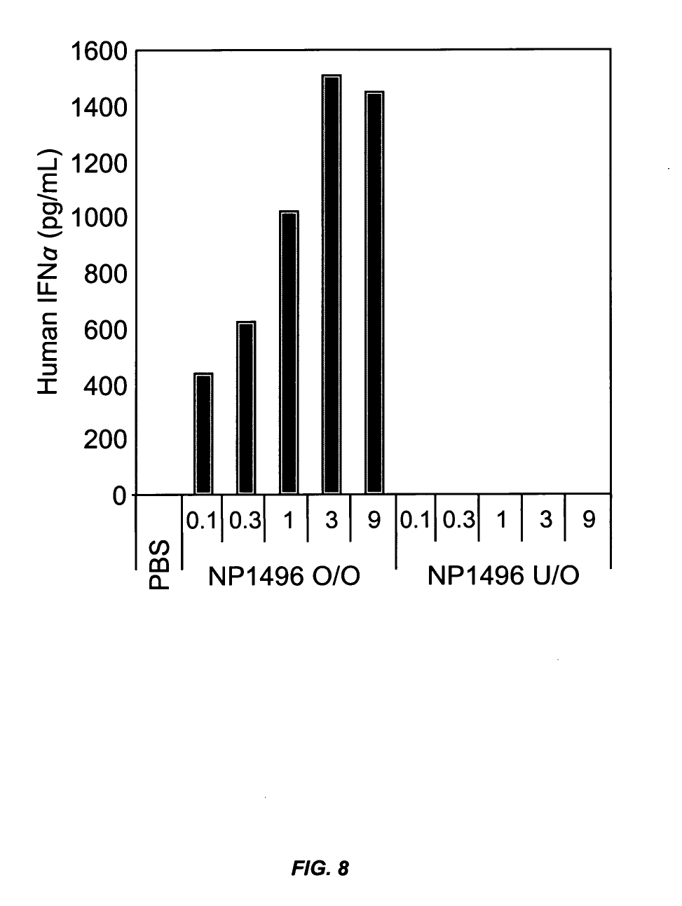

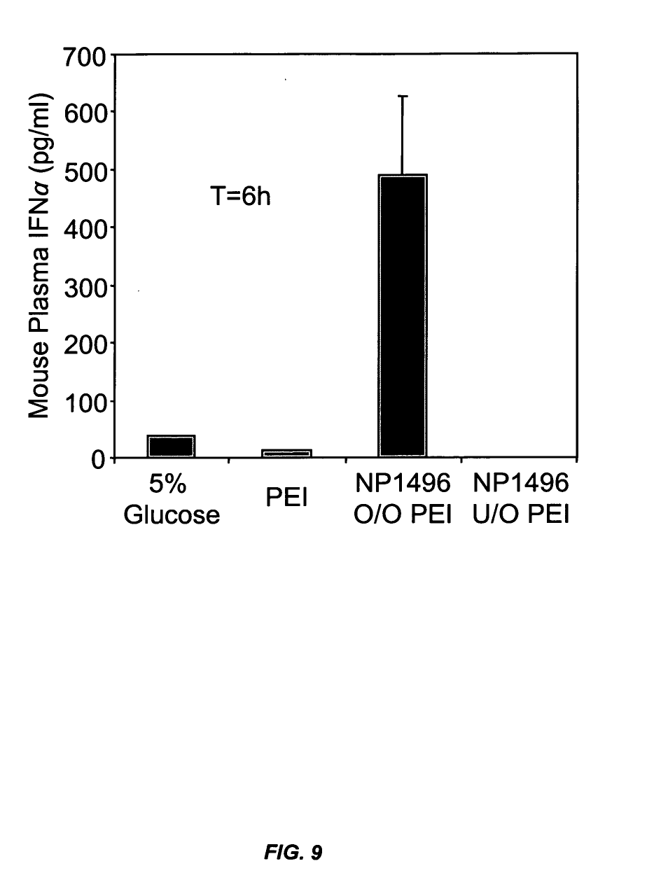

Selective modifications to NP siRNA abrogate in vitro and in vivo cytokine induction. Unmodified NP 1496 siRNA (i.e., 0/0) and a 2′OMe-modified variant thereof (i.e., U8/0) were either encapsulated into SNALPs having 2 mol % PEG-cDMA, 40 mol % DLinDMA, 10 mol % DSPC, and 48 mol % cholesterol or complexed with polyethylenimine (PEI) to form polyplexes. The SNALP-formulated NP-targeting siRNA were tested in vitro to look for the induction of an immune response, e.g., cytokine induction. Human peripheral blood mononuclear cells (PBMCs) were transfected with 40 μg of the SNALP formulation comprising NP 1496 siRNA and supernatants collected for cytokine analysis at 16 hours. The polyplex formulations were tested in vivo to look for the induction of an immune response, e.g., cytokine induction. Mice were intravenously injected with the polyplexes at 120 μg siRNA/mouse and plasma samples were collected 6 hours post-treatment and tested for interferon-α levels by an ELISA assay. FIG. 8 shows that selective 2′OMe modifications to NP 1496 siRNA abrogated interferon induction in an in vitro cell culture system. FIG. 9 shows that selective 2′OMe modifications to NP 1496 siRNA abrogated the interferon induction associated with systemic administration of the native (i.e., unmodified) duplex.

Methods

siRNA: All siRNA used in these studies were chemically synthesized by Protiva Biotherapeutics (Burnaby, BC), University of Calgary (Calgary, AB), or Dharmacon Inc. (Lafayette, Colo.). siRNA were desalted and annealed using standard procedures.

Lipid encapsulation of siRNA: Unless otherwise indicated, siRNAs were encapsulated into liposomes composed of the following lipids; synthetic cholesterol (Sigma; St. Louis, Mo.), the phospholipid DSPC (1,2-distearoyl-sn-glycero-3-phosphocholine; Avanti Polar Lipids; Alabaster, Ala.), the PEG-lipid PEG-cDMA (3-N-[(-Methoxy poly(ethylene glycol)2000)carbamoyl]-1,2-dimyrestyloxy-propylamine), and the cationic lipid DLinDMA (1,2-Dilinoleyloxy-3-(N,N-dimethyl)aminopropane) in the molar ratio 48:10:2:40. In other words, unless otherwise indicated, siRNAs were encapsulated into liposomes of the following SNALP formulation: 2 mol % PEG-cDMA, 40 mol % DLinDMA, 10 mol % DSPC, and 48 mol % cholesterol.

Lipoplex treatment and in vitro influenza infection: The influenza virus (e.g., Influenza A/PR/8/34 H1N1) produces a cytopathic effect in MDCK cells upon infection in the presence of trypsin. The lipoplex treatment and in vitro influenza infection of MDCK cells was performed according to the following protocol:
1. MDCK cells were seeded in 96 well plates at about 8000 cells/well (about 8×10⁴ cells/ml) so that the cells were at about 50% density 24 hours after seeding.
2. About 24 hours later, media was changed to fresh complete media (no antibiotics) and cells were transfected with a lipoplex comprising 2 μg/ml siRNA in Lipofectamine™ 2000 (LF2000) (Invitrogen Corp.; Camarillo, Calif.) at a 1:4 ratio of siRNA:LF2000.
3. About 4 hours later, complexes were removed, cells were washed with PBS, and cells were infected with a 1:800 dilution of influenza virus in virus infection media (DMEM, 0.3% BSA, 10 mM HEPES), adding about 50 μl diluted virus/well.
4. Virus was incubated on cells for about 1-2 hours at 37° C., followed by removal of virus and addition of about 200 μl of virus growth media (DMEM, 0.3% BSA, 10 mM HEPES, 0.25 μg/ml trypsin).
5. Cells were monitored for cytopathic effect at about 48 hours.
6. Influenza HA enzyme immunoassays (EIA) were performed on supernatants.

Polyplex treatment and in vivo cytokine induction: Animal studies were completed in accordance with the Canadian Council on Animal Care guidelines following approval by the local Animal Care and Use Committee at Protiva Biotherapeutics. 6-8 week old CD1 ICR mice (Harlan; Indianapolis, Ind.) were subjected to a three week quarantine and acclimation period prior to use. siRNAs were mixed with In vivo jetPEI™ (Qbiogene, Inc.; Carlsbad, Calif.) according to the manufacturer's instructions at an N/P ratio of 5 at room temperature for 20 min. Mice were administered the In vivo jetPEI™ polyplexes, corresponding to 120 μg siRNA/mouse, by standard intravenous injection in the lateral tail vein in 0.2 ml PBS. Blood was collected by cardiac puncture 6 hours after administration and processed as plasma for cytokine analysis. Interferon-α levels in plasma were measured using a sandwich ELISA method according to the manufacturer's instructions (PBL Biomedical; Piscataway, N.J.). Additional methods for PEI polyplex formation are provided in Judge et al., Nat. Biotechnol., 23:457-462 (2005).

In vitro cytokine induction: PBMCs were transfected with from 0.1 μg/ml to 9 μg/ml of SNALP-formulated siRNA and interferon-α levels were assayed in cell culture supernatants after 16 hours using a sandwich ELISA method according to the manufacturer's instructions (PBL Biomedical; Piscataway, N.J.).

### Example 4

### In Vivo Knockdown of Influenza Virus Using SNALP

This example provides a study investigating the effect of an anti-flu SNALP against the influenza virus in infected mice. Specifically, the study had the following objectives: (1) to evaluate influenza knockdown with siRNA targeting an influenza nucleocapsid protein (NP) sequence (i.e., NP siRNA); (2) to determine a dose response of NP siRNA encapsulated within SNALP; (3) to titer the Influenza A PR/8/34 stock to obtain an appropriate concentration for survival studies; and (4) to investigate high doses of naked NP siRNA as a specific positive control for influenza knockdown.

The synthetic modified siRNA used in this study were obtained from Dharmacon Inc. (Lafayette, Colo.). The siRNA sequences are provided in Table 9.

TABLE 9
Modified siRNA sequences used in the in
vivo influenza knockdown study.

| | |
|---|---|
| Name | 2′OMe-Modified siRNA Sequence |

| | | |
|---|---|---|
| NP 1496 (U4/0) | 5′-GGAUCUUAUUUCUUCGGAGdTdT-3′ | (SEQ ID NO:132) |
| | 3′-dTdTCCUAGAAUAAAGAAGCCUC-5′ | (SEQ ID NO:133) |
| ApoB Mismatch (mm) | 5′-GUGAUCAGACUCAAUACGAAU-3′ | (SEQ ID NO:176) |
| | 3′-CACACUAGUCUGAGUUAUGCUUA-5′ | (SEQ ID NO:177) |

2′OMe-modified nucleotides are indicated in bold and underlined; “dT” = deoxythymidine.

The in vivo knockdown was performed according to the following protocol using 40 female Balb/c mice housed at 4 mice per cage:

Study Timeline:

1. Mice were ordered.
2. Mice arrived.
3. Mice were taken out of quarantine.
4. Mice were treated with SNALP containing 2% PEG-C-DMA, 40% DLindMA, 10% DSPC (2:40:10), and 48% cholesterol at a 1× drug:lipid ratio.
5. Mice were treated with influenza A/PR/8/34 about 4 hours after SNALP pretreatment.
6. Mice were sacrificed.

Experimental Design:

| | | | Infectious | |
|---|---|---|---|---|
| | # | Test Article | Article | Collection/ |
| Group | Mice | (−4 h) | (0 h) | Readout |
| A | 2 | Saline | Saline | 48 h sac |
| B | 6 | PBS | Influenza | 48 h sac |
| | | | A/PR/8/34 | |
| | | | 1:40,000 | |
| C | 4 | Modified ApoB | Influenza | 48 h sac |
| | | mismatch (mm) | A/PR/8/34 | |
| | | 0.5 mg/kg = 0.25 | 1:40,000 | |
| | | mg/ml | | |
| D | 4 | Modified NP 1496 | Influenza | 48 h sac |
| | | 0.25 mg/kg = 0.125 | A/PR/8/34 | |
| | | mg/ml | 1:40,000 | |
| E | 4 | Modified NP 1496 | Influenza | 48 h sac |
| | | 0.5 mg/kg = 0.25 | A/PR/8/34 | |
| | | mg/ml | 1:40,000 | |
| F | 4 | Modified NP 1496 | Influenza | 48 h sac |
| | | 1 mg/kg = 0.5 | A/PR/8/34 | |
| | | mg/ml | 1:40,000 | |
| G | 4 | Naked NP 1496 | Influenza | 48 h sac |
| | | 12.5 mg/kg = 5 | A/PR/8/34 | |
| | | mg/ml | 1:40,000 | |
| H | 6 | 1:60,000 | Influenza | 14 d |
| | | Survival | A/PR/8/34 | survival |
| | | | 1:40,000 | |
| I | 6 | 1:80,000 | Influenza | 14 d |
| | | Survival | A/PR/8/34 | survival |
| | | | 1:40,000 | |

SNALP Preparation:

Sample A: Saline=5 mice×50 μl=250 μl needed

Sample B: PBS=10 mice×50 μl=500 μl needed

Sample C: Modified ApoB mm SNALP (2:40:10) @ 0.25 mg/ml=5 mice×50 μl (0.5mg/kg)=250 μl needed
(1.048 mg/ml)×=(0.25 mg/ml)(0.250 ml)
x=0.060 mls (added 0.190 ml PBS)

Sample D: Modified NP 1496 SNALP (2:40:10) @ 0.125 mg/ml=5 mice×50 μl (0.25 mg/kg)=250 μl needed
(0.998 mg/ml)×=(0.125 mg/ml)(0.250 ml)
x=0.031 mls (added 0.219 ml PBS)

Sample E: Modified NP 1496 SNALP (2:40:10) @ 0.25 mg/ml=5 mice×50 μl (0.5 mg/kg)=250 μl needed
(0.998 mg/ml)×=0.25 mg/ml)(0.250 ml)
x=0.063 mls (added (0.187 ml PBS)

Sample F: Modified NP 1496 SNALP (2:40:10) @ 0.5 mg/ml=5 mice×50 μl (1.0 mg/kg)=250 μl needed
(0.998 mg/ml)x=(0.5 mg/ml)(0.250 ml)
x=0.125 mls (added 0.125 ml PBS)

Sample G: Naked NP 1496 @ 5 mg/ml=5 mice×50 μl (12.5 mg/kg)=250 μl needed
(6 mg/ml)×=(5 mg/ml)(0.250 ml)
x=0.208 ml (added 0.042 ml 30% glucose in water to get final [glucose]=5%
(5%)(0.250 ml)=x(0.042 ml)
x=30%
   Viral Preparation:

30 mice inoculated @ [1:40,000 dilution of virus stock at 2freeze/thaws] in total volume of 50 μl per mouse=30×50 μl=1500 μl
Prepare 3000 μl of 1:40,000 dilution
Prepare 1:100 (10 μl stock in 990 μl saline)
Prepare 1:1,000 (100 μl of 1:100 dilution in 900 μl saline)
Prepare 1:40,000 (75 μl of 1:1000 dilution in 2925 μl saline)

6 mice inoculated @ [1:60,000 dilution of virus stock at 2freeze/thaws] in total volume of 50 μl per mouse=10×50 μl=500 μl
Add 667 μl of 1:40,000 in 333 μl saline

6 mice inoculated @ [1:80,000 dilution of virus stock at 2freeze/thaws] in total volume of 50 μl per mouse=10×50 μl=500 μl
Add 500 μl of 1:40,000 in 500 μl saline
   Treatment:

Mice were treated with a range of concentrations of Influenza A PR/8/34 intranasally in a total volume of 50 μl.

Endpoint:

Viral burden has not been previously investigated and was one of the objectives of this study. Possible signs of distress have been documented in the literature and were used as signs of morbidity and mortality prior to euthanasia. The primary indicator of infection for this model was body weight. When mice reached >20% body weight loss, lungs were harvested and blood was collected into microtainer EDTA tubes via cardiac puncture. Body temperature was another method for detecting grade of infection. Mice exhibiting signs of distress associated with viral treatment were terminated at the discretion of the vivarium staff.

Symptoms of influenza infection should manifest within 10 to 14 days. If this is not the case, a higher viral titer should be examined.

Termination:

Mice were terminated by CO₂ inhalation followed by cervical dislocation.

Data Analysis:

Daily body weight and cage-side observations were performed. Enzyme immunoassays (EIA), e.g., hemagglutinin (HA) EIA, were performed on lung samples.

Results:

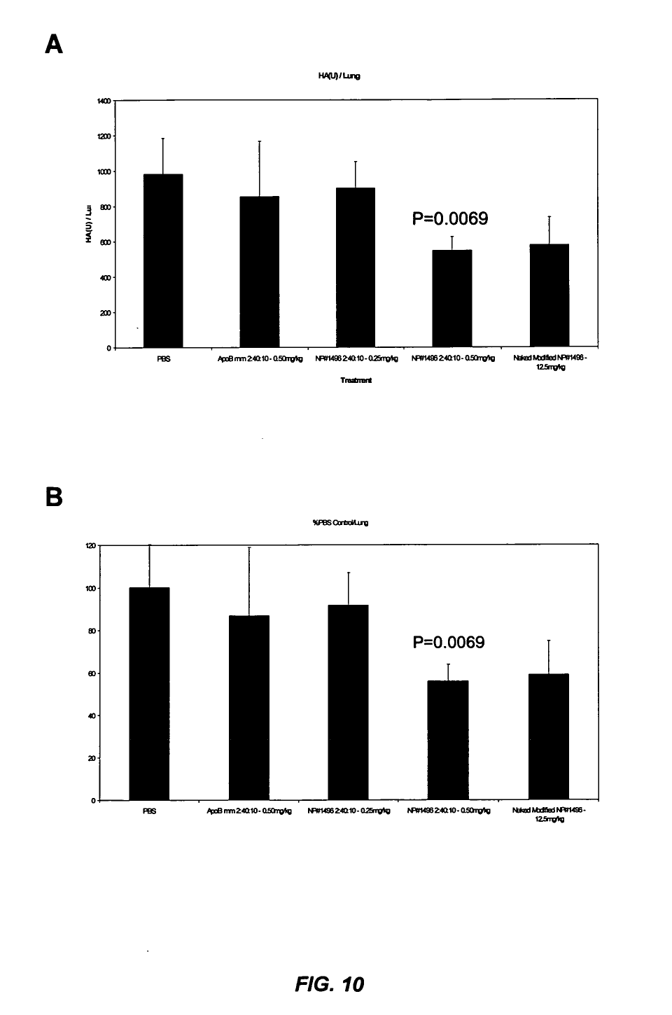

As shown in FIG. 10, pretreatment of mice by intranasally administering SNALP containing 2′OMe-modified NP 1496 siRNA at 0.5 mg/kg about 4 hours prior to Influenza A/PR/8/34 infection had a significant effect on viral infection in vivo. Not only did the amount of HA produced (i.e., HA units/lung) significantly decrease, but the percentage of HA produced relative to a PBS control (i.e., percent knockdown) decreased by over 40% (p=0.0069). The viral knockdown was highly sequence-specific, as a 2′OMe-modified ApoB mismatch (mm) siRNA did not have a significant effect on inhibiting viral infection in vivo. FIG. 10 also shows that naked 2′OMe-modified NP 1496 siRNA at a very high dose (i.e., 12.5 mg/kg) could serve as a specific positive control for influenza knockdown.

This example demonstrates that anti-flu siRNA encapsulated within lipid particles such as SNALPs can provide substantial viral knockdown in mice inoculated with the influenza virus.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents, PCT publications, and Accession Nos. are incorporated herein by reference for all purposes.

What is claimed is:

1. An siRNA molecule comprising a double-stranded region of about 15 to about 60 nucleotides in length, wherein said siRNA molecule silences expression of an influenza virus gene selected from the group consisting of PA, PB1, PB2, NP, M1, M2, NS1, and NS2.

2. The siRNA molecule in accordance with claim 1, wherein said influenza virus is selected from the group consisting of Influenza A, B, and C.

3. The siRNA molecule in accordance with claim 1, wherein said influenza virus gene is selected from the group consisting of NP and PA.

4. The siRNA molecule in accordance with claim 1, wherein said influenza virus gene is NP.

5. The siRNA molecule in accordance with claim 1, wherein said influenza virus gene is PA.

6. The siRNA molecule in accordance with claim 1, wherein said siRNA molecule comprises at least one of the sequences set forth in Tables 1-4 and 7-8.

7. The siRNA molecule in accordance with claim 1, wherein said siRNA molecule comprises at least one of the sequences set forth in Tables 7-8.

8. The siRNA molecule in accordance with claim 1, wherein said siRNA molecule is selected from the group consisting of NP 97, NP 171, NP 222, NP 383, NP 411, NP 929, NP 1116, NP 1485, PA 392, PA 783, and a mixture thereof.

9. The siRNA molecule in accordance with claim 1, wherein said siRNA molecule is NP 1485.

10. The siRNA molecule in accordance with claim 1, wherein said siRNA molecule comprises a double-stranded region of about 15 to about 30 nucleotides in length.

11. The siRNA molecule in accordance with claim 1, wherein said double-stranded region comprises at least one modified nucleotide.

12. The siRNA molecule in accordance with claim 11, wherein said at least one modified nucleotide is selected from the group consisting of a 2′-O-methyl (2′OMe) nucleotide, 2′-deoxy-2′-fluoro (2′F) nucleotide, 2′-deoxy nucleotide, 2′-O-(2-methoxyethyl) (MOE) nucleotide, locked nucleic acid (LNA) nucleotide, and mixtures thereof.

13. The siRNA molecule in accordance with claim 11, wherein said at least one modified nucleotide is a modified uridine nucleotide, modified guanosine nucleotide, or mixtures thereof.

14. The siRNA molecule in accordance with claim 11, wherein all of the uridine nucleotides in one strand of said siRNA molecule comprise modified uridine nucleotides.

15. The siRNA molecule in accordance with claim 14, wherein all of the uridine nucleotides in the sense strand of said siRNA molecule comprise modified uridine nucleotides.

16. The siRNA molecule in accordance with claim 14, further comprising at least one modified nucleotide selected from the group consisting of a modified guanosine nucleotide, modified adenosine nucleotide, modified cytosine nucleotide, and mixtures thereof.

17. The siRNA molecule in accordance with claim 11, wherein said at least one modified nucleotide is a 2′OMe nucleotide.

18. The siRNA molecule in accordance with claim 11, wherein said at least one modified nucleotide is selected from the group consisting of a 2′OMe-guanosine nucleotide, 2′OMe-uridine nucleotide, 2′OMe-adenosine nucleotide, and mixtures thereof.

19. The siRNA molecule in accordance with claim 11, wherein said at least one modified nucleotide is not a 2′OMe-cytosine nucleotide.

20. The siRNA molecule in accordance with claim 11, wherein said at least one modified nucleotide is a 2′OMe-uridine nucleotide, 2′OMe-guanosine nucleotide, or mixtures thereof.

21. The siRNA molecule in accordance with claim 11, wherein said at least one modified nucleotide is in the sense strand of said siRNA molecule.

22. The siRNA molecule in accordance with claim 11, wherein less than about 30% of the nucleotides in said double-stranded region comprise modified nucleotides.

23. The siRNA molecule in accordance with claim 11, wherein less than about 20% of the nucleotides in said double-stranded region comprise modified nucleotides.

24. The siRNA molecule in accordance with claim 11, wherein said siRNA molecule is less immunostimulatory than a corresponding unmodified siRNA sequence.

25. The siRNA molecule in accordance with claim 1, wherein said siRNA molecule comprises a hairpin loop structure.

26. The siRNA molecule in accordance with claim 1, further comprising a carrier system.

27. The siRNA molecule in accordance with claim 26, wherein said carrier system is selected from the group consisting of a nucleic acid-lipid particle, a liposome, a micelle, a virosome, a nucleic acid complex, and a mixture thereof.

28. The siRNA molecule in accordance with claim 27, wherein said carrier system is a nucleic acid-lipid particle.

29. The siRNA molecule in accordance with claim 27, wherein said nucleic acid complex comprises said siRNA molecule complexed with a cationic lipid, a cationic polymer, a cyclodextrin, or a mixture thereof.

30. The siRNA molecule in accordance with claim 29, wherein said siRNA molecule is complexed with a cationic polymer, wherein said cationic polymer is polyethylenimine (PEI).

31. A pharmaceutical composition comprising an siRNA molecule in accordance with claim 1 and a pharmaceutically acceptable carrier.

32. A nucleic acid-lipid particle comprising:
an siRNA molecule in accordance with claim 1;
a cationic lipid; and
a non-cationic lipid.

33. The nucleic acid-lipid particle in accordance with claim 32, wherein the cationic lipid is a member selected from the group consisting of N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxypropylamine (DODMA), 1,2-DiLinoleyloxy-N,N-dimethylaminopropane (DLinDMA), 1,2-Dilinolenyloxy-N,N-dimethylaminopropane (DLendMA), and a mixture thereof.

34. The nucleic acid-lipid particle in accordance with claim 32, wherein the cationic lipid is DLinDMA.

35. The nucleic acid-lipid particle in accordance with claim 32, wherein the non-cationic lipid is an anionic lipid.

36. The nucleic acid-lipid particle in accordance with claim 32, wherein the non-cationic lipid is a neutral lipid.

37. The nucleic acid-lipid particle in accordance with claim 32, wherein the non-cationic lipid is a member selected from the group consisting of distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylcholine (DPPC), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), cholesterol, and a mixture thereof.

38. The nucleic acid-lipid particle in accordance with claim 32, wherein the non-cationic lipid is DSPC, DPPC, or DSPE.

39. The nucleic acid-lipid particle in accordance with claim 32, further comprising a conjugated lipid that inhibits aggregation of particles.

40. The nucleic acid-lipid particle in accordance with claim 39, wherein the conjugated lipid that inhibits aggregation of particles is a member selected from the group consisting of a polyethyleneglycol (PEG)-lipid conjugate, a polyamide (ATTA)-lipid conjugate, and a mixture thereof.

41. The nucleic acid-lipid particle in accordance with claim 40, wherein the PEG-lipid is a member selected from the group consisting of a PEG-diacylglycerol, a PEG dialkyloxypropyl, a PEG-phospholipid, a PEG-ceramide, and a mixture thereof.

42. The nucleic acid-lipid particle in accordance with claim 40, wherein the conjugated lipid that inhibits aggregation of particles comprises a polyethyleneglycol (PEG)-dialkyloxypropyl (PEG-DAA) conjugate.

43. The nucleic acid-lipid particle in accordance with claim 42, wherein the PEG-DAA conjugate is a member selected from the group consisting of a PEG-dilauryloxypropyl (C₁₂), a PEG-dimyristyloxypropyl (C₁₄), a PEG-dipalmityloxypropyl (C₁₆), and a PEG-distearyloxypropyl (C₁₈).

44. The nucleic acid-lipid particle in accordance with claim 42, wherein the PEG-DAA conjugate is a PEG-dimyristyloxypropyl (C₁₄).

45. The nucleic acid-lipid particle in accordance with claim 32, wherein the cationic lipid comprises from about 20 mol % to about 50 mol % of the total lipid present in the particle.

46. The nucleic acid-lipid particle in accordance with claim 32, wherein the cationic lipid comprises about 40 mol % of the total lipid present in the particle.

47. The nucleic acid-lipid particle in accordance with claim 32, wherein the non-cationic lipid comprises from about 5 mol % to about 90 mol % of the total lipid present in the particle.

48. The nucleic acid-lipid particle in accordance with claim 32, wherein the non-cationic lipid comprises about 20 mol % of the total lipid present in the particle.

49. The nucleic acid-lipid particle in accordance with claim 42, wherein the PEG-DAA conjugate comprises from 0 mol % to about 20 mol % of the total lipid present in the particle.

50. The nucleic acid-lipid particle in accordance with claim 42, wherein the PEG-DAA conjugate comprises about 2 mol % of the total lipid present in the particle.

51. The nucleic acid-lipid particle in accordance with claim 32, further comprising cholesterol.

52. The nucleic acid-lipid particle in accordance with claim 51, wherein the cholesterol comprises from about 10 mol % to about 60 mol % of the total lipid present in the particle.

53. The nucleic acid-lipid particle in accordance with claim 51, wherein the cholesterol comprises about 48 mol % of the total lipid present in the particle.

54. The nucleic acid-lipid particle in accordance with claim 32, wherein the nucleic acid in the nucleic acid-lipid particle is not substantially degraded after exposure of the particle to a nuclease at 37° C. for 20 minutes.

55. The nucleic acid-lipid particle in accordance with claim 32, wherein the nucleic acid in the nucleic acid-lipid particle is not substantially degraded after incubation of the particle in serum at 37° C. for 30 minutes.

56. The nucleic acid-lipid particle in accordance with claim 32, wherein the nucleic acid is fully encapsulated in the nucleic acid-lipid particle.

57. The nucleic acid-lipid particle in accordance with claim 32, wherein the particle has a nucleic acid:lipid mass ratio of from about 0.01 to about 0.2.

58. The nucleic acid-lipid particle in accordance with claim 32, wherein the particle has a nucleic acid:lipid mass ratio of from about 0.02 to about 0.1.

59. The nucleic acid-lipid particle in accordance with claim 32, wherein the particle has a nucleic acid:lipid mass ratio of about 0.08.

60. The nucleic acid-lipid particle in accordance with claim 32, wherein the particle has a median diameter of from about 50 nm to about 150 nm.

61. The nucleic acid-lipid particle in accordance with claim 32, wherein the particle has a median diameter of from about 70 nm to about 90 nm.

62. A pharmaceutical composition comprising a nucleic acid-lipid particle of claim 32 and a pharmaceutically acceptable carrier.

63. A method for introducing an siRNA that silences expression of an influenza virus gene into a cell, said method comprising:
contacting said cell with an siRNA molecule in accordance with claim 1.

64. The method in accordance with claim 63, wherein said siRNA molecule is in a carrier system.

65. The method in accordance with claim 64, wherein said carrier system is selected from the group consisting of a nucleic acid-lipid particle, a liposome, a micelle, a virosome, a nucleic acid complex, and a mixture thereof.

66. The method in accordance with claim 65, wherein said carrier system is a nucleic acid-lipid particle.

67. The method in accordance with claim 65, wherein said nucleic acid complex comprises said siRNA molecule complexed with a cationic lipid, a cationic polymer, a cyclodextrin, or a mixture thereof.

68. The method in accordance with claim 67, wherein said siRNA molecule is complexed with a cationic polymer, wherein said cationic polymer is polyethylenimine (PEI).

69. The method in accordance with claim 64, wherein said carrier system is a nucleic acid-lipid particle comprising:
said siRNA molecule;
a cationic lipid; and
a non-cationic lipid.

70. The in accordance with claim 69, wherein said nucleic acid-lipid particle further comprises a conjugated lipid that prevents aggregation of particles.

71. The method in accordance with claim 69, wherein the presence of said nucleic acid-lipid particle is detectable at least 1 hour after administration of said particle.

72. The method in accordance with claim 69, wherein more than 10% of a plurality of said particles are present in the plasma of a mammal about 1 hour after administration.

73. The method in accordance with claim 69, wherein an effect of the siRNA at a site distal to the site of administration is detectable for at least 72 hours after administration of said nucleic acid-lipid particle.

74. The method in accordance with claim 63, wherein said cell is in a mammal.

75. The method in accordance with claim 74, wherein said mammal is a human.

76. The method in accordance with claim 63, wherein said siRNA molecule comprises at least one of the sequences set forth in Tables 7-8.

77. A method for in vivo delivery of an siRNA that silences expression of an influenza virus gene, said method comprising:
administering to a mammalian subject an siRNA molecule in accordance with claim 1.

78. The method in accordance with claim 77, wherein said siRNA molecule is in a carrier system.

79. The method in accordance with claim 78, wherein said carrier system is selected from the group consisting of a nucleic acid-lipid particle, a liposome, a micelle, a virosome, a nucleic acid complex, and a mixture thereof.

80. The method in accordance with claim 79, wherein said carrier system is a nucleic acid-lipid particle.

81. The method in accordance with claim 79, wherein said nucleic acid complex comprises said siRNA molecule complexed with a cationic lipid, a cationic polymer, a cyclodextrin, or a mixture thereof.

82. The method in accordance with claim 81, wherein said siRNA molecule is complexed with a cationic polymer, wherein said cationic polymer is polyethylenimine (PEI).

83. The method in accordance with claim 78, wherein said carrier system is a nucleic acid-lipid particle comprising:
said siRNA molecule;
a cationic lipid; and
a non-cationic lipid.

84. The in accordance with claim 83, wherein said nucleic acid-lipid particle further comprises a conjugated lipid that prevents aggregation of particles.

85. The method in accordance with claim 83, wherein said mammal has been exposed to a second mammal infected with an influenza virus prior to administration of said nucleic acid-lipid particle.

86. The method in accordance with claim 83, wherein said mammal has been exposed to a fomite contaminated with an influenza virus prior to administration of said nucleic acid-lipid particle.

87. The method in accordance with claim 83, wherein administration of said nucleic acid-lipid particle reduces the amount of influenza hemagglutinin (HA) protein in said mammal by at least about 40% relative to the amount of influenza HA protein in the absence of said particle.

88. The method in accordance with claim 77, wherein said administration is selected from the group consisting of oral, intranasal, intravenous, intraperitoneal, intramuscular, intra-articular, intralesional, intratracheal, subcutaneous, and intradermal.

89. The method in accordance with claim 77, wherein said mammalian subject is a human.

90. The method in accordance with claim 77, wherein said siRNA molecule comprises at least one of the sequences set forth in Tables 7-8.

91. A method for modifying an anti-influenza siRNA having immunostimulatory properties, said method comprising:
(a) providing an unmodified siRNA sequence capable of silencing expression of an influenza virus gene selected from the group consisting of PA, PB1, PB2, NP, M1, M2, NS1, and NS2; and
(b) modifying said unmodified siRNA sequence by substituting at least one nucleotide in the sense or antisense strand with a modified nucleotide,
thereby generating a modified siRNA molecule that is less immunostimulatory than said unmodified siRNA sequence and is capable of silencing expression of said influenza virus gene.

92. The method in accordance with claim 91, wherein said modified nucleotide is selected from the group consisting of a 2′-O-methyl (2′OMe) nucleotide, 2′-deoxy-2′-fluoro (2′F) nucleotide, 2′-deoxy nucleotide, 2′-O-(2-methoxyethyl) (MOE) nucleotide, locked nucleic acid (LNA) nucleotide, and mixtures thereof.

93. The method in accordance with claim 91, wherein said modified nucleotide is a modified uridine nucleotide, modified guanosine nucleotide, or mixtures thereof.

94. The method in accordance with claim 91, wherein said unmodified siRNA sequence is modified by substituting all of the uridine nucleotides in the sense or antisense strand with modified uridine nucleotides.

95. The method in accordance with claim 94, further comprising at least one modified nucleotide selected from the group consisting of a modified guanosine nucleotide, modified adenosine nucleotide, modified cytosine nucleotide, and mixtures thereof.

96. The method in accordance with claim 91, wherein said modified nucleotide is a 2′OMe nucleotide.

97. The method in accordance with claim 91, wherein said modified nucleotide is selected from the group consisting of a 2′OMe-guanosine nucleotide, 2′OMe-uridine nucleotide, 2′OMe-adenosine nucleotide, and mixtures thereof.

98. The method in accordance with claim 91, wherein said modified nucleotide is not a 2′OMe-cytosine nucleotide.

99. The method in accordance with claim 91, wherein said modified nucleotide is a 2′OMe-uridine nucleotide, 2′OMe-guanosine nucleotide, or mixtures thereof.

100. The method in accordance with claim 91, further comprising:
(c) confirming that said modified siRNA molecule is less immunostimulatory by contacting said modified siRNA molecule with a mammalian responder cell under conditions suitable for said mammalian responder cell to produce a detectable immune response.

101. A method for identifying and modifying an anti-influenza siRNA having immunostimulatory properties, said method comprising:
(a) contacting an unmodified siRNA sequence capable of silencing expression of an influenza virus gene with a mammalian responder cell under conditions suitable for said mammalian responder cell to produce a detectable immune response, wherein said influenza virus gene is selected from the group consisting of PA, PB1, PB2, NP, M1, M2, NS1, and NS2;
(b) identifying said unmodified siRNA sequence as an immunostimulatory siRNA molecule by the presence of a detectable immune response in said mammalian responder cell; and
(c) modifying said immunostimulatory siRNA molecule by substituting at least one nucleotide with a modified nucleotide, thereby generating a modified siRNA molecule that is less immunostimulatory than said unmodified siRNA sequence.

102. The method in accordance with claim 101, wherein said modified nucleotide is selected from the group consisting of a 2′-O-methyl (2′OMe) nucleotide, 2′-deoxy-2′-fluoro (2′F) nucleotide, 2′-deoxy nucleotide, 2′-O-(2-methoxyethyl) (MOE) nucleotide, locked nucleic acid (LNA) nucleotide, and mixtures thereof.

103. The method in accordance with claim 101, wherein said modified nucleotide is a modified uridine nucleotide, modified guanosine nucleotide, or mixtures thereof.

104. The method in accordance with claim 101, wherein said immunostimulatory siRNA molecule is modified by substituting all of the uridine nucleotides in one strand with modified uridine nucleotides.

105. The method in accordance with claim 104, further comprising at least one modified nucleotide selected from the group consisting of a modified guanosine nucleotide, modified adenosine nucleotide, modified cytosine nucleotide, and mixtures thereof.

106. The method in accordance with claim 101, wherein said modified nucleotide is a 2′OMe nucleotide.

107. The method in accordance with claim 101, wherein said modified nucleotide is selected from the group consisting of a 2′OMe-guanosine nucleotide, 2′OMe-uridine nucleotide, 2′OMe-adenosine nucleotide, and mixtures thereof.

108. The method in accordance with claim 101, wherein said modified nucleotide is not a 2′OMe-cytosine nucleotide.

109. The method in accordance with claim 101, wherein said modified nucleotide is a 2′OMe-uridine nucleotide, 2′OMe-guanosine nucleotide, or mixtures thereof.

110. The method in accordance with claim 101, wherein said mammalian responder cell is a peripheral blood mononuclear cell.

111. The method in accordance with claim 101, wherein said detectable immune response comprises production of a cytokine or growth factor selected from the group consisting of TNF-α, IFN-α, IFN-β, IFN-γ, IL-6, IL-12, and combinations thereof.
